SEQUENCE LISTING (13 sequences)

SEQ ID NO: 1 - protein, 385 aa, Cannabis sativa
MNHLRAEGPASVLAIGTANPENILIQDEFPDYYFRVTKSEHMTQLKEKFRKICDKSMIRKRNCFLNEEHLKQNPRLVEHEMQTLDARQDMLVVEVPKLGKDACAKAIKEWGQPKSKITHLIFTSASTTDMPGADYHCAKLLGLSPSVKRVMMYQLGCYGGGTVLRIAKDIAENNKGARVLAVCCDIMACLFRGPSDSDLELLVGQAIFGDGAAAVIVGAEPDESVGERPIFELVSTGQTILPNSEGTIGGHIREAGLIFDLHKDVPMLISNNIEKCLIEAFTPIGISDWNSIFWITHPGGKAILDKVEEKLDLKKEKFVDSRHVLSEHGNMSSSTVLFVMDELRKRSLEEGKSTTGDGFEWGVLFGFGPGLTVERVVVRSVPIKY

SEQ ID NO: 2 - protein, 100 aa, Cannabis sativa
MAVKHLIVLKFKDEITEAQKEEFFKTYVNLVNIIPAMKDVYWGKDVTQKKEEGYTHIVEVTFESVETIQDYIIHPAHVGFGDVYRSFWEKLLIFDYTPRK

SEQ ID NO: 3 - protein, 101 aa, Cannabis sativa
MAVKHLIVLKFKDEITEAQKEEFFKTYVNLVNIIPAMKDVYWGKDVTQKNKEEGYTHIVEVTFESVETIQDYIIHPAHVGFGDVYRSFWEKLLIFDYTPRK

SEQ ID NO: 4 - protein, 720 aa, Cannabis sativa
MGKNYKSLDSVVASDFIALGITSEVAETLHGRLAEIVCNYGAATPQTWINIANHILSPDLPFSLHQMLFYGCYKDFGPAPPAWIPDPEKVKSTNLGALLEKRGKEFLGVKYKDPISSFSHFQEFSVRNPEVYWRTVLMDEMKISFSKDPECILRRDDINNPGGSEWLPGGYLNSAKNCLNVNSNKKLNDTMIVWRDEGNDDLPLNKLTLDQLRKRVWLVGYALEEMGLEKGCAIAIDMPMHVDAVVIYLAIVLAGYVVVSIADSFSAPEISTRLRLSKAKAIFTQDHIIRGKKRIPLYSRVVEAKSPMAIVIPCSGSNIGAELRDGDISWDYFLERAKEFKNCEFTAREQPVDAYTNILFSSGTTGEPKAIPWTQATPLKAAADGWSHLDIRKGDVIVWPTNLGWMMGPWLVYASLLNGASIALYNGSPLVSGFAKFVQDAKVTMLGVVPSIVRSWKSTNCVSGYDWSTIRCFSSSGEASNVDEYLWLMGRANYKPVIEMCGGTEIGGAFSAGSFLQAQSLSSFSSQCMGCTLYILDKNGYPMPKNKPGIGELALGPVMFGASKTLLNGNHHDVYFKGMPTLNGEVLRRHGDIFELTSNGYYHAHGRADDTMNIGGIKISSIEIERVCNEVDDRVFETTAIGVPPLGGGPEQLVIFFVLKDSNDTTIDLNQLRLSFNLGLQKKLNPLFKVTRVVPLSSLPRTATNKIMRRVLRQQFSHFE

SEQ ID NO: 5 - protein, 533 aa, Cannabis sativa
MEKSGYGRDGIYRSLRPPLHLPNNNNLSMVSFLFRNSSSYPQKPALIDSETNQILSFSHFKSTVIKVSHGFLNLGIKKNDWLIYAPNSIHFPVCFLGIIASGAIATTSNPLYTVSELSKQVKDSNPKLIITVPQLLEKVKGFNLPTILIGPDSEQESSSDKVMTFNDLVNLGGSSGSEFPIVDDFKQSDTAALLYSSGTTGMSKGWLTHKNFIASSLMVTMEQDLVGEMDNVFLCFLPMFHVFGLAIITYAQLQRGNTVISARFDLEKMLKDVEKYVTHLWWPPVILALSKNSMVKFNLSSIKYIGSGAAPLGKDLMEECSKWPYGIVAQGYGMTETCGIVSMEDIRGGKRNSGSAGMLASGVEAQIVSVDTLKPLPPNQLGEIWVKGPNMMQGYFNNPQATKLTIDKKGWVHTGDLGYFDEDGHLYWDRIKELIKYKGFQVAPAELEGLLVSHPEILDAWIPFPDAEAGEVPVAYWRSPNSSLTENDVKKFIAGQVASFKRLRKVTFINSVPKSASGKILRRELIQKVRSNM

SEQ ID NO: 6 - protein, 521 aa, Cannabis sativa
MEKSGYGRDGIYRSLRPPLHLPNNNNLSMVSFLFRNSSSYPQKPALIDSETNQILSFSHFKSTVIKVSHGFLNLGIKKNDWLIYAPNSIHFPVCFLGIIASGAIATTSNPLYTVSELSKQVKDSNPKLIITVPQLLEKVKGFNLPTILIGPDSEQESSSDKVMTFNDLVNLGGSSGSEFPIVDDFKQSDTAALLYSSGTTGMSKGWLTHKNFIASSLMVTMEQDLVGEMDNVFLCFLPMFHVFGLAIITYAQLQRGNTVISARFDLEKMLKDVEKYVTHLWWPPVILALSKNSMVKFNLSSIKYIGSGAAPLGKDLMEECSKWPYGIVAQGYGMTETCGIVSMEDIRGGKRNSGSAGMLASGVEAQIVSVDTLKPLPPNQLGEIWVKGPNMMQGYFNNPQATKLTIDKKGWVHTGDLGYFDEDGHLYWDRIKELIKYKGFQVAPAELEGLLVSHPEILDAWIPFPDAEAGEVPVAYWRSPNSSLTENDVKKFIAGQVASFKRLRKVTFINSVPKSASGKIL

SEQ ID NO: 7 - protein, 566 aa, Escherichia coli
MHPTGPHLGPDVLFRESNMKVTLTFNEQRRAAYRQQGLWGDASLADYWQQTARAMPDKIAVVDNHGASYTYSALDHAASCLANWMLAKGIESGDRIAFQLPGWCEFTVIYLACLKIGAVSVPLLPSWREAELVWVLNKCQAKMFFAPTLFKQTRPVDLILPLQNQLPQLQQIVGVDKLAPATSSLSLSQIIADNTSLTTAITTHGDELAAVLFTSGTEGLPKGVMLTHNNILASERAYCARLNLTWQDVFMMPAPLGHATGFLHGVTAPFLIGARSVLLDIFTPDACLALLEQQRCTCMLGATPFVYDLLNVLEKQPADLSALRFFLCGGTTIPKKVARECQQRGIKLLSVYGSTESSPHAVVNLDDPLSRFMHTDGYAAAGVEIKVVDDARKTLPPGCEGEEASRGPNVFMGYFDEPELTARALDEEGWYYSGDLCRMDEAGYIKITGRKKDIIVRGGENISSREVEDILLQHPKIHDACVVAMSDERLGERSCAYVVLKAPHHSLSLEEVVAFFSRKRVAKYKYPEHIVVIEKLPRTTSGKIQKFLLRKDIMRRLTQDVCEEIE

SEQ ID NO: 8 - DNA, 1701 bp, Arabidopsis thaliana
  source 1..1701: mol_type = genomic DNA
atggctgcgaccaaatggcgtgacatagatgatcttccaaaaattcccgctaattacacagctctgaccccactctggttccttgatcgcgctgccgttgtacaccccacgagaaaatcagttatccatggtagtagagaatacacatggagacaaacctacgatagatgtagaagattggcatctgctctagctgatagatccattggtccaggatctactgttgctattattgctccaaacattccagctatgtacgaagctcatttcggtgttccaatgtgtggtgctgttttgaactgtgttaacatccgcttgaacgctccgactgttgctttcttgttgtctcattctcaatcctctgttattatggttgatcaagaattcttcactttggctgaagattctttgagattgatggaagaaaaggctggttcctcattcaagagaccattgttgattgtaattggtgatcatacttgtgctccagaatcattgaacagagctttgtctaagggtgcaattgaatacgaagatttcttggctacaggtgatccaaactacccatggcaacctccagctgatgaatggcaatccattgctctcggttacacttctggtactaccgcaagcccaaagggtgtggttttgcatcatagaggtgcttacattatggctttgtctaacccattgatttggggtatgcaagatggtgctgtttacttgtggactttaccaatgttccattgtaacggttggtgtttcccatggtccttggcggttctgtctggtacttcgatttgtttgagacaagttactgctaaggaagtttactctatgattgctaagtacaaggttactcatttctgtgctgctccagttgttttgaacgctatcgttaacgctccaaaggaagatactattttgccattgccacatactgttcatgttatgactgctggtgctgcaccaccaccatctgtgttgttctctatgaaccaaaagggtttcagagttgctcatacttacggtctgtctgaaacttacggtccatctactgtttgtgcttggaagccagaatgggattcattgccaccagaaactcaagctaagttgaacgcaagacaaggtgttagatacactggtatggaacaattggatgttatcgatactcaaactggtaagcctgttccagctgatggtaagactgctggcgaaattgttttcagaggtaacatggttatgaagggttacttgaagaacccggaagctaacaaggaaacttttgcgggaggttggttccattctggtgatattgctgtcaagcatccagataactacattgaaattaaggatagatctaaggatgtgattatttctggtggtgaaaacatttcatctgttgaagttgaaaacgtcgtctaccatcatccagctgttttggaggcttctgttgttgctagaccagatgaaagatggcaagaatctccatgtgctttcgttactttgaagtccgattacgaaaagcatgatcaaaacaagttagctcaagatattatgaagttctgtagagaaaagttgccagcttactgggttccaaagtctgttgtgttcggcccattgcctaagactgctactggtaagattcagaagcatatcttgagaactaaggctaaggaaatgggtccagttccaagataa

SEQ ID NO: 9 - protein, 566 aa, Arabidopsis thaliana
MAATKWRDIDDLPKIPANYTALTPLWFLDRAAVVHPTRKSVIHGSREYTWRQTYDRCRRLASALADRSIGPGSTVAIIAPNIPAMYEAHFGVPMCGAVLNCVNIRLNAPTVAFLLSHSQSSVIMVDQEFFTLAEDSLRLMEEKAGSSFKRPLLIVIGDHTCAPESLNRALSKGAIEYEDFLATGDPNYPWQPPADEWQSIALGYTSGTTASPKGVVLHHRGAYIMALSNPLIWGMQDGAVYLWTLPMFHCNGWCFPWSLAVLSGTSICLRQVTAKEVYSMIAKYKVTHFCAAPVVLNAIVNAPKEDTILPLPHTVHVMTAGAAPPPSVLFSMNQKGFRVAHTYGLSETYGPSTVCAWKPEWDSLPPETQAKLNARQGVRYTGMEQLDVIDTQTGKPVPADGKTAGEIVFRGNMVMKGYLKNPEANKETFAGGWFHSGDIAVKHPDNYIEIKDRSKDVIISGGENISSVEVENVVYHHPAVLEASVVARPDERWQESPCAFVTLKSDYEKHDQNKLAQDIMKFCREKLPAYWVPKSVVFGPLPKTATGKIQKHILRTKAKEMGPVPR

SEQ ID NO: 10 - DNA, 1692 bp, Arabidopsis thaliana
  source 1..1692: mol_type = genomic DNA
atggctgccacacacttacatattccccccaatcctaagactcagacgtcgcaccaaaacccacccttttggtttagctccaaaaccggtatctatacatctaagtttccatcgctacacttgccagtggatccaaacttggatgctgtttctgctttgttctcccataagcatcatggtgatactgctttgattgattctttgactggtttctctatttcccatactgaattgcaaattatggttcaatctatggctgctggtatttaccatgttttgggtgtgagacaaggtgatgttgtctctttggttttgccaaactctgtttacttcccaatgattttcttgtctttgatttctctcggtgcgatagttactactatgaacccatcctcttctttgggtgaaattaagaagcaagtttctgaatgttctgttggtttagctttcacttctactgaaaacgttgaaaagttatcttctttgggtgtttctgttatttctgtttctgaatcttacgatttcgattctattagaattgaaaacccaaagttctactctattatgaaggaatccttcggtttcgttccaaagccattgattaagcaagatgacgttgctgccattatgtactctagcggtaccactggtgcttctaagggtgttcttcttacccatagaaacctaattgcttctatggaattgttcgttagattcgaagcttctcaatacgaatacccaggttcttctaacgtttacttggctgctttaccattgtgtcatatttacggcttgtctttgttcgttatgggtttgttgtctttgggtagcacgattgttgtaatgaagagattcgatgcttcagatgttgtaaacgttatagaaagattcaagattacacatttcccagtcgttccaccaatgttgatggctctcactaagaaggctaagggtgtttgtggtgaagttttcaagtctttgaagcaagtttcttccggtgctgctccattgtctagaaagttcattgaagatttcttacaaactttgccacatgttgatttgattcaaggttacggtatgactgaatcgactgctgttggtactagaggtttcaactctgaaaagttgtctagatactcttctgttggtttgcttgctccaaacatgcaagctaaggttgtcgattggtcttctggttcttttttgccaccaggtaacagaggcgaattgtggattcaaggtccaggtgttatgaagggttacttgaacaacccaaaggctacccaaatgtctattgttgaagattcctggttgcgtactggtgatattgcttacttcgatgaagatggttacttgttcattgttgataggattaaggaaattattaagtacaagggtttccaaatagctccagcagatttggaagctgttttggtttctcatccattgatcattgatgctgctgttactgctgcgcctaacgaagaatgtggtgaaattccagttgctttcgttgttagacgacaagaaaccactttatcagaagaagatgttattagctacgttgcttctcaagttgctccatacagaaaggttagaaaggttgttatggtcaactctattccaaagtctccaactggtaagattttgagaaaggaattgaagaggattttgactaactctgtttcttaa

SEQ ID NO: 11 - protein, 563 aa, Arabidopsis thaliana
MAATHLHIPPNPKTQTSHQNPPFWFSSKTGIYTSKFPSLHLPVDPNLDAVSALFSHKHHGDTALIDSLTGFSISHTELQIMVQSMAAGIYHVLGVRQGDVVSLVLPNSVYFPMIFLSLISLGAIVTTMNPSSSLGEIKKQVSECSVGLAFTSTENVEKLSSLGVSVISVSESYDFDSIRIENPKFYSIMKESFGFVPKPLIKQDDVAAIMYSSGTTGASKGVLLTHRNLIASMELFVRFEASQYEYPGSSNVYLAALPLCHIYGLSLFVMGLLSLGSTIVVMKRFDASDVVNVIERFKITHFPVVPPMLMALTKKAKGVCGEVFKSLKQVSSGAAPLSRKFIEDFLQTLPHVDLIQGYGMTESTAVGTRGFNSEKLSRYSSVGLLAPNMQAKVVDWSSGSFLPPGNRGELWIQGPGVMKGYLNNPKATQMSIVEDSWLRTGDIAYFDEDGYLFIVDRIKEIIKYKGFQIAPADLEAVLVSHPLIIDAAVTAAPNEECGEIPVAFVVRRQETTLSEEDVISYVASQVAPYRKVRKVVMVNSIPKSPTGKILRKELKRILTNSVS

SEQ ID NO: 12 - DNA, 21 bp, synthetic construct
  source 1..21: mol_type = other DNA
ggtggtggtccagaacaattg

SEQ ID NO: 13 - DNA, 25 bp, synthetic construct
  source 1..25: mol_type = other DNA
gcaaatcacgtgatatagatccacg

(19) United States
(12) Patent Application Publication
(10) Pub. No.: US 2025/0122539 A1
(43) Pub. Date: Apr. 17, 2025

(54) LARGE SCALE PRODUCTION OF DIVARIN, DIVARINIC ACID AND OTHER ALKYL RESORCINOLS BY FERMENTATION

(71) Applicant: LYGOS, INC., Hayward, CA (US)

(72) Inventors: Andrew Conley, Hayward, CA (US); Azedeh Alikhani, Hayward, CA (US); Rebecca Lennen, Hayward, CA (US)

(21) Appl. No.: 18/686,558

(22) PCT Filed: Aug. 25, 2022
(86) PCT No.: PCT/US2022/041498

§ 371 Date: Feb. 26, 2024

Related U.S. Application Data
(60) Provisional application No. 63/237,321, filed on Aug. 26, 2021.
(60) Provisional application No. 63/244,660, filed on Sep. 15, 2021.

(51) Int. Cl. C12P 7/22; C12P 7/42

(52) U.S. Cl. CPC C12P 7/22; C12P 7/42

(57) ABSTRACT

Provided herein are processes, such as commercially viable processes, of producing alkyl resorcinols, such as divarin and divarinic acid, and analogs of each thereof. Certain of these processes utilize a recombinant, heterologous host microorganism. Certain of the heterologous microorganisms include a Cannabis sativa olivetol synthase (which is a tetraketide synthase, csOLS). Certain of the heterologous microorganisms include a Cannabis sativa olivetolic acid cyclase (csOAC). Certain of the heterologous microorganisms include an acyl activating enzyme (cSAAE), such as, without limitation, csAAE1, csAAE7, AtAAE7, or At4CLL6. In certain of these processes, glucose is fermented. In certain of these processes, the fermentation further comprises a carboxylic acid, RCO₂H where R is defined as herein, or a salt thereof. Certain of these processes provide divarin and divarinic acid in a combined amount of at least 3 g/liter.

### PRIORITY CLAIM

This application claims priority to US provisional application nos. U.S. 63/237,321 and U.S. 63/244,660, each of which is incorporated herein in its entirety by reference.

### STATEMENT ABOUT FEDERAL FUNDING

Not applicable.

### FIELD

Provided herein are processes, preferably scalable, commercially relevant processes, of producing divarin and divarinic acid, or an analog thereof, or a salt of each thereof by fermentation employing a recombinant heterologous host microorganism.

### BACKGROUND

Olivetol and olivetolic acid are key gateway molecules for preparing cannabinoids. And yet, there is very little if any report of a scalable, commercially viable, production of olivetol and olivetolic acid by fermentation.

Further, divarin, which is 1,3-dihydroxy-5-propylbenzene and divarinic acid, which is 2,4-dihydroxy-6-propylbenzoic acid, are key gateway molecules for preparing certain minor cannabinoids. Minor cannabinoids are naturally obtained in quantities smaller than major cannabinoids such as tetrahydrocannabinol (THC). And yet, there is very little if any report of producing divarin and divarinic acid by fermentation.

### SUMMARY

In one aspect provided herein are processes of producing a compound of formula (IA) and/or (IB)

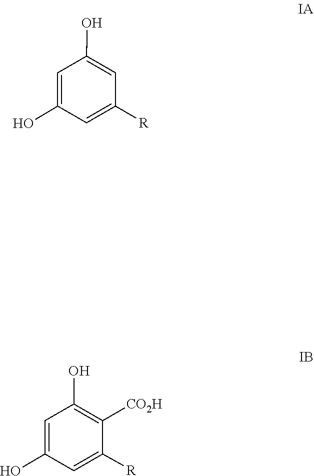

or a salt thereof wherein R is optionally substituted C₁-C₈ alkyl, optionally substituted C₂-C₆ alkenyl, or optionally substituted C₂-C₈ alkynyl. In certain embodiments, other R groups such as optionally substituted cycloalkyl, preferably optionally substituted C₃-C₈ cycloalkyl; optionally substituted heterocyclyl; optionally substituted aryl, preferably optionally substituted phenyl; and optionally substituted heteroaryl are contemplated as employed according to the present invention. In one embodiment, the compound produced is of formula IA. In another embodiment, the compound produced is of formula IB.

Certain of these processes utilize a recombinant, heterologous, host cells or host microorganism. Certain of the host microorganisms comprise a recombinant olivetol synthase (OLS or OS), which is a tetraketide synthase (TKS). Certain of the host microorganisms comprise a recombinant Cannabis sativa olivetol synthase (which is a tetraketide synthase, csOLS). Certain of the host microorganisms comprise a recombinant olivetolic acid cyclase (OAC). Certain of the heterologous microorganisms comprise a recombinant Cannabis sativa olivetolic acid cyclase (csOAC). Certain of the host microorganisms comprise an acyl activating enzyme (AAE). Certain of the heterologous microorganisms comprise a recombinant Cannabis sativa acyl activating enzyme (csAAE), such as, without limitation, csAAE1.

Certain of the heterologous microorganisms comprise a recombinant acyl activating enzyme (AAE), having at least 50%, or at least 75%, or at least 90%, or at least 95% sequence identity with a sequence selected from SEQ ID NO:8, SEQ ID NO:9, SEQ ID NO:10, or SEQ ID NO: 11. In one embodiment, the sequence has at least 50%, or at least 75%, or at least 90%, or at least 95% sequence identity with a sequence selected from SEQ ID NO:8. In one embodiment, the sequence has at least 50%, or at least 75%, or at least 90%, or at least 95% sequence identity with a sequence selected from SEQ ID NO:9. In one embodiment, the sequence has at least 50%, or at least 75%, or at least 90%, or at least 95% sequence identity with a sequence selected from SEQ ID NO:10. In one embodiment, the sequence has at least 50%, or at least 75%, or at least 90%, or at least 95% sequence identity with a sequence selected from SEQ ID NO:11. In one embodiment, the sequence has at least 50% sequence identity. In one embodiment, the sequence has at least 75% sequence identity. In one embodiment, the sequence has at least 90% sequence identity. In one embodiment, the sequence has at least 95% sequence identity. In one embodiment, the recombinant acyl activating enzyme (AAE) has SEQ ID NO:8. In one embodiment, the recombinant acyl activating enzyme (AAE) has SEQ ID NO:9. In one embodiment, the recombinant acyl activating enzyme (AAE) has SEQ ID NO:10. In one embodiment, the recombinant acyl activating enzyme (AAE) has SEQ ID NO:11.

In another embodiment, the recombinant host microorganism comprises 4-20, or 6-16 copies of csOLS. In another embodiment, the recombinant host microorganism comprises 4-20, or 6-16 copies of csOAC. In another embodiment, the recombinant host microorganism comprises 4-20, or 6-16 copies of csAAE1.

In certain of these processes, glucose is fermented. In certain of these processes, galactose is fermented. The process further comprises a carboxylic acid of formula R—CO₂H or a salt thereof. In certain of the processes, the microorganism is a yeast. In certain of the processes, the microorganism is Saccharomyces cerevisiae. In certain of the processes, the microorganism is a bacteria. In certain of the processes, the microorganism is Escherichia coli.

In one embodiment, a strain JK9-3d strain of Saccharomyces cerevisiae was employed. In comparison to the S. cerevisiae strain CEN.PK2-1C, integrations into S. cerevisiae JK9-3d were surprisingly more efficient and resulted in higher titers of olivetolic acid and divarinic acid.

In some embodiments, the process further comprises contacting: an aqueous phase comprising glucose and RCO₂H or a salt thereof and an organic phase immiscible with the aqueous phase.

In one embodiment, R is C₁-C₈ alkyl. In another embodiment, R is C₁-C₄ alkyl. In another embodiment, R is C₆-C₈ alkyl. In another embodiment, R is substituted C₁-C₈ alkyl.

In another embodiment, R is C₂-C₈ alkenyl. In another embodiment, R is substituted C₂-C₈ alkenyl.

In another embodiment, R is C₂-C₈ alkynyl. In another embodiment, R is substituted C₂-C₈ alkynyl.

In another embodiment, the compounds of formula IA and IB are provided in a combined amount of at least about 2 g/liter over about 4 to about 7 days. In another embodiment, the compounds of formula IA and IB are provided in a combined amount of at least about 3 g/liter over about 4 to about 7 days. In another embodiment, the compounds of formula IA and IB are provided in a combined amount of at least about 4 g/liter over about 4 to about 7 days. In another embodiment, the compounds of formula IA and IB are provided in a combined amount of at least about 5 g/liter over about 4 to about 7 days. In another embodiment, the compounds of formula IA and IB are provided in a combined amount of at least about 10 g/liter over about 4 to about 7 days.

This invention arises in part from the surprising discovery that recombinant host microorganisms produce commercially relevant amounts of olivetol and olivetolic acid by fermentation. In some aspects, provided herein are processes of producing olivetol, olivetolic acid, or a salt thereof. Certain of these processes are commercially viable for producing olivetol, olivetolic acid or a salt thereof, which are key gateway compounds for preparing a variety of cannabinoids. Certain of these processes utilize a recombinant, heterologous, host microorganism. Certain of the host microorganisms include a recombinant Cannabis sativa olivetol synthase (which is a tetraketide synthase, csOLS). Certain of the heterologous microorganisms include a recombinant Cannabis sativa olivetolic acid cyclase (csOAC). Certain of the heterologous microorganisms include a recombinant Cannabis sativa acyl activating enzyme (csAAE), such as, without limitation, csAAE1. In another embodiment, the recombinant host microorganism comprises 4-20, or 6-16 copies of csOLS. In another embodiment, the recombinant host microorganism comprises 4-20, or 6-16 copies of csOAC. In another embodiment, the recombinant host microorganism comprises 4-20, or 6-16 copies of csAAE1. In certain of these processes, glucose is fermented. In certain of these processes, galactose is fermented. In certain of these processes, the fermentation further comprises hexanoic acid or a salt thereof. Certain of these processes provide olivetol and olivetolic acid in a combined amount of at least 3 g/liter. In certain of the processes, the microorganism is Saccharomyces cerevisiae.

This invention arises in another part from the surprising discovery that recombinant host microorganisms produce divarin and divarinic acid by fermentation. In some aspects, provided herein are processes of producing divarin and/or divarinic acid or a salt thereof. Certain of these processes are commercially viable for producing divarin and divarinic acid, which are key gateway compounds for preparing a variety of minor cannabinoids. Certain of these processes utilize a recombinant, heterologous, host microorganism. Certain of the host microorganisms include a recombinant Cannabis sativa olivetol synthase (which is a tetraketide synthase, csOLS). Certain of the heterologous microorganisms include a recombinant Cannabis sativa olivetolic acid cyclase (csOAC). Certain of the heterologous microorganisms include a recombinant acyl activating enzyme (AAE), such as, without limitation, csAAE1. In another embodiment, the recombinant host microorganism comprises 4-20, or 6-16 copies of csOLS. In another embodiment, the recombinant host microorganism comprises 4-20, or 6-16 copies of csOAC. In another embodiment, the recombinant host microorganism comprises 4-20, or 6-16 copies of an AAE. In certain of these processes, glucose is fermented. In certain of these processes, the fermentation further comprises butyric acid or a salt thereof. Certain of these processes provide divarin and/or divarinic acid or a salt thereof in a combined amount of at least about 0.25-about 8 g/liter, about 1-about 7 g/liter, about 0.25-about 2 g/liter, about 0.25-about 2 g/liter, about 0.5-about 1 g/liter, or about 2-about 4 g/liter. Certain of these processes provide divarin and/or divarinic acid or a salt thereof in a combined amount of at least about 2-about 5, preferably about 3-about 4 g/liter. In one embodiment, the combined amount of divarin and/or divarinic acid is provided over 2-7 or 4-7 days, such as 2, 3, 4, 5, 6, or 7 days. In certain of the processes, the microorganism is Saccharomyces cerevisiae.

In one embodiment, the fermentation is performed as a batch/fed batch fermentation with a fixed batch duration. In one embodiment, the fermentation is performed as a “semi-continuous” fermentation operating mode. In one embodiment, the fermentation is performed as a continuous fermentation operating mode. The continuous mode may be a fill-and-draw, or a true continuous operation.

An illustrative and non-limiting process of isolating olivetol or another compound of formula IA or IB is schematically illustrated in FIG. 1.

In one embodiment, a mixture of compounds of formula IA and IB provided by fermentation is extracted from a fermentation media by alkaline extraction. In some embodiments, the alkaline extraction is an aqueous alkaline extraction. In some embodiments, the alkaline extraction is performed at a pH of about 12-about 14. In some embodiments, the alkaline extraction is performed at a pH of about 13. In some embodiments, the alkaline extraction is performed under milder alkaline conditions. In some embodiments, the alkaline extraction is performed at a pH of about 7-about 12. In some embodiments, the alkaline extraction is performed at a pH of about 7-about 10. Without being bound by theory, under the milder alkaline extraction, a compound of formula IB is preferentially extracted. The compound of formula IA can thereafter be extracted under stronger alkaline conditions, e.g., as described herein.

In some embodiments, the extracted mixture of compounds of formula IA and IB are decarboxylated to provide a compound of formula IA. In some embodiments, the decarboxylation is performed by heating. In some embodiments, the heating is performed at about 100° C.-about 140° C., or preferably at about 110° C.-about 130° C. In some embodiments, the heating is performed at about 120° C. Post decarboxylation, the compound of formula IA provided, comprises by weight about 2% or less, or preferably about 1% or less of a compound of formula IB or a salt thereof. In some embodiments, the extracted mixture of compounds of formula IA and IB are acidified before decarboxylation. In some embodiments, the decarboxylation is performed at a pH of about 5-about 8. In some embodiments, the decarboxylation is performed at a pH of about 6.5.

In one embodiment, the compound of formula IA provided by decarboxylation is extracted into an organic solvent (e.g., a water immiscible organic solvent) to provide a solution of the compound of formula IA in the organic solvent. In some embodiments, the organic solvent is a solvent capable of dissolving a compound of formula IA; formula IA comprises an aromatic ring and polar hydroxy groups. In one embodiment, the organic solvent comprises an aromatic hydrocarbon solvent. In one embodiment, the organic solvent comprises toluene. In one embodiment, the organic solvent is toluene. In some embodiments, the organic solvent comprises aliphatic or alicyclic hydrocarbon solvents.

In some embodiments, the compound of formula IA, present as a solution in the organic solvent, is reacted with a terpene alcohol, a terpenal (i.e., a terpene aldehyde), and the likes. In some embodiments, the solution of the compound of formula IA in the organic solvent is employed for reacting the compound of formula IA with a terpene alcohol. In some embodiments, the solution of the compound of formula IA in the organic solvent is employed for reacting the compound of formula IA with a terpenal. In one embodiment, the terpene alcohol is geraniol. In one embodiment, the terpene alcohol is farnesol. In one embodiment, the terpene alcohol is menthadienol (trans 2,8-menthadienol or PMD). In one embodiment, the terpene alcohol is

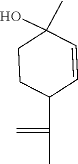

or a diastereomer thereof, or an ester of each thereof. In one embodiment, the the hydroxy form (unesterified) is employed. In one embodiment, the terpene alcohol is:

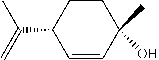

### (1R,4R)-4-Isopropenyl-1-methyl-2-cyclohexen-1-ol

In one embodiment, the terpenal is citral. In some embodiments, the reaction with a terpenal further comprises a primary amine. In one embodiment, the primary amine is tertiary butyl amine.

In some embodiments, the reaction of a compound of formula IA with a terpene alcohol, a terpenal, or the likes provides a cannabinoid. In one embodiment, the cannabinoid is cannabigerol (CBG). In another embodiment, the cannabinoid is cannabichromene (CBC). In another embodiment, the cannabinoid is cannabidiol (CBD). In another embodiment, the cannabinoid is tetrahydrocannabinol (THC). In another embodiment, the cannabinoid is cannabinol (CBN). In another embodiment, the cannabinoid is the varin analog (CBGV, CBCV, CBDV, THCV, CBNV) of CBG, CBC, CBD, THC, CBN. A varin analog is a compound where the n-pentyl chain of a cannabinoid, e.g., and without limitation, CBG, CBC, CBD, or THC is replaced by an n-propyl chain. The cannabinoids obtained are purified by a variety of purification methods. In one embodiment, the purification method comprises chromatography. In one embodiment the purification method comprises distillation. In one embodiment, the chromatography comprises a reverse phase chromatography.

In one embodiment, R is n-pentyl. In another embodiment, R is n-propyl. In another embodiment, R is n-heptyl.

A non-limiting example of reacting (prenylating) olivetol with the terpene alcohol, geraniol, is schematically illustrated in FIG. 2.

A non-limiting example of reacting (prenylating) olivetol with the terpenal, citral, is schematically illustrated in FIG. 3.

The initial engineering of Saccharomyces cerevisiae was done by introducing a gene fragment containing csOLS, csOAC, and csAAE1 under the control of galactose regulatable elements called promoters. The csOLS and csOAC were physically linked to each other on the gene with a genetic element called T2A in all examples.

To select for Saccharomyces cerevisiae cells that efficiently incorporated the foreign DNA, but removed Saccharomyces cerevisiae that had no foreign genes, a standard protocol method was utilized that allows growth on nutrient preferred media. One way to construct gene fragments that are functional in an organism is to generate individual gene fragments by a polymerase chain reaction (PCR). This creates individual gene fragments from simple smaller DNA sequences and a well defined DNA fragment called a ‘template’ that contains pieces of your final gene fragment. The smaller pieces of DNA are called ‘primers’. These primers flank the DNA you want to generate from various templates to generate the final product you desire by PCR. These final products are called ‘amplicons’. One process to ‘stitch’ together various amplicons is called Gibson Assembly. Many gene fragments disclosed in this method were first generated by Gibson Assembly of several amplicons generated by PCR. These assembled gene fragments were then allowed to be uptaken iteratively into a wild type Saccharomyces cerevisiae cell called JK9-3d. In some embodiments, CEN.PK is useful as a wild type Saccharomyces cerevisiae.

The process by which Saccharomyces cerevisiae uptakes foreign DNA and stably utilize the foreign DNA is called recombination. The final Saccharomyces cerevisiae strains that took up the foreign DNA and utilized the DNA are called recombinants. Saccharomyces cerevisiae that did not undergo recombination are the wild type. The process of selecting recombinants in a preferred media is termed prototrophy rescue. To separate recombinants from wild type prototrophy rescue was utilized.

Examples herein below provide a method to create recombinants that produce various levels of O/OA by varying how many of those gene fragments are uptaken by JK9-3d. In one example, recombinants that produce O/OA under the control of galactose are disclosed. Another example discloses, how the number of exogenous fragments taken up from Saccharomyces cerevisiae correlates with O/OA concentrations in the media. The number of genetic fragments recombinants contain can be determined by sequencing the DNA of the recombinant and by using the PCR method to quantify the number of amplicons generated. Amplicons are quantified by quantitative real time polymerase chain reaction (qPCR). qPCR and direct sequencing, and how those quantitative values of the genetic elements relate to the quantitative levels of O and OA are exemplified and provided herein.

When jointly expressing two iterative fungal polyketide synthases, consisting of a highly reducing PKS (HRPKS) and a nonreducing PKS (NRPKS), macrolactones known as resorcylic acid lactones can be formed that can be subsequently released by an un-fused (pseudoacyl carrier protein)-thioesterase (TE) to produce olivetolic acid and the related octanoyl-primed derivative sphaerophorolcarboxylic acid, along with other 6-alkyl-substituted 2,4-dihydroxybenzoic acids. In these instances, the TEs are typically free-standing enzymes and catalyze a hydrolysis reaction instead of esterification. Mixing and matching of different HRPKSs and NRPKSs produce different OA analogs, e.g., and without limitation of formula IB, including those that can lead to rare or unnatural cannabinoids.

For example, and without limitation, the ova cluster from Metarhizium anisopliae encodes a typical HRPKS (Ma_OvaA) and an NRPKS (Ma_OvaB) along with a (pseudoacyl carrier protein)-thioesterase (MaOvaC). Homologous clusters like this have been observed in various fungal species such as Metarhizium rileyi (gene cluster Mr_OvaABC), Talaromyces islandicus (gene cluster Ti_OvaABC), Tolypocladium inflatum (gene cluster To_OvaABC), Metarhizium guizhouense (gene cluster Mg_OvaABC), Metarhizium album (gene cluster Ma_OvaABC), Ophiocordyceps australis (gene cluster Oa_OvaABC), and Cladobotryum varium (gene cluster Cv_OvaABC). When this trio of HRPKS, NRPKS and (pseudoacyl carrier protein)-thioesterase enzymes are co-expressed in the fungus Aspergillus nidulans, olivetolic acid and related compounds, e.g., and without limitation of formula IB, are produced with either saturated or unsaturated alkyl chains. In one embodiment, the host microorganism is Aspergillus nidulans. In some embodiments, a carboxylic acid of formula RCO₂H, such as hexanoic acid, butyric acid, and the likes, is not externally added to the fermentation comprising Aspergillus nidulans.

### DESCRIPTION OF THE FIGURES

FIG. 1 schematically illustrates recovery of olivetol and other compounds of formula IA in accordance with the present invention.

FIG. 2 schematically illustrates the semisynthesis of cannabinoids (CBG) by prenylation of fermented olivetol.

FIG. 3 schematically illustrates the semisynthesis of cannabinoids (CBC) by prenylation of fermented olivetol.

FIGS. 4A-B, 5A-D, and 6A-D graphically illustrate production of certain alkyl resorcinol and alkyl resorcinol carboxylic acids as provided herein.

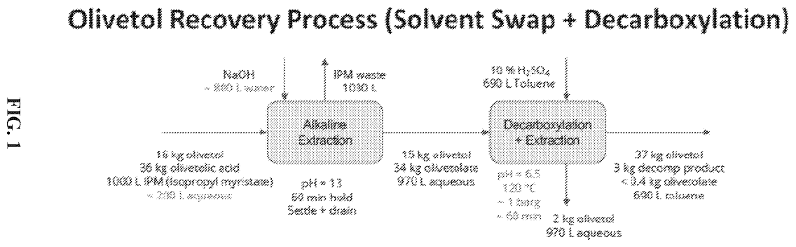

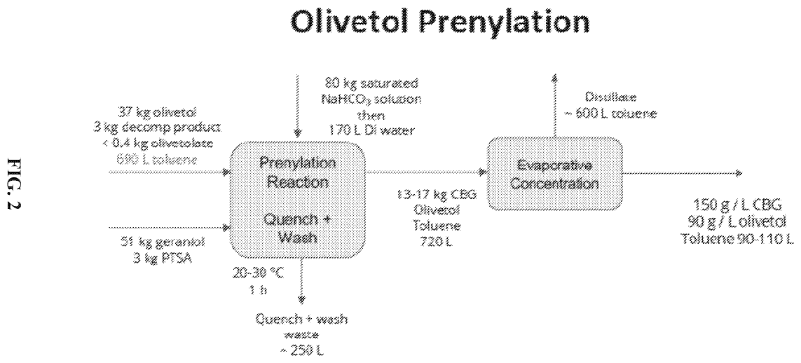

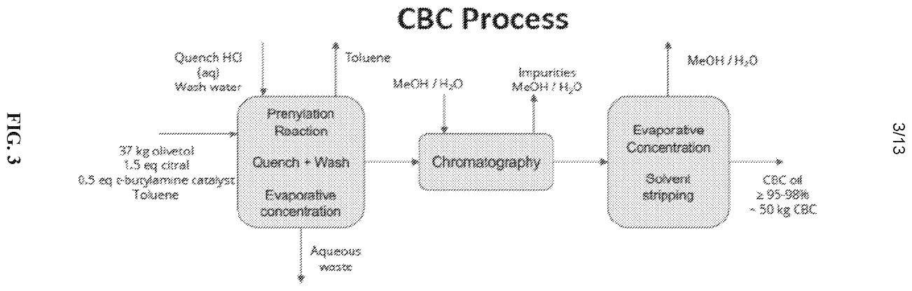

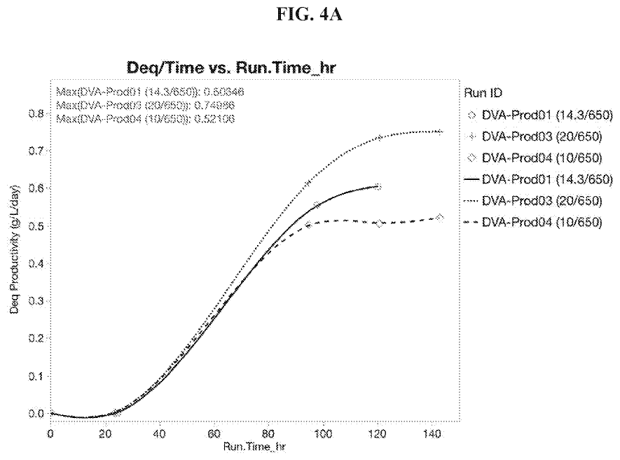

### DETAILED DESCRIPTION

While the present invention is described herein with reference to aspects and specific embodiments thereof, those skilled in the art will recognize that various changes may be made and equivalents may be substituted without departing from the invention. The present invention is not limited to particular nucleic acids, expression vectors, enzymes, host microorganisms, or processes, as such may vary. The terminology used herein is for purposes of describing particular aspects and embodiments only, and is not to be construed as limiting. In addition, many modifications may be made to adapt a particular situation, material, composition of matter, process, process step or steps, in accordance with the invention. All such modifications are within the scope of the claims appended hereto. Headers are used solely for readers' convenience, and disclosure found under any header is understood in the context of and applicable to the entire disclosure.

### Definitions

In this specification and in the claims that follow, reference will be made to a number of terms that shall be defined to have the following meanings.

As used in the specification and the appended claims, the singular forms “a”, “an”, and “the” include plural referents unless the context clearly dictates otherwise. Thus, for example, reference to an “expression vector” includes a single expression vector as well as a plurality of expression vectors, either the same (e.g., the same operon) or different; reference to “cell” includes a single cell as well as a plurality of cells; and the like.

As used herein, the term “comprising” is intended to mean that the compounds, compositions and processes include the recited elements, but not exclude others. “Consisting essentially of” when used to define compounds, compositions and processes, shall mean excluding other elements of any essential significance to the combination. Thus, a composition consisting essentially of the elements as defined herein would not exclude trace contaminants, e.g., from the isolation and purification method. “Consisting of” shall mean excluding more than trace elements of other ingredients. Embodiments defined by each of these transition terms are within the scope of this technology.

All numerical designations, e.g., pH, temperature, time, concentration, and molecular weight, including ranges, are approximations which are varied (+) or (−) by increments of 1, 5, or 10%, e.g., by using the prefix, “about.” It is to be understood, although not always explicitly stated that all numerical designations are preceded by the term “about.” It also is to be understood, although not always explicitly stated, that the reagents described herein are merely exemplary and that equivalents of such are known in the art.

“Alkyl” refers to monovalent saturated aliphatic hydrocarbyl groups having from 1 to 10 carbon atoms and preferably 1 to 6 carbon atoms. Higher carbon atom containing alkyl groups are also contemplated in certain embodiments, as the context will indicate. This term includes, by way of example, linear and branched hydrocarbyl groups such as methyl (CH₃—), ethyl (CH₃CH₂), -n-propyl-(CH₃CH₂CH₂—), isopropyl ((CH₃)₂CH), -n-butyl-(CH₃CH₂CH₂CH₂—), isobutyl ((CH₃)₂CHCH₂—), sec-butyl ((CH₃)(CH₃CH₂)CH), -t-butyl-((CH₃)₃C), -n-pentyl-(CH₃CH₂CH₂CH₂CH₂—), and neopentyl ((CH₃)₃CCH₂—).

“Alkenyl” refers to monovalent straight or branched hydrocarbyl groups having from 2 to 10 carbon atoms and preferably 2 to 6 carbon atoms or preferably 2 to 4 carbon atoms and having at least 1 and preferably from 1 to 2 sites of vinyl (>C═C<) unsaturation. Higher carbon atom containing alkenyl groups are also contemplated in certain embodiments, as the context will indicate. Such groups are exemplified, for example, by vinyl, allyl, and but-3-en-1yl. Included within this term are the cis and trans isomers or mixtures of these isomers.

“Alkynyl” refers to straight or branched monovalent hydrocarbyl groups having from 2 to 10 carbon atoms and preferably 2 to 6 carbon atoms or preferably 2 to 3 carbon atoms and having at least 1 and preferably from 1 to 2 sites of acetylenic (—C≡C—) unsaturation. Higher carbon atom containing alkynyl groups are also contemplated in certain embodiments, as the context will indicate. Examples of such alkynyl groups include acetylenyl (—C≡CH), and propargyl (—CH₂C≡CH).

“Substituted alkyl” refers to an alkyl group having from 1 to 5, preferably 1 to 3, or more preferably 1 to 2 substituents selected from the group consisting of alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, substituted sulfonyloxy, thioacyl, thiol, alkylthio, and substituted alkylthio, wherein said substituents are as defined herein.

“Heteroalkyl” refers to an alkyl group one or more carbons is replaced with —O—, —S—, SO₂, a phosphorous (P) containing moiety, or —NR^{Q}— moieties where R^{Q} is H or C₁-C₆ alkyl. Substituted heteroalkyl refers to a heteroalkyl group having from 1 to 5, preferably 1 to 3, or more preferably 1 to 2 substituents selected from the group consisting of alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester) amino, (carboxyl ester) oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, substituted sulfonyloxy, thioacyl, thiol, alkylthio, and substituted alkylthio, wherein said substituents are as defined herein.

“Substituted alkenyl” refers to alkenyl groups having from 1 to 3 substituents, and preferably 1 to 2 substituents, selected from the group consisting of alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester) amino, (carboxyl ester) oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxyl, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, substituted sulfonyloxy, thioacyl, thiol, alkylthio, and substituted alkylthio, wherein said substituents are as defined herein and with the proviso that any hydroxyl or thiol substitution is not attached to a vinyl (unsaturated) carbon atom.

“Heteroalkenyl” refers to an alkenyl group where one or more carbons is replaced with one or more —O—, —S—, SO₂, P containing moiety, or —NR^{Q}— moieties where R^{Q} is H or C₁-C₆ alkyl. Substituted heteroalkenyl refers to a heteroalkenyl group having from 1 to 5, preferably 1 to 3, or more preferably 1 to 2 substituents selected from the group consisting of alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester) amino, (carboxyl ester) oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, substituted sulfonyloxy, thioacyl, thiol, alkylthio, and substituted alkylthio, wherein said substituents are as defined herein.

“Substituted alkynyl” refers to alkynyl groups having from 1 to 3 substituents, and preferably 1 to 2 substituents, selected from the group consisting of alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester) amino, (carboxyl ester) oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, substituted sulfonyloxy, thioacyl, thiol, alkylthio, and substituted alkylthio, wherein said substituents are as defined herein and with the proviso that any hydroxyl or thiol substitution is not attached to an acetylenic carbon atom.

“Heteroalkynyl” refers to an alkynyl group one or more carbons is replaced with —O—, —S—, SO₂, P containing moiety, or —NR^{Q}— moieties where R^{Q} is H or C₁-C₆ alkyl. Substituted heteroalkynyl refers to a heteroalkynyl group having from 1 to 5, preferably 1 to 3, or more preferably 1 to 2 substituents selected from the group consisting of alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester) amino, (carboxyl ester) oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, substituted sulfonyloxy, thioacyl, thiol, alkylthio, and substituted alkylthio, wherein said substituents are as defined herein.

“Alkylene” refers to divalent saturated aliphatic hydrocarbyl groups having from 1 to 10 carbon atoms, preferably having from 1 to 6 and more preferably 1 to 3 carbon atoms that are either straight-chained- or branched. Higher carbon atom containing alkenyl groups are also contemplated in certain embodiments, as the context will indicate. This term is exemplified by groups such as methylene (—CH₂—), ethylene (—CH₂CH₂—), n-propylene (—CH₂CH₂CH₂—), iso-propylene (—CH₂CH(CH₃)— or —CH(CH₃)CH₂—), butylene (—CH₂CH₂CH₂CH₂—), isobutylene (—CH₂CH(CH₃)CH₂—), sec-butylene (—CH₂CH₂(CH₃)CH), and the like. Similarly, “alkenylene” and “alkynylene” refer to an alkylene moiety containing respective 1 or 2 carbon-carbon double bonds or a carbon-carbon triple bond.

“Substituted alkylene” refers to an alkylene group having from 1 to 3 hydrogens replaced with substituents selected from the group consisting of alkyl, substituted alkyl, alkoxy, substituted alkoxy, acyl, acylamino, acyloxy, amino, substituted amino, aminoacyl, aryl, substituted aryl, aryloxy, substituted aryloxy, cyano, halogen, hydroxyl, nitro, carboxyl, carboxyl ester, cycloalkyl, substituted cycloalkyl, heteroaryl, substituted heteroaryl, heterocyclic, substituted heterocyclic, and oxo wherein said substituents are defined herein. In some embodiments, the alkylene has 1 to 2 of the aforementioned groups, or having from 1-3 carbon atoms replaced with —O—, —S—, SO₂, P containing moiety or —NR^{Q}— moieties where R^{Q} is H or C₁-C₆ alkyl. It is to be noted that when the alkylene is substituted by an oxo group, 2 hydrogens attached to the same carbon of the alkylene group are replaced by “═O.” “Substituted alkenylene” and “substituted alkynylene” refer to alkenylene and alkynylene moieties substituted with substituents as described for substituted alkylene.

“Alkynylene” refers to straight or branched divalent hydrocarbyl groups having from 2 to 10 carbon atoms and preferably 2 to 6 carbon atoms or preferably 2 to 3 carbon atoms and having at least 1 and preferably from 1 to 2 sites of acetylenic (—C≡C—) unsaturation. Higher carbon atom containing alkynylene groups are also contemplated in certain embodiments, as the context will indicate. Examples of such alkynylene groups include —C≡C— and —CH₂C≡C—.

“Substituted alkynylene” refers to alkynylene groups having from 1 to 3 substituents, and preferably 1 to 2 substituents, selected from the group consisting of alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester) amino, (carboxyl ester) oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, substituted sulfonyloxy, thioacyl, thiol, alkylthio, and substituted alkylthio, wherein said substituents are as defined herein and with the proviso that any hydroxyl or thiol substitution is not attached to an acetylenic carbon atom.

“Heteroalkylene” refers to an alkylene group wherein one or more carbons is replaced with —O—, —S—, SO₂, a P containing moiety, or —NR^{Q}— moieties where R^{Q} is H or C₁-C₆ alkyl. “Substituted heteroalkylene” refers to heteroalkynylene groups having from 1 to 3 substituents, and preferably 1 to 2 substituents, selected from the substituents disclosed for substituted alkylene.

“Heteroalkenylene” refers to an alkenylene group wherein one or more carbons is replaced with —O—, —S—, SO₂, a P containing moiety, or —NR^{Q}— moieties where R^{Q} is H or C₁-C₆ alkyl. “Substituted heteroalkenylene” refers to heteroalkynylene groups having from 1 to 3 substituents, and preferably 1 to 2 substituents, selected from the substituents disclosed for substituted alkenylene.

“Heteroalkynylene” refers to an alkynylene group wherein one or more carbons is replaced with —O—, —S—, SO₂, a P containing moiety, or —NR^{Q}— moieties where R^{Q} is H or C₁-C₆ alkyl. “Substituted heteroalkynylene” refers to heteroalkynylene groups having from 1 to 3 substituents, and preferably 1 to 2 substituents, selected from the substituents disclosed for substituted alkynylene.

“Alkoxy” refers to the group —O-alkyl wherein alkyl is defined herein. Alkoxy includes, by way of example, methoxy-, ethoxy, n-propoxy, isopropoxy, n-butoxy, t-butoxy, -sec-butoxy, and-n-pentoxy.

“Substituted alkoxy” refers to the group —O-(substituted alkyl) wherein substituted alkyl is defined herein.

“Acyl” refers to the groups H—C(O), -alkyl-C—(O)—, substituted alkyl-C(O)—, alkenyl-C(O)—, substituted alkenyl-C(O)—, alkynyl-C(O)—, substituted alkynyl-C(O)—, cycloalkyl-C(O)—, substituted cycloalkyl-C(O)—, cycloalkenyl-C(O)—, substituted cycloalkenyl-C(O)—, aryl-C(O)—, substituted aryl-C(O)—, heteroaryl-C(O)—, substituted heteroaryl-C(O)—, heterocyclic-C(O), and substituted-heterocyclic-C—(O)—, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. Acyl includes the “acetyl” group CH₃C(O)—.

“Acylamino” refers to the groups —NR⁴⁰C(O) alkyl, —NR⁴⁰C(O) substituted alkyl, —NR⁴⁰C(O) cycloalkyl, —NR⁴⁰C(O) substituted cycloalkyl, —NR⁴⁰C(O) cycloalkenyl, —NR⁴⁰C(O) substituted cycloalkenyl, —NR⁴⁰C(O) alkenyl, —NR⁴⁰C(O) substituted alkenyl, —NR⁴⁰C(O) alkynyl, —NR⁴⁰C(O) substituted alkynyl, —NR⁴⁰C(O) aryl, —NR⁴⁰C(O) substituted aryl, —NR⁴⁰C(O) heteroaryl, —NR⁴⁰C(O) substituted heteroaryl, —NR⁴⁰C(O) heterocyclic, and —NR⁴⁰C(O) substituted heterocyclic wherein R⁴⁰ is hydrogen or alkyl and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Acyloxy” refers to the groups alkyl-C—(O)O, substituted-alkyl-C—(O)O—, alkenyl-C(O)O—, substituted alkenyl-C(O)O—, alkynyl-C(O)O—, substituted alkynyl-C(O)O—, aryl-C(O)O, substituted-aryl-C—(O)O—, cycloalkyl-C(O)O—, substituted cycloalkyl-C(O)O—, cycloalkenyl-C(O)O—, substituted cycloalkenyl-C(O)O—, heteroaryl-C(O)O—, substituted heteroaryl-C(O)O,-heterocyclic-C—(O)O, and substituted-heterocyclic-C—(O)O— wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Amino” refers to the group —NH₂.

“Substituted amino” refers to the group —NR⁴¹R⁴² where R⁴¹ and R⁴⁰ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, substituted heterocyclic, —SO₂-alkyl, —SO₂-substituted alkyl, —SO₂-alkenyl, —SO₂-substituted alkenyl, —SO₂-cycloalkyl, —SO₂-substituted cycloalkyl, —SO₂-cycloalkenyl, —SO₂-substituted cylcoalkenyl, —SO₂-aryl, —SO₂-substituted aryl, —SO₂-heteroaryl, —SO₂-substituted heteroaryl, —SO₂-heterocyclic, and —SO₂-substituted heterocyclic and wherein R⁴¹ and R⁴² are optionally joined, together with the nitrogen bound thereto to form a heterocyclic or substituted heterocyclic group, provided that R⁴¹ and R⁴² are both not hydrogen, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. When R⁴¹ is hydrogen and R⁴² is alkyl, the substituted amino group is sometimes referred to herein as alkylamino. When R⁴¹ and R⁴² are alkyl, the substituted amino group is sometimes referred to herein as dialkylamino. When referring to a monosubstituted amino, it is meant that either R⁴¹ or R⁴² is hydrogen but not both. When referring to a disubstituted amino, it is meant that neither R⁴¹ nor R⁴² are hydrogen.

“Aminocarbonyl” refers to the group —C(O)NR⁵⁰R⁵¹ where R⁵⁰ and R⁵¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R⁵⁰ and R⁵¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminothiocarbonyl” refers to the group —C(S)NR⁵⁰R⁵¹ where R⁵⁰ and R⁵¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R⁵⁰ and R⁵¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminocarbonylamino” refers to the group —NR⁴⁰C(O) NR⁵⁰R⁵¹ where R⁴⁰ is hydrogen or alkyl and R⁵⁰ and R⁵¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic, and where R⁵⁰ and R⁵¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminothiocarbonylamino” refers to the group —NR⁴⁰C(S)NR⁵⁰R⁵¹ where R⁴⁰ is hydrogen or alkyl and R⁵⁰ and R⁵¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R⁵⁰ and R⁵¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminocarbonyloxy” refers to the group —O—C(O) NR⁵⁰R⁵¹ where R⁵⁰ and R⁵¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R⁵⁰ and R⁵¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminosulfonyl” refers to the group —SO₂NR⁵⁰R⁵¹ where R⁵⁰ and R⁵¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R⁵⁰ and R⁵¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminosulfonyloxy” refers to the group —O—SO₂NR⁵⁰R⁵¹ where R⁵⁰ and R⁵¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R⁵⁰ and R⁵¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminosulfonylamino” refers to the group —NR⁴⁰SO₂NR⁵⁰R⁵¹ where R⁴⁰ is hydrogen or alkyl and R⁵⁰ and R⁵¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R⁵⁰ and R⁵¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Amidino” refers to the group —C(═NR⁵²) NR⁵⁰R⁵¹ where R⁵⁰, R⁵¹, and R⁵² are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R⁵⁰ and R⁵¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aryl” or “Ar” refers to an aromatic carbocyclic group of from 6 to 14 carbon atoms having a single ring (e.g., phenyl) or multiple condensed rings (e.g., naphthyl or anthryl) which condensed rings may or may not be aromatic (e.g., 2-benzoxazolinone, 2H-1,4-benzoxazin-3 (4H)-one-7-yl, and the like) provided that the point of attachment is at an aromatic carbon atom. Certain, preferred aryl groups include phenyl and naphthyl.

“Substituted aryl” refers to aryl groups which are substituted with 1 to 5, preferably 1 to 3, or more preferably 1 to 2 substituents selected from the group consisting of alkyl, substituted alkyl, alkenyl, substituted alkenyl, alkynyl, substituted alkynyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester) amino, (carboxyl ester) oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, substituted sulfonyloxy, thioacyl, thiol, alkylthio, and substituted alkylthio, wherein said substituents are as defined herein.

“Arylene” refers to a divalent aromatic carbocyclic group of from 6 to 14 carbon atoms having a single ring or multiple condensed rings. “Substituted arylene” refers to an arylene having from 1 to 5, preferably 1 to 3, or more preferably 1 to 2 substituents as defined for aryl groups.

“Heteroarylene” refers to a divalent aromatic group of from 1 to 10 carbon atoms and 1 to 4 heteroatoms selected from the group consisting of oxygen, nitrogen and sulfur within the ring. “Substituted heteroarylene” refers to heteroarylene groups that are substituted with from 1 to 5, preferably 1 to 3, or more preferably 1 to 2 substituents selected from the group consisting of the same group of substituents defined for substituted aryl. Unless otherwise noted, the context will clearly indicate, whether an aryl or heteroaryl moiety is monovalent or divalent.

“Aryloxy” refers to the group —O-aryl-, where aryl is as defined herein, that includes, by way of example, phenoxy and naphthoxy.

“Substituted aryloxy” refers to the group —O-(substituted aryl) where substituted aryl is as defined herein.

“Arylthio” refers to the group —S-aryl-, where aryl is as defined herein.

“Substituted arylthio” refers to the group —S-(substituted aryl), where substituted aryl is as defined herein.

“Carbonyl” refers to the divalent group —C(O)-which is equivalent to —C(═O)—.

“Carboxyl” or “carboxy” refers to —COOH or salts thereof.

“Carboxyl ester” or “carboxy ester” refers to the group —C(O)(O)-alkyl, —C(O)(O)-substituted alkyl, —C(O)O-alkenyl, —C(O)(O)-substituted alkenyl, —C(O)(O)-alkynyl, —C(O)(O)-substituted alkynyl, —C(O)(O)-aryl, —C(O)(O)-substituted-aryl, —C(O)(O)-cycloalkyl, —C(O)(O)-substituted cycloalkyl, —C(O)(O)-cycloalkenyl, —C(O)(O)-substituted cycloalkenyl, —C(O)(O)-heteroaryl, —C(O)(O)-substituted heteroaryl, —C(O)(O)-heterocyclic, and —C(O)(O)-substituted heterocyclic wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“(Carboxyl ester) amino” refers to the group —NR⁴⁰C(O)(O)-alkyl, —NR⁴⁰C(O)(O)-substituted alkyl, —NR⁴⁰C(O)O-alkenyl, —NR⁴⁰C(O)(O)-substituted alkenyl, —NR⁴⁰C(O)(O)-alkynyl, —NR⁴⁰C(O)(O)-substituted alkynyl, —NR⁴⁰C(O)(O)-aryl, —NR⁴⁰C(O)(O)-substituted-aryl, —NR⁴⁰C(O)(O)-cycloalkyl, —NR⁴⁰C(O)(O)-substituted cycloalkyl, —NR⁴⁰C(O)(O)-cycloalkenyl, —NR⁴⁰C(O)(O)-substituted cycloalkenyl, —NR⁴⁰C(O)(O)-heteroaryl, —NR⁴⁰C(O)(O)-substituted heteroaryl, —NR⁴⁰C(O)(O)-heterocyclic, and —NR⁴⁰C(O)(O)-substituted heterocyclic wherein R⁴⁰ is alkyl or hydrogen, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“(Carboxyl ester) oxy” refers to the group —O—C(O)O-alkyl, —O—C(O)O-substituted alkyl, —O—C(O)O-alkenyl, —O—C(O)O-substituted alkenyl, —O—C(O)O-alkynyl, —O—C(O)(O)-substituted alkynyl, —O—C(O)O-aryl, —O—C(O)O-substituted-aryl, —O—C(O)O-cycloalkyl, —O—C(O)O-substituted cycloalkyl, —O—C(O)O-cycloalkenyl, —O—C(O)O-substituted cycloalkenyl, —O—C(O)O-heteroaryl, —OC(O)—O-substituted heteroaryl, —OC(O)—O-heterocyclic-, and —OC(O)—O-substituted heterocyclic wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Cyano” refers to the group —CN.

“Cycloalkyl” refers to cyclic alkyl groups of from 3 to 10 carbon atoms having single or multiple cyclic rings including fused, bridged, and spiro ring systems, and further includes cycloalkenyl. The fused ring can be an aryl ring provided that the non aryl part is joined to the rest of the molecule. Examples of suitable cycloalkyl groups include, for instance, adamantyl, cyclopropyl, cyclobutyl, cyclopentyl, and cyclooctyl.

“Cycloalkenyl” refers to nonaromatic-cyclic alkyl groups of from 3 to 10 carbon atoms having single or multiple cyclic rings and having at least one >C═C< ring unsaturation and preferably from 1 to 2 sites of >C═C< ring unsaturation.

“Substituted cycloalkyl” and “substituted cycloalkenyl” refers to a cycloalkyl or cycloalkenyl group having from 1 to 5 or preferably 1 to 3 substituents selected from the group consisting of oxo, thioxo, alkyl, substituted alkyl, alkenyl, substituted alkenyl, alkynyl, substituted alkynyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester) amino, (carboxyl ester) oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, substituted sulfonyloxy, thioacyl, thiol, alkylthio, and substituted alkylthio, wherein said substituents are as defined herein.

“Cycloalkyloxy” refers to —O-cycloalkyl-.

“Substituted cycloalkyloxy refers to —O-(substituted cycloalkyl).

“Cycloalkylthio” refers to —S-cycloalkyl-.

“Substituted cycloalkylthio” refers to —S-(substituted cycloalkyl).

“Cycloalkenyloxy” refers to —O-cycloalkenyl-.

“Substituted cycloalkenyloxy” refers to —O-(substituted cycloalkenyl).

“Cycloalkenylthio” refers to —S-cycloalkenyl-.

“Substituted cycloalkenylthio” refers to —S-(substituted cycloalkenyl).

“Guanidino” refers to the group —NHC(═NH)NH₂.

Substituted guanidino” refers to —NR⁵³C(═NR⁵³)N(R⁵³)₂ where each R⁵³ is independently selected from the group consisting of hydrogen, alkyl, substituted alkyl, aryl, substituted aryl, heteroaryl, substituted heteroaryl, cycloalkyl, substituted cycloalkyl, heterocyclic, and substituted heterocyclic and two R⁵³ groups attached to a common guanidino nitrogen atom are optionally joined together with the nitrogen bound thereto to form a heterocyclic or substituted heterocyclic group, provided that at least one R⁵³ is not hydrogen, and wherein said substituents are as defined herein.

“Halo” or “halogen” refers to fluoro, chloro, bromo and iodo.

“Hydroxy” or “hydroxyl” refers to the group —OH.

“Heteroaryl” refers to an aromatic group of from 1 to 10 carbon atoms and 1 to 4 heteroatoms selected from the group consisting of oxygen, nitrogen and sulfur within the ring. Such heteroaryl groups can have a single ring (e.g., pyridinyl or furyl) or multiple condensed rings (e.g., indolizinyl or benzothienyl) wherein the condensed rings may or may not be aromatic and/or contain a heteroatom provided that the point of attachment is through an atom of the aromatic heteroaryl group. In one embodiment, the nitrogen and/or the sulfur ring atom(s) of the heteroaryl group are optionally oxidized to provide for the N-oxide (N→O), sulfinyl, or sulfonyl moieties. Certain non-limiting examples include pyridinyl, pyrrolyl, indolyl, thiophenyl, oxazolyl, thizolyl, and -furanyl.

“Substituted heteroaryl” refers to heteroaryl groups that are substituted with from 1 to 5, preferably 1 to 3, or more preferably 1 to 2 substituents selected from the group consisting of the same group of substituents defined for substituted aryl.

“Heteroaryloxy” refers to —O-heteroaryl.

“Substituted heteroaryloxy” refers to the group —O-(substituted heteroaryl).

“Heteroarylthio” refers to the group —S-heteroaryl-.

“Substituted heteroarylthio” refers to the group —S-(substituted heteroaryl).

“Heterocycle” or “heterocyclic” or “heterocycloalkyl” or “heterocyclyl” refers to a saturated or partially saturated, but not aromatic, group having from 1 to 10 ring carbon atoms and from 1 to 4 ring heteroatoms selected from the group consisting of nitrogen, sulfur, or oxygen. Heterocycle encompasses single ring or multiple condensed rings, including fused bridged and spiro ring systems. In fused ring systems, one or more the rings can be cycloalkyl, aryl, or heteroaryl provided that the point of attachment is through a nonaromatic ring. In one embodiment, the nitrogen and/or sulfur atom(s) of the heterocyclic group are optionally oxidized to provide for the —N-oxide, sulfinyl-, or sulfonyl moieties.

“Substituted heterocyclic” or “substituted heterocycloalkyl” or “substituted heterocyclyl” refers to heterocyclyl groups that are substituted with from 1 to 5 or preferably 1 to 3 of the same substituents as defined for substituted cycloalkyl.

“Heterocyclyloxy” refers to the group —O-heterocycyl.

“Substituted heterocyclyloxy” refers to the group —O-(substituted heterocycyl).

“Heterocyclylthio” refers to the group —S-heterocycyl.

“Substituted heterocyclylthio” refers to the group —S-(substituted heterocycyl).

Examples of heterocycle and heteroaryls include, but are not limited to, azetidine, pyrrole, furan, thiophene, imidazole, pyrazole, pyridine, pyrazine, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4-tetrahydroisoquinoline, 4,5,6,7-tetrahydrobenzo-[b]thiophene, thiazole, thiazolidine, thiophene, benzo[b]thiophene, morpholinyl, thiomorpholinyl (also referred to as thiamorpholinyl), 1,1-dioxothiomorpholinyl, piperidinyl, pyrrolidine, and tetrahydrofuranyl.

“Nitro” refers to the group —NO₂.

“Oxo” refers to the atom (═O).

Phenylene refers to a divalent aryl ring, where the ring contains 6 carbon atoms.

Substituted phenylene refers to phenylenes which are substituted with 1 to 4, preferably 1 to 3, or more preferably 1 to 2 substituents selected from the group consisting of alkyl, substituted alkyl, alkenyl, substituted alkenyl, alkynyl, substituted alkynyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester) amino, (carboxyl ester) oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, substituted sulfonyloxy, thioacyl, thiol, alkylthio, and substituted alkylthio, wherein said substituents are as defined herein.

“Spirocycloalkyl” and “spiro ring systems” refers to divalent cyclic groups from 3 to 10 carbon atoms having a cycloalkyl or heterocycloalkyl ring with a spiro union (the union formed by a single atom which is the only common member of the rings).

“Sulfonyl” refers to the divalent group —S(O)₂—.

“Substituted sulfonyl” refers to the group —SO₂-alkyl-, —SO₂-substituted-alkyl, —SO₂-alkenyl, —SO₂-substituted alkenyl, —SO₂-cycloalkyl, —SO₂-substituted cycloalkyl, —SO₂-cycloalkenyl, —SO₂-substituted cylcoalkenyl, —SO₂-aryl, —SO₂-substituted aryl, —SO₂-heteroaryl, —SO₂-substituted heteroaryl, —SO₂-heterocyclic, —SO₂-substituted-heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. Substituted sulfonyl includes groups such as methyl-SO₂—, phenyl-SO₂—, and 4-methylphenyl-SO₂—.

“Substituted sulfonyloxy” refers to the group —OSO₂-alkyl, —OSO₂-substituted-alkyl, —OSO₂-alkenyl, —OSO₂-substituted alkenyl, —OSO₂-cycloalkyl, —OSO₂-substituted cycloalkyl, —OSO₂-cycloalkenyl, —OSO₂-substituted cylcoalkenyl, —OSO₂-aryl, —OSO₂-substituted aryl, —OSO₂-heteroaryl, —OSO₂-substituted heteroaryl, —OSO₂-heterocyclic, —OSO₂-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Thioacyl” refers to the groups H—C(S)—, alkyl-C(S)—, substituted alkyl-C(S),-alkenyl-C—(S), substituted-alkenyl-C—(S)—, alkynyl-C(S)—, substituted alkynyl-C(S)—, cycloalkyl-C(S)—, substituted cycloalkyl-C(S)—, cycloalkenyl-C(S)—, substituted cycloalkenyl-C(S)—, aryl-C(S)—, substituted aryl-C(S)—, heteroaryl-C(S)—, substituted heteroaryl-C(S)—, heterocyclic-C(S), and substituted-heterocyclic-C—(S)—, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Thiol” refers to the group —SH.

“Thiocarbonyl” refers to the divalent group —C(S)— which is equivalent to —C(═S)—.

“Thioxo” refers to the atom (═S).

“Alkylthio” refers to the group —S-alkyl-wherein alkyl is as defined herein.

“Substituted alkylthio” refers to the group —S-(substituted alkyl) wherein substituted alkyl is as defined herein.

“Optionally substituted” refers to a group selected from that group and a substituted form of that group. Substituents are such as those defined hereinabove. E.g., and without limitation, substituents can be selected from monovalent and divalent groups, such as, C₁-C₁₀ or C₁-C₆ alkyl, substituted C₁-C₁₀ or C₁-C₆ alkyl, C₂-C₆ alkenyl, C₂-C₆ alkynyl, C₆-C₁₀ aryl, C₃-C₈ cycloalkyl, C₂-C₁₀ heterocyclyl, C₁-C₁₀ heteroaryl, substituted C₂-C₆ alkenyl, substituted C₂-C₆ alkynyl, substituted C₆-C₁₀ aryl, substituted C₃-C₈ cycloalkyl, substituted C₂-C₁₀ heterocyclyl, substituted C₁-C₁₀ heteroaryl, halo, nitro, cyano, oxo (═O), —CO₂H or a C₁-C₆ alkyl ester thereof.

Unless indicated otherwise, the nomenclature of substituents that are not explicitly defined herein are arrived at by naming the terminal portion of the functionality followed by the adjacent functionality toward the point of attachment. For example, the substituent “alkoxycarbonylalkyl” refers to the group (alkoxy)-C(O)-(alkyl)-.

It is understood that in all substituted groups defined above, polymers arrived at by defining substituents with further substituents to themselves (e.g., substituted aryl having a substituted aryl group as a substituent which is itself substituted with a substituted aryl group, etc.) are not intended for inclusion herein. In such cases, the maximum number of such substituents is three. That is to say that each of the above definitions is constrained by a limitation that, for example, substituted aryl groups are limited to-substituted aryl-(substituted aryl)-substituted aryl.

It is understood that the above definitions are not intended to include impermissible substitution patterns (e.g., methyl substituted with 5 fluoro groups). Such impermissible substitution patterns are well known to the skilled artisan.

A “salt” is derived from a variety of organic and inorganic counter ions well known in the art and include, when the compound has an acidic functionality, by way of example only, sodium, potassium, calcium, magnesium, ammonium, and tetraalkylammonium; and when the molecule has a basic functionality, salts of organic or inorganic acids, such as hydrochloride, hydrobromide, tartrate, mesylate, acetate, maleate, and oxalate. Salts include acid addition salts formed with inorganic acids or organic acids. Inorganic acids suitable for forming acid addition salts include, by way of example and not limitation, hydrohalide acids (e.g., hydrochloric acid, hydrobromic acid, hydroiodic acid, etc.), sulfuric acid, nitric acid, phosphoric acid, and the like.

Organic acids suitable for forming acid addition salts include, by way of example and not limitation, acetic acid, trifluoroacetic acid, propionic acid, hexanoic acid, cyclopentanepropionic acid, glycolic acid, oxalic acid, pyruvic acid, lactic acid, malonic acid, succinic acid, malic acid, maleic acid, fumaric acid, tartaric acid, citric acid, palmitic acid, benzoic acid, 3-(4-hydroxybenzoyl) benzoic acid, cinnamic acid, mandelic acid, alkylsulfonic acids (e.g., methanesulfonic acid, ethanesulfonic acid, 1,2-ethane-disulfonic acid, 2-hydroxyethanesulfonic acid, etc.), arylsulfonic acids (e.g., benzenesulfonic acid, 4-chlorobenzenesulfonic acid, 2-naphthalenesulfonic acid, 4-toluenesulfonic acid, camphorsulfonic acid, etc.), glutamic acid, hydroxynaphthoic-acid, salicylic acid, stearic acid, muconic acid, and the like.

Salts also include salts formed when an acidic proton present in the parent compound is either replaced by a metal ion (e.g., an alkali metal ion, an alkaline earth metal ion, or an aluminum ion) or by an ammonium ion (e.g., an ammonium ion derived from an organic base, such as, ethanolamine, diethanolamine, triethanolamine, morpholine, piperidine, dimethylamine, diethylamine, triethylamine, and ammonia).

Amino acids in a protein coding sequence are identified herein by the following abbreviations and symbols. Specific amino acids are identified by a three-letter abbreviation, as follows: Ala is alanine, Arg is arginine, Asn is asparagine, Asp is aspartic acid, Cys is cysteine, Gln is glutamine, Glu is glutamic acid, Gly is glycine, His is histidine, Leu is leucine, Ile is isoleucine, Lys is lysine, Met is methionine, Phe is phenylalanine, Pro is proline, Ser is serine, Thr is threonine, Trp is tryptophan, Tyr is tyrosine, and Val is valine, or by a one-letter abbreviation, as follows: A is alanine, R is arginine, N is asparagine, D is aspartic acid, C is cysteine, Q is glutamine, E is glutamic acid, G is glycine, H is histidine, L is leucine, I is isoleucine, K is lysine, O is pyrrolysine, M is methionine, F is phenylalanine, P is proline, S is serine, T is threonine, W is tryptophan, Y is tyrosine, and V is valine. A dash (−) in a consensus sequence indicates that there is no amino acid at the specified position. A plus (+) in a consensus sequence indicates any amino acid may be present at the specified position. Thus, a plus in a consensus sequence herein indicates a position at which the amino acid is generally non-conserved; a homologous enzyme sequence, when aligned with the consensus sequence, can have any amino acid at the indicated “+” position. Specific amino acids in a protein coding sequence are identified by their respective one-letter abbreviation followed by the amino acid position in the protein coding sequence where 1 corresponds to the amino acid (typically methionine) at the N-terminus of the protein. For example, G204 in C. sativa wild type OLS refers to the glycine at position 204 from the OLS N-terminal methionine (i.e., M1). Amino acid substitutions (i.e., point mutations) are indicated by identifying the mutated (i.e., progeny) amino acid after the one-letter code and number in the parental protein coding sequence; for example, G204A in C. sativa OLS refers to substitution of glycine by alanine at position 204 in the OLS protein coding sequence. The mutation may also be identified in parentheticals, for example OLS (G204A). Multiple point mutations in the protein coding sequence are separated by a backslash (/); for example, OLS G204A/Q205N indicates that mutations G204A and Q205N are both present in the OLS protein coding sequence. The number of mutations introduced into some examples has been annotated by a dash followed by the number of mutations, preceding the parenthetical identification of the mutation (e.g., B1Q2B6-1 (G204A)). The Uniprot IDs with and without the dash and number are used interchangeably herein (i.e., B1Q2B6-1 (G204A)=B1Q2B6 (G204A)).

As used herein, the term “express”, when used in connection with a nucleic acid encoding an enzyme or an enzyme itself in a cell, means that the enzyme, which may be an endogenous or exogenous (heterologous) enzyme, is produced in the cell. The term “overexpress”, in these contexts, means that the enzyme is produced at a higher level, i.e., enzyme levels are increased, as compared to the wild type, in the case of an endogenous enzyme. Those skilled in the art appreciate that overexpression of an enzyme can be achieved by increasing the strength or changing the type of the promoter used to drive expression of a coding sequence, increasing the strength of the ribosome binding site or Kozak sequence, increasing the stability of the mRNA transcript, altering the codon usage, increasing the stability of the enzyme, and the like.

The term “expression vector” or “vector” refer to a nucleic acid and/or a composition comprising a nucleic acid that can be introduced into a host cell, e.g., by transduction, transformation, or infection, such that the cell then produces (“expresses”) nucleic acids and/or proteins other than those native to the cell, or in a manner not native to the cell, that are contained in or encoded by the nucleic acid so introduced. Thus, an “expression vector” contains nucleic acids (ordinarily DNA) to be expressed by the host cell. Optionally, the expression vector can be contained in materials to aid in achieving entry of the nucleic acid into the host cell, such as the materials associated with a virus, liposome, protein coating, or the like. Expression vectors suitable for use in various aspects and embodiments include those into which a nucleic acid sequence can be, or has been, inserted, along with any preferred or required operational elements. Thus, an expression vector can be transferred into a host cell and, typically, replicated therein (although, one can also employ, in some embodiments, non-replicable vectors that provide for “transient” expression). In some embodiments, an expression vector that integrates into chromosomal, mitochondrial, or plastid DNA is employed. In other embodiments, an expression vector that replicates extrachromosomally is employed. Typical expression vectors include plasmids, and expression vectors typically contain the operational elements required for transcription of a nucleic acid in the vector. Such plasmids, as well as other expression vectors, are described herein or are well known to those of ordinary skill in the art.

The terms “ferment”, “fermentative”, and “fermentation” are used herein to describe culturing host cells and microbes under conditions to produce useful chemicals, including but not limited to conditions under which microbial growth, be it aerobic or anaerobic, occurs.

The term “heterologous” as used herein refers to a material that is non-native to a cell. For example, a nucleic acid is heterologous to a cell, and so is a “heterologous nucleic acid” with respect to that cell, if at least one of the following is true: (a) the nucleic acid is not naturally found in that cell (that is, it is an “exogenous” nucleic acid); (b) the nucleic acid is naturally found in a given host cell (that is, “endogenous to”), but the nucleic acid or the RNA or protein resulting from transcription and translation of this nucleic acid is produced or present in the host cell in an unnatural (e.g., greater or lesser than naturally present) amount; (c) the nucleic acid comprises a nucleotide sequence that encodes a protein endogenous to a host cell but differs in sequence from the endogenous nucleotide sequence that encodes that same protein (having the same or substantially the same amino acid sequence), typically resulting in the protein being produced in a greater amount in the cell, or in the case of an enzyme, producing a mutant version possessing altered (e.g., higher or lower or different) activity; and/or (d) the nucleic acid comprises two or more nucleotide sequences that are not found in the same relationship to each other in the cell. As another example, a protein is heterologous to a host cell if it is produced by translation of RNA or the corresponding RNA is produced by transcription of a heterologous nucleic acid; a protein is also heterologous to a host cell if it is a mutated version of an endogenous protein, and the mutation was introduced by genetic engineering.

The terms “host cell” and “host microorganism” are used interchangeably herein to refer to a living cell that can perform one or more steps of the cannabinoid pathway, e.g. and without limitation, converting malonyl-CoA and hexanoyl-CoA (or another acyl-CoA) to olivetol and olivetolic acid. A host cell can be (or is) transformed via insertion of an expression vector. A host microorganism or cell as described herein may be a prokaryotic cell (e.g., a microorganism of the kingdom Eubacteria) or a eukaryotic cell. As will be appreciated by one of skill in the art, a prokaryotic cell lacks a membrane-bound nucleus, while a eukaryotic cell has a membrane-bound nucleus. In certain instances, a host cell is part of a multi-cellular organism.

Polyketide synthases (PKSs) are a family of multi-domain enzymes or enzyme complexes that produce polyketides, a large class of secondary metabolites, in bacteria, fungi, plants, and a few animal lineages. The terms “polyketide synthase”, “PKS”, “olivetol synthase” (“OLS” or “OS”), “tetraketide synthase”, TKS, and olivetolic synthase as described herein or elsewhere typically refers to any enzyme capable of converting three molecules of malonyl-CoA and one molecule of hexanoyl-CoA or another acyl-CoA to olivetol or an olivetol analog. A wild type example of an OLS is the native C. sativa OLS enzyme (UniProt ID: B1Q2B6; SEQ ID NO:1).

SEQ ID NO: 1:
OLS
MNHLRAEGPASVLAIGTANPENILIQDEFPDYYFRVTKSEHMTQL
KEKFRKICDKSMIRKRNCFLNEEHLKQNPRLVEHEMQTLDARQDM
LVVEVPKLGKDACAKAIKEWGQPKSKITHLIFTSASTTDMPGADY
HCAKLLGLSPSVKRVMMYQLGCYGGGTVLRIAKDIAENNKGARVL
AVCCDIMACLFRGPSDSDLELLVGQAIFGDGAAAVIVGAEPDESV
GERPIFELVSTGQTILPNSEGTIGGHIREAGLIFDLHKDVPMLIS
NNIEKCLIEAFTPIGISDWNSIFWITHPGGKAILDKVEEKLDLKK
EKFVDSRHVLSEHGNMSSSTVLFVMDELRKRSLEEGKSTTGDGFE
WGVLFGFGPGLTVERVVVRSVPIKY

Olivetolic acid cyclase (“OAC”, EC: 4.4.1.26) is a polyketide cyclase derived from C. sativa which functions in concert with an OLS enzyme or a tetraketide synthase (“TKS”) to form OLA. See, e.g.:

SEQ ID NO: 2:
OAC
MAVKHLIVLKFKDEITEAQKEEFFKTYVNLVNIIPAMKDVYWGKD
VTQKKEEGYTHIVEVTFESVETIQDYIIHPAHVGFGDVYRSFWEK
LLIFDYTPRK

The terms “cannabinoid pathway”, “cannabinoid production”, “cannabinoid compound production”, “cannabinoid synthesis”, “THC synthesis”, and the like, refer generally to a biosynthetic pathway that facilitates the synthesis and production of olivetol, olivetolic acid, and olivetolic acid-derived compounds. This biosynthetic pathway utilizes a variety of enzymes, catalysts, and intermediate compounds. For example, cannabigerolic acid synthase (EC: 2.5.1.102) is used to convert OA to cannabigerolic acid, which is a key intermediate acted upon by a variety of enzymes during THC synthesis. Cannabidiolic acid synthase (EC: 1.21.3.7) is used to convert cannabigerolic acid into cannabidiolic acid. Tetrahydrocannabinolic acid synthase (EC: 1.21.3.8) is used to convert cannabigerolic acid into Δ⁹-tetrahydrocannabinolic acid. A cannabichromenic acid synthase is used to convert cannabigerolic acid into cannabichromenic acid (CAS #20408-52-0). These three olivetolic acid-derived compounds (i.e., cannabidiolic acid, Δ⁹-tetrahydrocannabinolic acid, and cannabichromenic acid) are themselves converted to even more diverse cannabinoids via a combination of oxidation, decarboxylation, and isomerization reactions, which can be catalyzed using either biological or synthetic catalysts, or can also occur spontaneously following heating and/or application of UV light. For example, cannabidiol results from cannabidiolic acid decarboxylation, Δ⁹-tetrahydrocannabinol results from Δ⁹-tetrahydrocannabinolic acid decarboxylation, and subsequent isomerization of Δ⁹-tetrahydrocannabinol results in Δ⁶-tetrahydrocannabinol.

As used herein, “recombinant” refers to the alteration of genetic material by human intervention. Typically, recombinant refers to the manipulation of DNA or RNA in a cell or virus or expression vector by molecular biology (recombinant DNA technology) methods, including cloning and recombination. Recombinant can also refer to manipulation of DNA or RNA in a cell or virus by random or directed mutagenesis. A “recombinant” cell or nucleic acid can typically be described with reference to how it differs from a naturally occurring counterpart (the “wild type”). In addition, any reference to a cell or nucleic acid that has been “engineered” or “modified” and variations of those terms, is intended to refer to a recombinant cell or nucleic acid.

The terms “transduce”, “transform”, “transfect”, and variations thereof as used herein refers to the introduction of one or more nucleic acids into a cell. For practical purposes, the nucleic acid must be stably maintained or replicated by the cell for a sufficient period of time to enable the function(s) or product(s) it encodes to be expressed for the cell to be referred to as “transduced”, “transformed”, or “transfected”. As will be appreciated by those of skill in the art, stable maintenance or replication of a nucleic acid may take place either by incorporation of the sequence of nucleic acids into the cellular chromosomal DNA, e.g., the genome, as occurs by chromosomal integration, or by replication extrachromosomally, as occurs with a freely-replicating plasmid. A virus can be stably maintained or replicated when it is “infective”: when it transduces a host microorganism, replicates, and (without the benefit of any complementary virus or vector) spreads progeny expression vectors, e.g., viruses, of the same type as the original transducing expression vector to other microorganisms, wherein the progeny expression vectors possess the same ability to reproduce.

### Descriptive Embodiments

In one aspect, provided herein is a process comprising:
contacting an aqueous phase comprising glucose and hexanoic acid or a salt thereof and
   an organic phase immiscible with the aqueous phase
with a heterologous microorganism comprising a Cannabis sativa olivetol synthase (which is a tetraketide synthase, csOLS), Cannabis sativa olivetolic acid cyclase (csOAC), and an acyl activating enzyme (csAAE)
to provide olivetol and olivetolic acid or a salt thereof,
wherein the olivetol and olivetolic acid is provided in a combined amount of at least about 3 g/liter over about 4 to about 7 days.

In accordance with certain embodiments of this process, other microorganisms, such as those utilized herein are useful.

In one aspect, provided herein is a process comprising:
contacting an aqueous phase comprising glucose and butyric acid or a salt thereof and
   an organic phase immiscible with the aqueous phase
with a heterologous microorganism comprising a Cannabis sativa olivetol synthase (which is a tetraketide synthase, csOLS), Cannabis sativa olivetolic acid cyclase (csOAC), and an acyl activating enzyme (csAAE)
to provide divarin and/or divarinic acid or a salt thereof.

Other microorganisms, such as those utilize herein, utilized herein are useful in accordance with certain embodiments of this process.

In one embodiment, the divarin and/or divarinic acid is provided in a combined amount of at least about 0.25-about 2 g/liter, or about 0.5-about 1 g/liter over about 4 to about 7 days.

In one embodiment, the fermenting is performed in the absence of galactose. In another embodiment, the aqueous phase comprises galactose.

In one embodiment, the fermenting is performed in the absence of galactose. In another embodiment, the aqueous phase comprises galactose.

In one aspect, provided herein is a process comprising fermenting a recombinant, heterologous fungus, to produce a compound of formula (IA) and/or (IB):

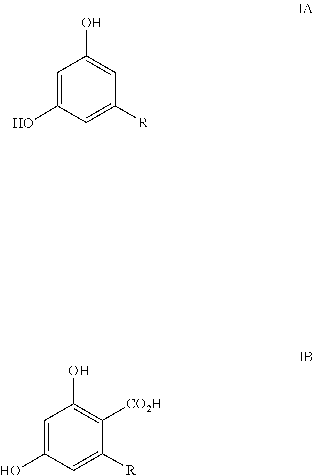

or a salt thereof wherein R is optionally substituted C₁-C₈ alkyl or optionally substituted C₂-C₆ alkenyl. In one embodiment, a compound of formula IB is produced, free of or substantially free of a compound of formula IA. In another embodiment, the fungus is a mold. In another embodiment, the fungus is a filamentous fungus.

In one embodiment, HRPKS, NRPKS and (pseudoacyl carrier protein)-thioesterase enzymes are co-expressed in the fungus. In one embodiment, the fungus is Aspergillus nidulans. Methods of fermenting Aspergillus nidulans is described in ACS Synth Biol, 2021 Aug. 20. doi: 10.1021/acssynbio.1c00309, online ahead of print, entitled “High-Titer Production of Olivetolic Acid and Analogs in Engineered Fungal Host Using a Nonplant Biosynthetic Pathway” by Ikechukwu C. Okorafor, Mengbin Chen, and Yi Tang, and supplementary materials thereto (incorporated herein in their entirety by reference); a skilled artisan can adapt from such methods to practice the present invention.

Organic Phase Immiscible with Aqueous Phase

In one embodiment, the organic phase immiscible with aqueous phase, or simply the organic phase, comprises an alkane, an alcohol with carbon number greater than 4, an ester (such as isopropyl myristate), a triglyceride (including commercially available vegetable oils such as sunflower oil, soybean oil, or olive oil), a diester (such as dialkyl malonate), a ketone, or a glyme. Other organic solvents immiscible with water or the aqueous phase employed can be utilized. In another embodiment, the organic phase comprises isopropyl myristate. Suitable solvents include without limitation, other esters, aromatic solvents, and the likes. In one embodiment, the organic phase comprises an aromatic solvent. Non limiting examples of aromatic hydrocarbon solvents include benzene, toluene, other alkylated benzenes, anisole and the likes, and mixtures thereof. In one embodiment, the organic phase comprises toluene.

In one embodiment, the organic phase immiscible with the aqueous phase comprises polypropylene glycol (PPG). In another embodiment, the PPG has an average molecular weight of greater that about 1,200. In another embodiment, the PPG has an average molecular weight of about 1,200. In another embodiment, the PPG has an average molecular weight of about 1,500. In another embodiment, the PPG has an average molecular weight of about 4,000. PPG is commercially available.

In-situ liquid-liquid extraction (biphasic fermentation) is a strategy that can be employed in accordance with the present invention for physical separation of product from microorganisms via partitioning into the water immiscible organic liquid phase from an aqueous culture phase. The organic liquid phase or organic phase is present as either an overlay if its density is less than that of the aqueous phase, or an underlay if its density is greater than that of the aqueous phase. Certain properties of the overlay or underlay are considered for production of olivetolic acid/olivetol and other resorcinols such as of formulas IA and IB: (1) non-toxic or low toxicity for growth of the host strain, and/or (2) a favorable partitioning coefficient of the product in the organic phase vs. the aqueous phase, and/or (3) preferably a lower partition coefficient for fed hexanoic acid (for olivetolic acid/olivetol) or other fatty acid such as RCO₂H (for other resorcinols) in the organic phase vs. the aqueous phase. Additional properties of the organic phase enhance its suitability for downstream conversion, e.g. and without limitation, to cannabigerol and other cannabinoid compounds, including suitability as a solvent or co-solvent during downstream prenylation or other reactions, and boiling point if downstream separation by distillation is employed.

The performance of various classes of organic phase compounds are provided herein. Among the diesters tested, certain may be toxic to growth under the test conditions. Certain diethyl esters were toxic under the test conditions with the exception of modest growth by most strains in the presence of diethyl sebacate and diethyl diethylmalonate (with glucose only, with galactose strains appeared to exhibit substantial lag). For malonate diesters, under the test conditions, di-tert-butyl malonate supported growth of all strains with glucose addition, again appearing toxic or to induce substantial lag with galactose addition.

Increasing the dialkyl ester chain length from diethyl to diisopropyl to dibutyl in a dialkyl adipate series reduced toxicity. Growth was observed with diisopropyl adipate and no apparent toxicity observed in dibutyl adipate. Dibutyl sebacate was also completely non-inhibitory to growth and accordingly, non-toxic. In certain embodiments, the minimum non-toxic internal alkyl chain length of diethyl diesters is sebacate. In certain embodiments, shorter internal alkyl chain lengths down to adipate is possible with diisopropyl diesters.

For monoester compounds, under the test conditions, octyl acetate was toxic and for the hexanoate series, growth was only observed starting with hexyl hexanoate, which was moderately non-toxic. Isopropyl octanoate was moderately inhibitory but allowed for some growth. For the decanoate series, methyl decanoate was moderately inhibitory to growth but still allowed for growth. Texanol, a monoester alcohol (2,2,4-trimethyl-1,3-pentanediol monoisobutyrate), was inhibitory to growth under the conditions tested.

However, ethyl decanoate and higher alkyl chains were increasingly non-toxic. Both ethyl and butyl laurate were non-toxic, as well as methyl and ethyl myristate. In certain embodiments, growth-suitable monoester overlays for resorcinol or cannabinoid production include hexyl hexanoate or any higher chain length alkyl hexanoate ester, C₃ chain-length or higher (e.g., and without limitation C₆-C₈ or higher) alkyl octanoate esters, and methyl (C₁) or higher (e.g., and without limitation C₆-C₈ or higher) alkyl decanoates, laurates, or myristates.

In various embodiments, esters and diesters are employed as the organic phase in accordance with the present invention.

Fatty alcohols are mostly solids above C₁₀ saturated chain length. Decanol, a liquid, was toxic to growth under test conditions. However, oleyl alcohol supported robust growth.

In certain embodiments, longer chain length (C₁₂ or higher) unsaturated fatty alcohols can be suitable overlays supporting S. cerevisiae or another fermenting organism's growth. In various embodiments, fatty alcohols, preferably C₁₂ or higher alcohols, are employed as the organic phase in accordance with the present invention.

In certain embodiments, alkanes and paraffins support robust growth. Lack of toxicity was observed for dodecane, tetradecane, hexadecane, light and heavy paraffin oils, and isopar M. In certain embodiments, C₁₂ and higher paraffins are suitable overlays supporting S. cerevisiae or another fermenting organism's growth. In various embodiments, fatty alcohols, preferably C₁₂ or higher alcohols, are employed as the organic phase in accordance with the present invention.

Certain triacylglycerols were tested, including tricaprylin, coconut oil, and canola oil (vegetable oils having different average chain length compositions of fatty acid chains, with coconut oil being predominantly C₁₂-C₁₄ saturated fatty acids, and canola being predominantly C₁₆-C₁₈ and a mixture of saturated and unsaturated fatty acids). Tricaprylin, a synthetic oil containing three C₈ fatty acid chains, was fairly toxic, however allowed some growth of strains. In certain embodiments, coconut and canola oil were non-toxic to growth.

Mixtures of isopropyl myristate (IPM) and isopar M with different diesters-dibasic esters (DBE), diethyl sebacate, and di-tert-butyl malonate were explored to investigate if lower percentage mixtures of these compounds in non-toxic IPM or isopar M would mitigate their toxicity toward growth of a microorganism such as S. cerevisiae, as they may also advantageously alter partitioning properties of olivetolic acid, olivetol, and other analogues into the overlay and could offer advantages with alternative downstream separations processes. For example, and without limitation, a DBE, which may be toxic by itself as an underlay, was much less toxic at concentrations of between 1 and 2.5% (v/v) in IPM and especially isopar M. Di-tert-butyl malonate also exhibited much lower toxicity at 1-10% (v/v), and particularly 1-2.5% (v/v), in IPM and isopar M. In certain embodiments, mixtures of longer chain monoesters or paraffins with moderately to very toxic diesters are useful according to the present invention.

In another embodiment, the aqueous phase further comprises histidine. In another embodiment, the pH of the aqueous phase is at a pH of about 4 to about 8.

In another embodiment, the olivetol and olivetolic acid is provided in a combined amount of at least about 4 g/liter over about 4 to about 7 days. In another embodiment, the olivetol and olivetolic acid is provided in a combined amount of at least about 4.5 g/liter over about 4 to about 7 days. In another embodiment, the olivetol and olivetolic acid is provided in a combined amount of at least about 5 g/liter over about 4 to about 7 days. In another embodiment, the olivetol and olivetolic acid is provided in a combined amount of at least about 7 g/liter over about 4 to about 7 days. In another embodiment, the olivetol and olivetolic acid is provided in a combined amount of at least about 9 g/liter over about 4 to about 7 days. In another embodiment, the combined amount of olivetol and olivetolic acid provided herein, is provided over 4 days. In another embodiment, the combined amount of olivetol and olivetolic acid provided herein, is provided over 5 days. In another embodiment, the combined amount of olivetol and olivetolic acid provided herein, is provided over 6 days. In another embodiment, the combined amount of olivetol and olivetolic acid provided herein, is provided over 7 days.

In one embodiment, the fermentation is performed in a semi-continuous mode (“fill-and-draw”). In another embodiment, the fermentation is performed in a continuous mode. In one embodiment, the overall combined productivity of olivetol and olivetolic acid is greater than 0.3 g per L of total volume (including aqueous and immiscible liquid phases) per day of operation. In another embodiment, the fermentation is performed in a total volume of 15 liters or at larger volume such as 1,000 liters, 10,000 liters, 20,000 or 50,000 liters, or an even larger volume. The combined yield of O and OA obtained in such large scale fermentation performed according to the present invention over 2-7 or 4-7 days, such as over 2, 3, 4, 5, 6, or 7 days is unexpectedly high. In some embodiment, the combined amount of O/OA obtained, even in large scale fermentations, e.g., and without limitation in 10,000 liters fermentations, is about 7-about 10 g/liter. In some embodiment, the combined amount of O/OA obtained, even in large scale fermentations, e.g., and without limitation in 20,000 liters fermentations, is about 7-about 10 g/liter.

In one embodiment, the productivity of the combined olivetol and olivetolic acid or a salt thereof (O+OA) is about 3 g-about 8 g/liter/day, or about 4 g/liter/day. In one embodiment, the productivity of the combined divarin and divarinic acid or a salt thereof (D +DA) is about 2 g-about 8 g/liter/day, or about 3 g/liter/day.

In one embodiment, the functional OLS has a SEQ ID NO:1. In another embodiment, the functional OLS has an at least 95% sequence identity with SEQ ID NO:1. In another embodiment, the functional olivetolic acid cyclase has at least 50%, at least 75%, or at least 95% sequence identity to SEQ ID NO:1.

In one embodiment, the functional OAC has a SEQ ID NO:2. In another embodiment, the functional OAC has an at least 95% sequence identity with SEQ ID NO:2. In another embodiment, the functional olivetolic acid cyclase has at least 50%, at least 75%, or at least 95% sequence identity to SEQ ID NO:2. In another embodiment, the functional olivetolic acid cyclase is of SEQ ID NO:3. In another embodiment, the functional olivetolic acid cyclase has at least 50%, at least 75%, or at least 95% sequence identity to SEQ ID NO:3.

SEQ ID NO: 3
MAVKHLIVLKFKDEITEAQKEEFFKTYVNLVNIIPAMKDVYWGKD
VTQKNKEEGYTHIVEVTFESVETIQDYIIHPAHVGFGDVYRSFWE
KLLIFDYTPRK

In one embodiment, the functional AAE has a SEQ ID NO:4. In another embodiment, the functional AAE has an at least 95% sequence identity with SEQ ID NO:4.

SEQ ID NO: 4:
Cannabis sativa acyl activating enzyme (CsAAE1)
MGKNYKSLDSVVASDFIALGITSEVAETLHGRLAEIVCNYGAATP
QTWINIANHILSPDLPFSLHQMLFYGCYKDFGPAPPAWIPDPEKV
KSTNLGALLEKRGKEFLGVKYKDPISSFSHFQEFSVRNPEVYWRT
VLMDEMKISFSKDPECILRRDDINNPGGSEWLPGGYLNSAKNCLN
VNSNKKLNDTMIVWRDEGNDDLPLNKLTLDQLRKRVWLVGYALEE
MGLEKGCAIAIDMPMHVDAVVIYLAIVLAGYVVVSIADSFSAPEI
STRLRLSKAKAIFTQDHIIRGKKRIPLYSRVVEAKSPMAIVIPCS
GSNIGAELRDGDISWDYFLERAKEFKNCEFTAREQPVDAYTNILF
SSGTTGEPKAIPWTQATPLKAAADGWSHLDIRKGDVIVWPTNLG
WMMGPWLVYASLLNGASIALYNGSPLVSGFAKFVQDAKVTMLGVV
PSIVRSWKSTNCVSGYDWSTIRCFSSSGEASNVDEYLWLMGRANY
KPVIEMCGGTEIGGAFSAGSFLQAQSLSSFSSQCMGCTLYILDKN
GYPMPKNKPGIGELALGPVMFGASKTLLNGNHHDVYFKGMPTLNG
EVLRRHGDIFELTSNGYYHAHGRADDTMNIGGIKISSIEIERVCN
EVDDRVFETTAIGVPPLGGGPEQLVIFFVLKDSNDTTIDLNQLRL
SFNLGLQKKLNPLFKVTRVVPLSSLPRTATNKIMRRVLRQQFSHFE

In another embodiment, the functional AAE polypeptide comprises an amino acid sequence SEQ ID NO:5. In another embodiment, the functional AAE polypeptide comprises an amino acid sequence that has at least 50%, at least 75%, or at least 95% sequence identity to SEQ ID NO:5. In another embodiment, the functional AAE polypeptide comprises an amino acid sequence SEQ ID NO:6. In another embodiment, the functional AAE polypeptide comprises an amino acid sequence that has at least 50%, at least 75%, or at least 95% sequence identity to SEQ ID NO:6. In another embodiment, the functional AAE polypeptide comprises an amino acid sequence that is SEQ ID NO:7. In another embodiment, the functional AAE polypeptide has at least 50%, at least 75%, or at least 95% sequence identity to SEQ ID NO:7.

SEQ ID NO: 5:
Cannabis sativa acyl activating enzyme (CsAAE3)
MEKSGYGRDGIYRSLRPPLHLPNNNNLSMVSFLFRNSSSYPQKPA
LIDSETNQILSFSHFKSTVIKVSHGFLNLGIKKNDWLIYAPNSIH
FPVCFLGIIASGAIATTSNPLYTVSELSKOVKDSNPKLIITVPQL
LEKVKGFNLPTILIGPDSEQESSSDKVMTFNDLVNLGGSSGSEFP
IVDDFKQSDTAALLYSSGTTGMSKGWLTHKNFIASSLMVTMEQDL
VGEMDNVFLCFLPMFHVFGLAIITYAQLQRGNTVISARFDLEKML
KDVEKYVTHLWWPPVILALSKNSMVKFNLSSIKYIGSGAAPLGKD
LMEECSKWPYGIVAQGYGMTETCGIVSMEDIRGGKRNSGSAGMLA
SGVEAQIVSVDTLKPLPPNQLGEIWVKGPNMMQGYFNNPQATKLT
IDKKGWVHTGDLGYFDEDGHLYWDRIKELIKYKGFQVAPAELEGL
LVSHPEILDAWIPFPDAEAGEVPVAYWRSPNSSLTENDVKKFIAG
QVASFKRLRKVTFINSVPKSASGKILRRELIQKVRSNM
SEQ ID NO: 6:
Truncated Cannabis sativa acyl activating enzyme
MEKSGYGRDGIYRSLRPPLHLPNNNNLSMVSFLFRNSSSYPQKPA
LIDSETNQILSFSHFKSTVIKVSHGFLNLGIKKNDWLIYAPNSIH
FPVCFLGIIASGAIATTSNPLYTVSELSKOVKDSNPKLIITVPQL
LEKVKGFNLPTILIGPDSEQESSSDKVMTFNDLVNLGGSSGSEFP
IVDDFKQSDTAALLYSSGTTGMSKGWLTHKNFIASSLMVTMEQDL
VGEMDNVFLCFLPMFHVFGLAIITYAQLQRGNTVISARFDLEKML
KDVEKYVTHLWWPPVILALSKNSMVKFNLSSIKYIGSGAAPLGKD
LMEECSKWPYGIVAQGYGMTETCGIVSMEDIRGGKRNSGSAGMLA
SGVEAQIVSVDTLKPLPPNQLGEIWVKGPNMMQGYFNNPQATKLT
IDKKGWVHTGDLGYFDEDGHLYWDRIKELIKYKGFQVAPAELEGL
LVSHPEILDAWIPFPDAEAGEVPVAYWRSPNSSLTENDVKKFIAG
QVASFKRLRKVTFINSVPKSASGKIL.
SEQ ID NO: 7:
Escherichia coli hexanoyl-CoA synthetase
amino acid sequence
MHPTGPHLGPDVLFRESNMKVTLTFNEQRRAAYRQQGLWGDASLA
DYWQQTARAMPDKIAVVDNHGASYTYSALDHAASCLANWMLAKGI
ESGDRIAFQLPGWCEFTVIYLACLKIGAVSVPLLPSWREAELVWV
LNKCQAKMFFAPTLFKQTRPVDLILPLQNQLPQLQQIVGVDKLAP
ATSSLSLSQIIADNTSLTTAITTHGDELAAVLFTSGTEGLPKGVM
LTHNNILASERAYCARLNLTWQDVFMMPAPLGHATGFLHGVTAPF
LIGARSVLLDIFTPDACLALLEQQRCTCMLGATPFVYDLLNVLEK
QPADLSALRFFLCGGTTIPKKVARECQQRGIKLLSVYGSTESSPH
AVVNLDDPLSRFMHTDGYAAAGVEIKVVDDARKTLPPGCEGEEAS
RGPNVFMGYFDEPELTARALDEEGWYYSGDLCRMDEAGYIKITGR
KKDIIVRGGENISSREVEDILLQHPKIHDACVVAMSDERLGERSC
AYVVLKAPHHSLSLEEVVAFFSRKRVAKYKYPEHIVVIEKLPRTT
SGKIQKFLLRKDIMRRLTQDVCEEIE
SEQ ID NO: 8:
AtAAE7 (nucleotide)
atggctgcgaccaaatggcgtgacatagatgatcttccaaaaatt
cccgctaattacacagctctgaccccactctggttccttgatcgc
gctgccgttgtacaccccacgagaaaatcagttatccatggtagt
agagaatacacatggagacaaacctacgatagatgtagaagattg
gcatctgctctagctgatagatccattggtccaggatctactgtt
gctattattgctccaaacattccagctatgtacgaagctcatttc
ggtgttccaatgtgtggtgctgttttgaactgtgttaacatccgc
ttgaacgctccgactgttgctttcttgttgtctcattctcaatcc
tctgttattatggttgatcaagaattcttcactttggctgaagat
tctttgagattgatggaagaaaaggctggttcctcattcaagaga
ccattgttgattgtaattggtgatcatacttgtgctccagaatca
ttgaacagagctttgtctaagggtgcaattgaatacgaagatttc
ttggctacaggtgatccaaactacccatggcaacctccagctgat
gaatggcaatccattgctctcggttacacttctggtactaccgca
agcccaaagggtgtggttttgcatcatagaggtgcttacattatg
gctttgtctaacccattgatttggggtatgcaagatggtgctgtt
tacttgtggactttaccaatgttccattgtaacggttggtgtttc
ccatggtccttggcggttctgtctggtacttcgatttgtttgaga
caagttactgctaaggaagtttactctatgattgctaagtacaag
gttactcatttctgtgctgctccagttgttttgaacgctatcgtt
aacgctccaaaggaagatactattttgccattgccacatactgtt
catgttatgactgctggtgctgcaccaccaccatctgtgttgttc
tctatgaaccaaaagggtttcagagttgctcatacttacggtctg
tctgaaacttacggtccatctactgtttgtgcttggaagccagaa
tgggattcattgccaccagaaactcaagctaagttgaacgcaaga
caaggtgttagatacactggtatggaacaattggatgttatcgat
actcaaactggtaagcctgttccagctgatggtaagactgctggc
gaaattgttttcagaggtaacatggttatgaagggttacttgaag
aacccggaagctaacaaggaaacttttgcgggaggttggttccat
tctggtgatattgctgtcaagcatccagataactacattgaaatt
aaggatagatctaaggatgtgattatttctggtggtgaaaacatt
tcatctgttgaagttgaaaacgtcgtctaccatcatccagctgtt
ttggaggcttctgttgttgctagaccagatgaaagatggcaagaa
tctccatgtgctttcgttactttgaagtccgattacgaaaagcat
gatcaaaacaagttagctcaagatattatgaagttctgtagagaa
aagttgccagcttactgggttccaaagtctgttgtgttcggccca
ttgcctaagactgctactggtaagattcagaagcatatcttgaga
actaaggctaaggaaatgggtccagttccaagataa
SEQ ID NO: 9:
AtAAE7 (protein)
MAATKWRDIDDLPKIPANYTALTPLWFLDRAAVVHPTRKSVIHGS
REYTWRQTYDRCRRLASALADRSIGPGSTVAIIAPNIPAMYEAHF
GVPMCGAVLNCVNIRLNAPTVAFLLSHSQSSVIMVDQEFFTLAED
SLRLMEEKAGSSFKRPLLIVIGDHTCAPESLNRALSKGAIEYEDF
LATGDPNYPWQPPADEWQSIALGYTSGTTASPKGVVLHHRGAYIM
ALSNPLIWGMQDGAVYLWTLPMFHCNGWCFPWSLAVLSGTSICLR
QVTAKEVYSMIAKYKVTHFCAAPVVLNAIVNAPKEDTILPLPHTV
HVMTAGAAPPPSVLFSMNQKGFRVAHTYGLSETYGPSTVCAWKPE
WDSLPPETQAKLNARQGVRYTGMEQLDVIDTQTGKPVPADGKTAG
EIVFRGNMVMKGYLKNPEANKETFAGGWFHSGDIAVKHPDNYIEI
KDRSKDVIISGGENISSVEVENVVYHHPAVLEASVVARPDERWQE
SPCAFVTLKSDYEKHDQNKLAQDIMKFCREKLPAYWVPKSVVFGP
LPKTATGKIQKHILRTKAKEMGPVPR
SEQ ID NO: 10:
At4CLL6 (nucleotide)
atggctgccacacacttacatattccccccaatcctaagactcag
acgtcgcaccaaaacccacccttttggtttagctccaaaaccggt
atctatacatctaagtttccatcgctacacttgccagtggatcca
aacttggatgctgtttctgctttgttctcccataagcatcatggt
gatactgctttgattgattctttgactggtttctctatttcccat
actgaattgcaaattatggttcaatctatggctgctggtatttac
catgttttgggtgtgagacaaggtgatgttgtctctttggttttg
ccaaactctgtttacttcccaatgattttcttgtctttgatttct
ctcggtgcgatagttactactatgaacccatcctcttctttgggt
gaaattaagaagcaagtttctgaatgttctgttggtttagctttc
acttctactgaaaacgttgaaaagttatcttctttgggtgtttct
gttatttctgtttctgaatcttacgatttcgattctattagaatt
gaaaacccaaagttctactctattatgaaggaatccttcggtttc
gttccaaagccattgattaagcaagatgacgttgctgccattatg
tactctagcggtaccactggtgcttctaagggtgttcttcttacc
catagaaacctaattgcttctatggaattgttcgttagattcgaa
gcttctcaatacgaatacccaggttcttctaacgtttacttggct
gctttaccattgtgtcatatttacggcttgtctttgttcgttatg
ggtttgttgtctttgggtagcacgattgttgtaatgaagagattc
gatgcttcagatgttgtaaacgttatagaaagattcaagattaca
catttcccagtcgttccaccaatgttgatggctctcactaagaag
gctaagggtgtttgtggtgaagttttcaagtctttgaagcaagtt
tcttccggtgctgctccattgtctagaaagttcattgaagatttc
ttacaaactttgccacatgttgatttgattcaaggttacggtatg
actgaatcgactgctgttggtactagaggtttcaactctgaaaag
ttgtctagatactcttctgttggtttgcttgctccaaacatgcaa
gctaaggttgtcgattggtcttctggttcttttttgccaccaggt
aacagaggcgaattgtggattcaaggtccaggtgttatgaagggt
tacttgaacaacccaaaggctacccaaatgtctattgttgaagat
tcctggttgcgtactggtgatattgcttacttcgatgaagatggt
tacttgttcattgttgataggattaaggaaattattaagtacaag
ggtttccaaatagctccagcagatttggaagctgttttggtttct
catccattgatcattgatgctgctgttactgctgcgcctaacgaa
gaatgtggtgaaattccagttgctttcgttgttagacgacaagaa
accactttatcagaagaagatgttattagctacgttgcttctcaa
gttgctccatacagaaaggttagaaaggttgttatggtcaactct
attccaaagtctccaactggtaagattttgagaaaggaattgaag
aggattttgactaactctgtttcttaa
SEQ ID NO: 11:
At4CLL6 (protein)
MAATHLHIPPNPKTQTSHONPPFWFSSKTGIYTSKFPSLHLPVDP
NLDAVSALFSHKHHGDTALIDSLTGFSISHTELQIMVQSMAAGIY
HVLGVRQGDVVSLVLPNSVYFPMIFLSLISLGAIVTTMNPSSSLG
EIKKQVSECSVGLAFTSTENVEKLSSLGVSVISVSESYDFDSIRI
ENPKFYSIMKESFGFVPKPLIKQDDVAAIMYSSGTTGASKGVLLT
HRNLIASMELFVRFEASQYEYPGSSNVYLAALPLCHIYGLSLFVM
GLLSLGSTIVVMKRFDASDVVNVIERFKITHFPVVPPMLMALTKK
AKGVCGEVFKSLKQVSSGAAPLSRKFIEDFLQTLPHVDLIQGYGM
TESTAVGTRGFNSEKLSRYSSVGLLAPNMQAKVVDWSSGSFLPPG
NRGELWIQGPGVMKGYLNNPKATQMSIVEDSWLRTGDIAYFDEDG
YLFIVDRIKEIIKYKGFQIAPADLEAVLVSHPLIIDAAVTAAPNE
ECGEIPVAFVVRRQETTLSEEDVISYVASQVAPYRKVRKVVMVNS
IPKSPTGKILRKELKRILTNSVS

In one embodiment, the sequence identity is at least 50%. In another embodiment, the sequence identity is at least 75%. In another embodiment, the sequence identity is at least 95%.

In another embodiment, the sequence identity is at least 99% with a protein sequence utilized herein. In another embodiment, the sequence identity is at least 99% with a nucleic acid sequence utilized herein.

In another embodiment, the heterologous microorganism is antibiotic-marker free. In another embodiment, the heterologous microorganism is an FAA2 (peroxisomal medium chain fatty acyl-CoA synthetase) knock out or has a lowered FAA2 activity. In another embodiment, the heterologous microorganism is a PXA1 (part of the heterodimeric peroxisomal fatty acid and/or acyl-CoA ABC transport complex with PXA2) knockout or has a lowered PXA2 activity. In another embodiment, the heterologous microorganism is a PEX11 (peroxisomal protein required for medium-chain fatty acid oxidation) knockout or has a lowered PEX11 activity. In another embodiment, the heterologous microorganism is an ANT1 (peroxisomal adenine nucleotide transporter, which exchanges AMP generated in peroxisomes by acyl-CoA synthetases for ATP, that is consumed in that reaction, from the cytosol) knockout or has a lowered ANT1 activity.

In another embodiment, the microorganism is Saccharomyces cerevisiae. In another embodiment, the Saccharomyces cerevisiae comprises galactose regulatable promoters for the heterologous genes (csOLS, csOAC, csAAE, and the likes). In another embodiment, the Saccharomyces cerevisiae does not include galactose regulatable promoters for the heterologous genes. In another embodiment, the Saccharomyces cerevisiae is haploid. In another embodiment, the Saccharomyces cerevisiae is diploid.

Initial construction of an olivetol/olivetolic acid (O/OA) producing line was done by introducing 3 genes (cannabis olivetol synthase csOLS, cannabis olivetolic acid cyclase csOAC, and a cannabis acyl-activating enzyme csAAE1) from the Cannabis sativa plant under the control of genetic elements that are regulated by a galactose carbon source. It is well known that galactose regulates gene expression in Saccharomyces cerevisiae. Introduction of foreign genes into Saccharomyces cerevisiae can be toxic to Saccharomyces cerevisiae for a variety of reasons. One way to regulate toxicity of foreign genes is to produce their expression under the control of galactose. Glucose is used under normal Saccharomyces cerevisiae growth conditions. However, during the course of growth or at the beginning of growth, glucose can then be exchanged with galactose to tightly control the expression of foreign genes.

The initial engineering of Saccharomyces cerevisiae was done by introducing a gene fragment containing csOLS, csOAC, and csAAE1 under the control of galactose regulatable elements called promoters. The csOLS and csOAC were physically linked to each other on the gene with a genetic element called T2A in some examples and was separated in other examples. In order to select for Saccharomyces cerevisiae cells that efficiently incorporated the foreign DNA, but removed Saccharomyces cerevisiae that had no foreign genes, a method that allows growth on nutrient preferred media was utilized. The process by which Saccharomyces cerevisiae uptake foreign DNA and stably utilize the foreign DNA is called recombination. The final Saccharomyces cerevisiae strains that took up the foreign DNA and utilized the DNA are called recombinants. Saccharomyces cerevisiae that did not undergo recombination is called wild type. The process of selecting in preferred media is defined as prototrophy rescue. In order to separate recombinants from wild type we utilized prototrophy rescue. In some cases, recombinants we added foreign genes that contained genetic elements that controlled the resistance to antibiotics. Antibiotics such as G418 or hygromycin will normally kill Saccharomyces cerevisiae. In some instances, foreign genes were introduced into O/OA producing lines in order to rescue survival of G418 or hygromycin for the purpose of removing genes native to the Saccharomyces cerevisiae and allowing them to survive in antibiotics while decreasing the need for galactose utilization.

### Expression Vectors

In various aspects, provided herein is a recombinant host cell modified by genetic engineering as disclosed herein. In one embodiment, a recombinant polyketide synthase, such as an OLS enzyme, is introduced. In another embodiment, an aromatic prenyltransferase is introduced. In another embodiment, the modification increases the production of malonyl-CoA, hexanoyl-CoA or a R-CoA. In some embodiments, the host cell is engineered via recombinant DNA technology to express heterologous nucleic acids that encode a cannabinoid pathway enzyme such as an OLS enzyme, which is either a mutated version of a naturally occurring enzyme, or a non-naturally occurring enzyme as provided herein.

In one preferred embodiment, the invention includes methods of generating a polynucleotide that expresses one or more of the SEQ IDs related to a mutant or modified OLS provided or utilized herein. In certain preferred embodiments, the proteins of the invention are expressed using any of a number of systems, such as in whole plants, as well as plant cell and/or yeast suspension cultures. E.g., the polynucleotide that encodes the OLS is placed under the control of a promoter that is functional in the desired host cell. An extremely wide variety of promoters may be available and can be used in the expression vectors of the invention, depending on the particular application. Ordinarily, the promoter selected depends on the cell in which the promoter is to be active. Other expression control sequences such as ribosome binding sites, transcription termination sites and the like are also optionally included.

Nucleic acid constructs provided and utilized herein include expression vectors that comprise nucleic acids encoding one or more polyketide synthase enzymes. The nucleic acids encoding the enzymes are operably linked to promoters and optionally other control sequences such that the subject enzymes are expressed in a host cell containing the expression vector when cultured under suitable conditions. The promoters and control sequences employed depend on the host cell selected for the production of olivetol, olivetolic acid (OLA or OA), OLA-derived compound, or another cannabinoid or cannabinoid derivative. Thus, the invention provides not only expression vectors but also nucleic acid constructs useful in the construction of expression vectors. Methods for designing and making nucleic acid constructs and expression vectors generally are well known to those skilled in the art and so are only briefly reviewed herein.

Nucleic acids encoding the polyketide synthase enzymes can be prepared by any suitable method known to those of ordinary skill in the art, including, for example, direct chemical synthesis and cloning. Further, nucleic acid sequences for use in the invention can be obtained from commercial vendors that provide de novo synthesis of the nucleic acids.

A nucleic acid encoding the desired enzyme can be incorporated into an expression vector by known methods that include, for example, the use of restriction enzymes to cleave specific sites in an expression vector, e.g., plasmid, thereby producing an expression vector of the invention. Some restriction enzymes produce single stranded ends that may be annealed to a nucleic acid sequence having, or synthesized to have, a terminus with a sequence complementary to the ends of the cleaved expression vector. The ends are then covalently linked using an appropriate enzyme, e.g., DNA ligase. DNA linkers may be used to facilitate linking of nucleic acids sequences into an expression vector.

A set of individual nucleic acid sequences can also be combined by utilizing polymerase chain reaction (PCR)-based methods known to those of skill in the art. For example, each of the desired nucleic acid sequences can be initially generated in a separate PCR. Thereafter, specific primers are designed such that the ends of the PCR products contain complementary sequences. When the PCR products are mixed, denatured, and reannealed, the strands having the matching sequences at their 3′ ends overlap and can act as primers for each other. Extension of this overlap by DNA polymerase produces a molecule in which the original sequences are “spliced” together. In this way, a series of individual nucleic acid sequences may be joined and subsequently transduced into a host cell simultaneously. Thus, expression of each of the plurality of nucleic acid sequences is affected.

A typical expression vector contains the desired nucleic acid sequence preceded and optionally followed by one or more control sequences or regulatory regions, including a promoter and, when the gene product is a protein, ribosome binding site, e.g., a nucleotide sequence that is generally 3-9 nucleotides in length and generally located 3-11 nucleotides upstream of the initiation codon that precede the coding sequence, which is followed by a transcription terminator in the case of E. coli or other prokaryotic hosts. See Shine et al., Nature 254:34 (1975) and Steitz, in Biological Regulation and Development: Gene Expression (ed. R. F. Goldberger), vol. 1, p. 349 (1979) Plenum Publishing, N.Y. In the case of eukaryotic hosts like yeast, a typical expression vector contains the desired nucleic acid coding sequence preceded by one or more regulatory regions, along with a Kozak sequence to initiate translation and followed by a terminator. See Kozak, Nature 308:241-246 (1984).

Regulatory regions or control sequences include, for example, those regions that contain a promoter and an operator. A promoter is operably linked to the desired nucleic acid coding sequence, thereby initiating transcription of the nucleic acid sequence via an RNA polymerase. An operator is a sequence of nucleic acids adjacent to the promoter, which contains a protein-binding domain where a transcription factor can bind. Transcription factors activate or repress transcription initiation from a promoter. In this way, control of transcription is accomplished, based upon the particular regulatory regions used and the presence or absence of the corresponding transcription factor. Non-limiting examples for prokaryotic expression include lactose promoters (LacI repressor protein changes conformation when contacted with lactose, thereby preventing the LacI repressor protein from binding to the operator) and tryptophan promoters (when complexed with tryptophan, TrpR repressor protein has a conformation that binds the operator; in the absence of tryptophan, the TrpR repressor protein has a conformation that does not bind to the operator). Non-limiting examples of promoters to use for eukaryotic expression include pTDH3, pTEF1, pTEF2, pRNR2, pRPL18B, pREV1, pGAL1, pGAL10, pGAPDH, pCUP1, pMET3, pPGK1, pPYK1, pHXT7, pPDC1, pFBA1, pTDH2, pPGI1, pPDC1, pTPI1, pENO2, pADH1, and pADH2. As will be appreciated by those of ordinary skill in the art, a variety of expression vectors and components thereof are useful.

Although any suitable expression vector are useful to incorporate the desired sequences, readily available expression vectors include, without limitation: plasmids, such as pESC, pTEF, p414CYC1, p414GALS, pSC101, pBR322, pBBR1MCS-3, pUR, pEX, pMR100, pCR4, pBAD24, pUC19, pRS series; and bacteriophages, such as M13 phage and λ phage. Of course, such expression vectors may only be suitable for particular host cells or for expression of particular polyketide synthases. One of ordinary skill in the art, however, can readily determine through routine experimentation whether any particular expression vector is suited for any given host cell or protein. For example, the expression vector can be introduced into the host cell, which is then monitored for viability and expression of the sequences contained in the vector. In addition, relevant texts and literature describe expression vectors and their suitability to any particular host cell. In addition to the use of expression vectors, strains are built where expression cassettes are directly integrated into the host genome.

The expression vectors are introduced or transferred, e.g., by transduction, transfection, or transformation, into the host cell. Such methods for introducing expression vectors into host cells are well known to those of ordinary skill in the art. For example, one method for transforming P. kudriavzevii with an expression vector involves a calcium chloride treatment wherein the expression vector is introduced via a calcium precipitate.

For identifying whether a nucleic acid has been successfully introduced or into a host cell, a variety of methods are available. For example, potentially transformed host cells in a culture are separated, using a suitable dilution, into individual cells and thereafter individually grown and tested for expression of a desired gene product of a gene contained in the introduced nucleic acid. For example, an often-used practice involves the selection of cells based upon antibiotic resistance that has been conferred by antibiotic resistance-conferring genes in the expression vector, such as the beta lactamase (amp), aminoglycoside phosphotransferase (neo), and hygromycin phosphotransferase (hyg, hph, hpt) genes.

In one embodiment, a host cell of the disclosure is transformed with at least one expression vector. When only a single expression vector is used, the vector will typically contain a polyketide synthase gene. Once the host cell has been transformed with the expression vector, the host cell is cultured in a suitable medium containing a carbon source, such as a sugar (e.g., glucose). As the host cell is cultured, expression of the polyketide synthase enzyme(s) occurs. Once expressed, these OLS(s) and other enzymes provided and utilized herein convert three molecules of malonyl-CoA and one molecule of hexanoyl-CoA or R-CoA, wherein R is defined as herein, to olivetol or a compound of formula (I).

If a host cell of the invention is to include more than one heterologous gene, the multiple genes can be expressed from one or more vectors. For example, a single expression vector can comprise one, two, or more genes encoding one, two, or more mutant OLS enzyme(s), other enzymes of the cannabinoid pathway, e.g., improved malonyl-CoA production, hexanoyl-CoA, or R-CoA production, etc. The heterologous genes can be contained in a vector replicated episomally or in a vector integrated into the host cell genome, and where more than one vector is employed, then all vectors may replicate episomally (extrachromasomally), or all vectors may integrate, or some may integrate and some may replicate episomally. While a “gene” is generally composed of a single promoter and a single coding sequence, in certain host cells, two or more coding sequences are controlled by one promoter in an operon. In some embodiments, a two or three operon system is used.

In some embodiments, the coding sequences employed have been modified, relative to some reference sequence, to reflect the codon preference of a selected host cell. Codon usage tables for numerous organisms are readily available and can be used to guide sequence design. The use of prevalent codons of a given host organism generally improves translation of the target sequence in the host cell. As one non-limiting example, in some embodiments the subject nucleic acid sequences will be modified for yeast codon preference (see, for example, Bennetzen et al., J. Biol. Chem. 257:3026-3031 (1982)). In some embodiments, the nucleotide sequences will be modified for P. kudriavzevii codon preference (see, for example, Nakamura et al., Nucleic Acids Res. 28:292 (2000)). In other embodiments, the nucleotide sequences are modified to include codons optimized for S. cerevisiae codon preference.

Nucleic acids can be prepared by a variety of routine recombinant techniques. Briefly, the subject nucleic acids can be prepared from genomic DNA fragments, cDNAs, and RNAs, all of which can be extracted directly from a cell or recombinantly produced by various amplification processes including but not limited to PCR and rt-PCR. Subject nucleic acids can also be prepared by a direct chemical synthesis.

The nucleic acid transcription levels in a host microorganism can be increased (or decreased) using numerous techniques. For example, the copy number of the nucleic acid can be increased through use of higher copy number expression vectors comprising the nucleic acid sequence, or through integration of multiple copies of the desired nucleic acid into the host microorganism's genome. Non-limiting examples of integrating a desired nucleic acid sequence onto the host chromosome include recA-mediated recombination, lambda phage recombinase-mediated recombination and transposon insertion. Nucleic acid transcript levels can be increased by changing the order of the coding regions on a polycistronic mRNA or breaking up a polycistronic operon into multiple poly- or mono-cistronic operons each with its own promoter. RNA levels can be increased (or decreased) by increasing (or decreasing) the strength of the promoter to which the protein-coding region is operably linked.

The translation level of a desired polypeptide sequence in a host microorganism can also be increased in a number of ways. Non-limiting examples include increasing the mRNA stability, modifying the ribosome binding site (or Kozak) sequence, modifying the distance or sequence between the ribosome binding site (or Kozak sequence) and the start codon of the nucleic acid sequence coding for the desired polypeptide, modifying the intercistronic region located 5′ to the start codon of the nucleic acid sequence coding for the desired polypeptide, stabilizing the 3′-end of the mRNA transcript, modifying the codon usage of the polypeptide, altering expression of low-use/rare codon tRNAs used in the biosynthesis of the polypeptide. Determination of preferred codons and low-use/rare codon tRNAs can be based on a sequence analysis of genes derived from the host microorganism.

The polypeptide half-life, or stability, can be increased through mutation of the nucleic acid sequence coding for the desired polypeptide, resulting in modification of the desired polypeptide sequence relative to the control polypeptide sequence. When the modified polypeptide is an enzyme, the activity of the enzyme in a host is altered due to increased solubility in the host cell, improved function at the desired pH, removal of a domain inhibiting enzyme activity, improved kinetic parameters (lower Km or higher k_{cat} values) for the desired substrate, removal of allosteric regulation by an intracellular metabolite, and the like. Altered/modified enzymes can also be isolated through random mutagenesis of an enzyme, such that the altered/modified enzyme can be expressed from an episomal vector or from a recombinant gene integrated into the genome of a host microorganism.

### Host Cells

Provided herein are host cells, preferably recombinant host cells, more preferably heterologous recombinant host cells for performing one or more steps of the cannabinoid pathway. In some embodiments, the recombinant host cell is a eukaryote. In various embodiments, the eukaryote is a yeast strain selected from the non-limiting list of example genera: Candida, Cryptococcus, Hansenula, Issatchenkia, Kluyveromyces, Komagataella, Lipomyces, Pichia, Rhodosporidium, Rhodotorula, Saccharomyces, or Yarrowia. Those skilled in the art will recognize that these genera broadly encompass yeast, including those distinguished as oleaginous yeast. In some embodiments, the host cell is Saccharomyces cerevisiae. In other embodiments, the host cell is Pichia kudriavzevii. In other embodiments of the invention, the eukaryotic host cell is a fungus or algae. In yet other embodiments, the recombinant host cell is a prokaryote selected from the non-limited example genera: Bacillus, Clostridium, Corynebacterium, Escherichia, Pseudomonas, Rhodobacter, and Streptomyces. In various embodiments, the host cell is P. kudriavzevii.

In one embodiment, the host cell is part of a multicellular organism. In one embodiment, the multicellular organism is a plant. In one embodiment, the plant is a cannabis plant. In one embodiment, the plant is a tobacco plant.

As utilized herein, a number of genetic modifications are further useful for increasing microbial biosynthesis of malonyl-CoA. For example, in some embodiments a host cell provided or utilized herein is further engineered to include a genetic modification useful for converting pyruvate to malonyl-CoA, wherein the genetic modification produces and/or provides a pyruvate decarboxylase, an acetaldehyde dehydrogenase, an acetyl-CoA synthetase, an acetyl-CoA carboxylase, and a carbonic anhydrase.

In some embodiments, an engineered host cell provided or utilized herein is a Saccharomyces cerevisiae host cell. In some embodiments, an engineered host cell comprises heterologous enzymes that are overexpressed to increase malonyl-CoA production, thereby facilitating production of olivetol, OLA, OLA-derived compound, or another cannabinoid or cannabinoid derivative. In some embodiments, the engineered host cell comprises heterologous enzymes selected from the group consisting of an acetyl Co-A carboxylase, such as P. kudriavzevii acetyl-CoA carboxylase, S. cerevisiae aldehyde dehydrogenase, Yarrowia lipolytica acetyl-CoA synthetase, and S. cerevisiae pyruvate decarboxylase.

In some embodiments, the host cell is a Saccharomyces cerevisiae host cell. In some embodiments, a yeast host cell expressing an OLS is used to produce olivetol, OLA, OLA-derived compound, or another cannabinoid or cannabinoid derivative. In some embodiments, an oleaginous yeast host cell expressing an OLS is used to produce olivetol, OLA, OLA-derived compound, or another cannabinoid or cannabinoid derivative.

Also provided herein is a mutated OLS comprising a mutated active site, vectors for expressing the mutant, and host cells that express the mutant. In another embodiment, the host cell further produces olivetol, OLA, OLA-derived compound, or another cannabinoid or cannabinoid derivative. Introduction of mutations in the region comprising D198 to G209 of OLA increases the turnover rate (i.e., k_{cat} values) of the mutated OLS. One or more point mutations at amino acid positions D198 to G209 can be introduced alone or in any desired combination. In these embodiments, the recombinant host cell can be, without limitation, a P. kudriavzevii or yeast, including but not limited to S. cerevisiae or other yeast, host cell.

In some aspects, provided herein are recombinant host cells, preferably host cells suitable for producing olivetol (including OLA and/or OLA-derived compounds) and other cannabinoids and cannabinoid derivatives in accordance with the methods provided herein, the host cells comprising one or more heterologous OLS enzymes, preferably OLS enzymes having an increased k_{cat} value as compared to wild type or homologous OLS enzymes, wherein the recombinant host cells provide increase olivetol titer, yield, and/or productivity relative to a host cell not comprising a heterologous OLS enzyme. In some aspects, provided herein are recombinant host cells suitable for producing olivetol in accordance with the methods of the invention comprising increased malonyl-CoA biosynthesis. In some aspects, provided herein are recombinant host cells suitable for producing olivetol in accordance with the methods of the invention comprising increased hexanoyl-CoA synthetase biosynthesis. In some aspects, provided herein are recombinant host cells suitable for producing olivetol in accordance with the methods of the invention comprising increased pyruvate dehydrogenase biosynthesis. In some aspects, provided herein are recombinant host cells suitable for producing olivetol in accordance with the methods of the invention comprising increased acetaldehyde dehydrogenase biosynthesis. In some aspects, provided herein are recombinant host cells suitable for producing olivetol in accordance with the methods of the invention comprising increased acetyl-CoA synthetase biosynthesis. In some aspects, provided herein are recombinant host cells suitable for producing olivetol in accordance with the methods of the invention comprising increased acetyl-CoA carboxylase biosynthesis. In some aspects, provided herein are recombinant host cells suitable for producing olivetol in accordance with the methods of the invention comprising increased carbonic anhydrase biosynthesis.

In accordance with the invention, increased olivetol titer, yield, and/or productivity can be achieved through increased OLS enzymatic activity, which may require increased malonyl-CoA biosynthesis, and the invention provides host cells, vectors, enzymes, and methods relating thereto. Malonyl-CoA is produced in host cells through the activity of an acetyl-CoA carboxylase (EC 6.4.1.2) catalyzing the formation of malonyl-CoA from acetyl-CoA and carbon dioxide. The invention provides recombinant host cells for producing olivetol that express a heterologous acetyl-CoA carboxylase (ACC). In some embodiments, the host cell is a S. cerevisiae cell comprising a heterologous S. cerevisiae acetyl-CoA carboxylase ACC1 or an enzyme homologous thereto. In some embodiments, the host cell modified for heterologous expression of an ACC such as S. cerevisiae ACC1 is further modified to eliminate ACC1 post-translational regulation by genetic modification of S. cerevisiae SNF1 protein kinase or an enzyme homologous thereto. The disclosure also provides a recombinant host cell suitable for producing olivetol in accordance with the invention that is an E. coli cell that comprises a heterologous nucleic acid coding for expression of E. coli acetyl-CoA carboxylase complex proteins AccA, AccB, AccC and AccD or one or more enzymes homologous thereto.

Thus, in one aspect of the invention, the recombinant host cell comprises a heterologous nucleic acid encoding a mutant OLS enzyme or another mutant cannabinoid pathway enzyme, that results in increased production of olivetol, OLA, OLA-derived compound, or another cannabinoid or cannabinoid derivative relative to host cells not comprising the mutant OLS enzyme and/or an OLS enzyme.

Thus, in accordance with the invention an OLS enzyme other than, or in addition to, OLS derived from C. sativa can be used for biological synthesis of olivetol, OLA, OLA-derived compound, or another cannabinoid or cannabinoid derivative in a recombinant host. In some embodiments, the recombinant host is P. kudriavzevii. In some embodiments, the recombinant host is S. cerevisiae. In other embodiments, the recombinant host is E. coli. In other embodiments, the recombinant host is a yeast other than P. kudriavzevii. In various embodiments, the host is modified to express a mutated OLS enzyme and/or an OLS enzyme provided or utilized herein. In various embodiments, the host is further modified to express one or more heterologous enzymes that are overexpressed to increase malonyl-CoA production. In various embodiments, the host is further modified to express or overexpress a functional hexanoyl-CoA synthetase.

Moreover, additional enzymes and catalysts other than those specifically disclosed herein can be utilized in mutated or heterologously expressed form. It will be well understood to those skilled in the art in view of this disclosure how other appropriate enzymes can be identified, modified, and expressed to achieve the desired olivetol, OLA, OLA-derived compound, or another cannabinoid or cannabinoid derivative production, as disclosed herein.

In one aspect, provided herein are recombinant host cells suitable for biological production of cannabinoids and derivatives, such as without limitation olivetol, OLA, OLA-derived compound, or another cannabinoid or cannabinoid derivative. Any suitable host cell is useful in practice of the methods provided herein. In some embodiments, the host cell is a recombinant host microorganism in which nucleic acid molecules have been inserted, deleted or modified (i.e., mutated; e.g., by insertion, deletion, substitution, and/or inversion of nucleotides), either to produce olivetol, or to increase yield, titer, and/or productivity of olivetol relative to a “wild type”, “control cell”, “parental cell”, or “reference cell”. A “control cell” can be used for comparative purposes, and is typically a wild type or recombinant parental cell that does not contain one or more of the modification(s) made to the host cell of interest.

In some embodiments, the invention provides a recombinant host cell that has been modified to produce one or more enzymes that facilitate malonyl-CoA production. In some embodiments, the invention provides a recombinant host cell that has been modified to produce one or more enzymes of the cannabinoid pathway. In some embodiments, the invention provides a recombinant host cell that has been modified to produce an OLS, such as, without limitation, an engineered or modified OLS, for example, olivetol synthase, having improved k_{cat} values. In some embodiments, the invention provides a recombinant host cell that has been modified to produce an OLS, such as, without limitation, an engineered or modified OLS having improved solubility in the host. In some embodiments, the invention provides a recombinant host cell that has been modified to produce an OLS, such as, without limitation, an engineered or modified OLS or having improved stability in the host. Thus, various embodiments of the invention provide recombinant host cells capable of producing increased amounts of olivetol, OLA, OLA-derived compound, or another cannabinoid or cannabinoid derivative (i.e., product) per unit time. Accordingly, various embodiments of the invention provide recombinant host cells capable of achieving higher titers of product over shorter fermentation run times.

With respect to production titer levels, the recombinant host cells provided or utilized herein produce titer levels that exceed production titer levels of control cells. In some embodiments, the recombinant host cells provided or utilized herein produce titer levels that are suitable for commercial production, for example approximately 1-20 g/L, such as 2-10 g/L or 3-8 g/L, or greater. The recombinant host cells described herein promote high titer levels of product(s) in at least two ways. First, the recombinant host cells produce mutated OLS enzymes having improved synthetase kinetics (i.e., an increase in k_{cat}), which allows for faster product production, thereby increasing the rate and ease at which a desired titer level can be achieved. Secondly, the materials and methods provided or utilized herein provide and facilitate in situ extraction of the product into an organic phase, as described below. By adding the organic phase directly to the broth during the fermentation, the product can be quickly and continuously separated from the fermentation process, thereby decreasing undesirable effects of the product on the fermentation process, such as toxicity and product inhibition feedback on the pathway enzymes, thereby further increasing the titer levels of the product(s). Additionally, various genetic modifications provided or utilized herein are useful for increasing the provision of malonyl-CoA, which is a substrate for OLS.

In one embodiment, provided herein are recombinant yeast cells suitable for the production of cannabinoids and derivatives such as, without limitation, olivetol, at levels sufficient for subsequent purification and use as described herein. Yeast host cells are excellent host cells for construction of recombinant metabolic pathways comprising heterologous enzymes catalyzing production of small molecule products. There are established molecular biology techniques and nucleic acids encoding genetic elements necessary for construction of yeast expression vectors, including, but not limited to, promoters, origins of replication, antibiotic resistance markers, auxotrophic markers, terminators, and the like. Second, techniques for integration of nucleic acids into the yeast chromosome are well established. Yeast also offers a number of advantages as an industrial fermentation host. Yeast can tolerate high concentrations of organic acids and maintain cell viability at low pH and can grow under both aerobic and anaerobic culture conditions, and there are established fermentation broths and fermentation protocols. The ability of a strain to propagate and/or produce desired product under low pH provides a number of advantages. First, this characteristic provides tolerance to the environment created by the production of an alkyl resorcinol, or an alkyl resorcinol carboxylic acid. Second, from a process standpoint, the ability to maintain a low pH environment limits the number of organisms that are able to contaminate and spoil a batch.

In some embodiments of the invention, the recombinant host cell comprising a heterologous nucleic acid provided or utilized herein is a eukaryote. In various embodiments, the eukaryote is a yeast selected from the non-limiting list of genera; Saccharomyces, Candida, Cryptococcus, Hansenula, Issatchenki, Kluyveromyces, Komagataella, Lipomyces, Pichia, Rhodosporidium, Rhodotorula, or Yarrowia species. In various embodiments, the yeast is of a species selected from the group consisting of Candida albicans, Candida ethanolica, Candida krusei, Candida methanosorbosa, Candida sonorensis, Candida tropicalis, Cryptococcus curvatus, Hansenula polymorpha, Issatchenkia orientalis, Kluyveromyces lactis, Kluyveromyces marxianus, Kluyveromyces thermotolerans, Komagataella pastoris, Lipomyces starkeyi, Pichia angusta, Pichia deserticola, Pichia galeiformis, Pichia kodamae, Pichia kudriavzevii, Pichia membranaefaciens, Pichia methanolica, Pichia pastoris, Pichia salictaria, Pichia stipitis, Pichia thermotolerans, Pichia trehalophila, Rhodosporidium toruloides, Rhodotorula glutinis, Rhodotorula graminis, Saccharomyces bayanus, Saccharomyces boulardi, Saccharomyces cerevisiae, Saccharomyces kluyveri, and Yarrowia lipolytica. One skilled in the art will recognize that this list encompasses yeast in the broadest sense, including both oleaginous and non-oleaginous strains.

Other recombinant host cells provided or utilized herein include without limitation, eukaryotic, prokaryotic, and archaea cells. Illustrative examples of eukaryotic cells include, but are not limited to: Aspergillus niger, Aspergillus oryzae, Crypthecodinium cohnii, Cunninghamella japonica, Entomophthora coronata, Mortierella alpina, Mucor circinelloides, Neurospora crassa, Pythium ultimum, Schizochytrium limacinum, Thraustochytrium aureum, Trichoderma reesei and Xanthophyllomyces dendrorhous. In general, if a eukaryotic cell is used, a non-pathogenic strain is employed. Illustrative examples of non-pathogenic strains include but are not limited to: Pichia pastoris and Saccharomyces cerevisiae. In addition, certain strains, including Saccharomyces cerevisiae, have been designated by the Food and Drug Administration as Generally Regarded As Safe (or GRAS) and so can be conveniently employed in various embodiments of the methods of the invention.

Illustrative and non-limiting examples of recombinant prokaryotic host cells provided or utilized herein include, Bacillus subtilis, Brevibacterium ammoniagenes, Clostridium beigerinckii, Corynebacterium glutamicum, Escherichia coli, Enterobacter sakazakii, Lactobacillus acidophilus, Lactococcus lactis, Mesorhizobium loti, Pseudomonas aeruginosa, Pseudomonas putida, Rhodobacter capsulatus, Rhodobacter sphaeroides, Salmonella enterica, Salmonella typhi, Salmonella typhimurium, Shigella flexneri, Staphylococcus aureus, Streptomyces ambofaciens, Streptomyces aureofaciens, Streptomyces aureus, Streptomyces fungicidicus, Streptomyces griseochromogenes, Streptomyces griseus, Streptomyces lividans, Streptomyces olivogriseus, Streptomyces rameus, Streptomyces tanashiensis, and Streptomyces vinaceus. Certain of these cells, including Bacillus subtilis, Corynebacterium glutamicum, and Lactobacillus acidophilus, have been designated by the Food and Drug Administration as Generally Regarded As Safe (or GRAS) and so are employed in various embodiments of the methods of the invention. While desirable from public safety and regulatory standpoints, GRAS status does not impact the ability of a host strain to be used in the practice of this invention; hence, non-GRAS and even pathogenic organisms are included in the list of illustrative host strains suitable for use in the practice of this invention.

Escherichia coli and Corynebacterium glutamicum are suitable prokaryotic host cells for metabolic pathway construction. Wild type E. coli can catabolize both pentose and hexose sugars as carbon sources. Provided herein are variety of E. coli host cells suitable for the production of malonate as described herein. In various embodiments, the recombinant host cell comprising a heterologous nucleic acid provided or utilized herein is an E. coli cell. In various embodiments of the methods of the invention, the recombinant host cell comprising a heterologous nucleic acid provided or utilized herein is a C. glutamicum cell.

### Fermentation

In one embodiment, the fermentation is performed at a pH of about 5-6, preferably at about 5.5. In one embodiment, the fermentation is performed at a temperature of about 30° C. In one embodiment, the organic solvent immiscible with aqueous phase employed in the fermentation is loaded at about 26% of total fermentation tank volume. In one embodiment, the organic solvent immiscible with aqueous phase employed in the fermentation is loaded at about 40% of initial fermentation tank volume. In one embodiment, Isopropyl myristate is the aqueous phase immiscible organic solvent. In one embodiment, the aqueous phase immiscible organic solvent is added about 12 to about 36 hours post inoculation.

In one embodiment, the fermentation is performed wherein the compound of formula RCO₂H or a salt thereof is present in an amount of 0.1-0.3 moles/500 g of glucose in feed. In one embodiment, the fermentation is performed wherein the compound of formula RCO₂H or a salt thereof is present in an amount of 0.14-0.25 moles/500 g of glucose in feed. In one embodiment, the fermentation is performed wherein the compound of formula RCO₂H or a salt thereof is present in an amount of 0.14-0.21 moles/500 g of glucose in feed. In one embodiment, the fermentation is performed wherein the sodium hexanoate or another hexanoate salt/glucose in feed ratio was in the range of about 20 to about 28 g sodium hexanoate or a molar equivalent thereof/500 g glucose. In one embodiment, the fermentation is performed wherein the sodium hexanoate/glucose in feed ratio was in the range of about 23 to about 28 g sodium hexanoate or a molar equivalent thereof/500 g glucose.

In one embodiment, the fermentation is performed wherein the sodium butyrate or another butyrate salt/glucose in feed ratio was in the range of about 8 to about 28 g or a molar equivalent of sodium butyrate/500 g glucose. In one embodiment, the fermentation is performed wherein the sodium butyrate/glucose in feed ratio was in the range of about 10 to about 24 g or a molar equivalent of sodium butyrate/500 g glucose. In one embodiment, the fermentation is performed wherein the sodium butyrate/glucose in feed ratio was in the range of about 10 to about 14 g or a molar equivalent of sodium butyrate/500 g glucose.

In one embodiment, the oxygen transmission rate (OTR) is about 60-about 80 mmoles/L/hr. In one embodiment, an oxygen uptake rate (OUR) of about 100-about 110 mmoles/L/hr is achieved. In one embodiment, the pulse parameter was about 1.7 g glucose/L initial tank volume/pulse with a feed rate of about 10 g/L of initial tank volume/hr. In one embodiment, the batch glucose concentration employed in the fermentation was about 10-about 20 g/L.

### Synthesis, Utilization, and Purification of Cannabinoids and Derivatives

In some aspects, provided herein are methods of producing a cannabinoid, a cannabinoid derivative, a cannabinoid precursor, or a cannabinoid precursor derivative. In some embodiments, the methods may involve culturing a genetically modified host cell of the present disclosure in a suitable medium and recovering the produced cannabinoid, the cannabinoid precursor, the cannabinoid precursor derivative, or the cannabinoid derivative. The methods may also involve cell-free production of cannabinoids, cannabinoid precursors, cannabinoid precursor derivatives, or cannabinoid derivatives using one or more polypeptides disclosed herein expressed or overexpressed by a genetically modified host cell of the disclosure.

In some embodiments, provided herein are methods of producing a cannabinoid or a cannabinoid derivative. The methods may involve culturing a genetically modified host cell of the present disclosure in a suitable medium and recovering the produced cannabinoid or cannabinoid derivative. The methods may also involve cell-free production of cannabinoids or cannabinoid derivatives using one or more polypeptides disclosed herein expressed or overexpressed by a genetically modified host cell of the disclosure.

Cannabinoids, cannabinoid derivatives, cannabinoid precursors, or cannabinoid precursor derivatives that can be produced according to the present disclosure may include, but are not limited to, cannabichromene (CBC) type (e.g., cannabichromenic acid), cannabigerol (CBG) type (e.g., cannabigerolic acid), cannabidiol (CBD) type (e.g., cannabidiolic acid), Δ⁹-trans-tetrahydrocannabinol (Δ⁹-THC) type (e.g., Δ⁹-tetrahydrocannabinolic acid), Δ⁸-trans-tetrahydrocannabinol (Δ⁸-THC) type, cannabicyclol (CBL) type, cannabielsoin (CBE) type, cannabinol (CBN) type, cannabinodiol (CBND) type, cannabitriol (CBT) type, olivetolic acid, GPP, derivatives of any of the foregoing, and others as listed in Elsohly M. A. and Slade D., Life Sci.2005 Dec. 22; 78 (5): 539-48. Epub 2005 Sep. 30.

Cannabinoids or cannabinoid derivatives that can be produced with the methods or genetically modified host cells of the present disclosure may also include, but are not limited to, cannabigerolic acid (CBGA), cannabigerolic acid monomethylether, (CBGAM), cannabigerol (CBG), cannabigerol monomethylether (CBGM), cannabigerovarinic acid (CBGVA), cannabigerovarin (CBGV), cannabichromenic acid (CBCA), cannabichromene (CBC), cannabichromevarinic acid (CBCVA), cannabichromevarin (CBCV), cannabidiolic acid (CBDA), cannabidiol (CBD), cannabidiol monomethylether (CBDM), cannabidiol-C₄ (CBD-C₄), cannabidivarinic acid (CBDVA), cannabidivarin (CBDV), cannabidiorcol (CBD-C₁), Δ⁹-tetrahydrocannabinolic acid A (THCA-A), Δ⁹-tetrahydrocannabinolic acid B (THCA-B), Δ⁹-tetrahydrocannabinol (THC), Δ⁹-tetrahydrocannabinolic acid-C₄ (THCA-C₄), Δ⁹-tetrahydrocannabinol-C₄ (THC-C₄), Δ⁹-tetrahydrocannabivarinic acid (THCVA), Δ⁹-tetrahydrocannabivarin (THCV), Δ⁹-tetrahydrocannabiorcolic acid (THCA-C₁), Δ⁹-tetrahydrocannabiorcol (THC-C₁), Δ⁷-cis-iso-tetrahydrocannabivarin, Δ⁸-tetrahydrocannabinolic acid (Δ⁸-THCA), Δ⁸-tetrahydrocannabinol (Δ⁸-THC), cannabicyclolic acid (CBLA), cannabicyclol (CBL), cannabicyclovarin (CBLV), cannabielsoic acid A (CBEA-A), cannabielsoic acid B (CBEA-B), cannabielsoin (CBE), cannabielsoinic acid, cannabicitranic acid, cannabinolic acid (CBNA), cannabinol (CBN), cannabinol methylether (CBNM), cannabinol-C₄, (CBN-C₄), cannabivarin (CBV), cannabinol-C₂ (CNB-C₂), cannabiorcol (CBN-C₁), cannabinodiol (CBND), cannabinodivarin (CBVD), cannabitriol (CBT), 10-ethyoxy-9-hydroxy-delta-6a-tetrahydrocannabinol, 8,9-dihydroxyl-delta-6a-tetrahydrocannabinol, cannabitriolvarin (CBTVE), dehydrocannabifuran (DCBF), cannabifuran (CBF), cannabichromanon (CBCN), cannabicitran (CBT), 10-oxo-delta-6a-tetrahydrocannabinol (OTHC), delta-9-cis-tetrahydrocannabinol (cis-THC), 3,4,5,6-tetrahydro-7-hydroxy-alpha-alpha-2-trimethyl-9-n-propyl-2,6-methano-2H-1-benzoxocin-5-methanol (OH-iso-HHCV), cannabiripsol (CBR), trihydroxy-delta-9-tetrahydrocannabinol (triOH-THC), and derivatives of any of the foregoing.

Additional cannabinoid derivatives that can be produced with the methods or genetically modified host cells of the present disclosure may also include, but are not limited to, 2-geranyl-5-pentyl-resorcylic acid, 2-geranyl-5-(4-pentynyl)-resorcylic acid, 2-geranyl-5-(trans-2-pentenyl)-resorcylic acid, 2-geranyl-5-(4-methylhexyl)-resorcylic acid, 2-geranyl-5-(5-hexynyl) resorcylic acid, 2-geranyl-5-(trans-2-hexenyl)-resorcylic acid, 2-geranyl-5-(5-hexenyl)-resorcylic acid, 2-geranyl-5-heptyl-resorcylic acid, 2-geranyl-5-(6-heptynoic)-resorcylic acid, 2-geranyl-5-octyl-resorcylic acid, 2-geranyl-5-(trans-2-octenyl)-resorcylic acid, 2-geranyl-5-nonyl-resorcylic acid, 2-geranyl-5-(trans-2-nonenyl) resorcylic acid, 2-geranyl-5-decyl-resorcylic acid, 2-geranyl-5-(4-phenylbutyl)-resorcylic acid, 2-geranyl-5-(5-phenylpentyl)-resorcylic acid, 2-geranyl-5-(6-phenylhexyl)-resorcylic acid, 2-geranyl-5-(7-phenylheptyl)-resorcylic acid, (6aR,10aR)-1-hydroxy-6,6,9-trimethyl-3-propyl-6a, 7,8,10a-tetrahydro-6H-dibenzo[b,d]pyran-2-carboxylic acid, (6aR,10aR)-1-hydroxy-6,6,9-trimethyl-3-(4-methylhexyl)-6a,7,8,10a-tetrahydro-6H-dibenzo[b,d]pyran-2-carboxylic acid, (6aR,10aR)-1-hydroxy-6,6,9-trimethyl-3-(5-hexenyl)-6a,7,8,10a-tetrahydro-6H-dibenzo[b,d]pyran-2-carboxylic acid, (6aR,10aR)-1-hydroxy-6,6,9-trimethyl-3-(5-hexenyl)-6a,7,8,10a-tetrahydro-6H-dibenzo[b,d]pyran-2-carboxylic acid, (6aR,10aR)-1-hydroxy-6,6,9-trimethyl-3-(6-heptynyl)-6a,7,8,10a-tetrahydro-6H-dibenzo[b,d]pyran-2-carboxylic acid, 3-[(2E)-3,7-dimethylocta-2,6-dien-1-yl]-6-(hexan-2-yl)-2,4-dihydroxybenzoic acid, 3-[(2E)-3,7-dimethylocta-2,6-dien-1-yl]-2,4-dihydroxy-6-(2-methylpentyl) benzoic acid, 3-[(2E)-3,7-dimethylocta-2,6-dien-1-yl]-2,4-dihydroxy-6-(3-methylpentyl) benzoic acid, 3-[(2E)-3,7-dimethylocta-2,6-dien-1-yl]-2,4-dihydroxy-6-(4-methylpentyl) benzoic acid, 3-[(2E)-3,7-dimethylocta-2,6-dien-1-yl]-2,4-dihydroxy-6-[(1E)-pent-1-en-1-yl]benzoic acid, 3-[(2E)-3,7-dimethylocta-2,6-dien-1-yl]-2,4-dihydroxy-6-[(2E)-pent-2-en-1-yl]benzoic acid, 3-[(2E)-3,7-dimethylocta-2,6-dien-1-yl]-2,4-dihydroxy-6-[(2E)-pent-3-en-1-yl]benzoic acid, 3-[(2E)-3,7-dimethylocta-2,6-dien-1-yl]-2,4-dihydroxy-6-(pent-4-en-1-yl) benzoic acid, 3-[(2E)-3,7-dimethylocta-2,6-dien-1-yl]-2,4-dihydroxy-6-propylbenzoic acid, 3-[(2E)-3,7-dimethylocta-2,6-dien-1-yl]-2,4-dihydroxy-6-butylbenzoic acid, 3-[(2E)-3,7-dimethylocta-2,6-dien-1-yl]-2,4-dihydroxy-6-hexylbenzoic acid, 3-[(2E)-3,7-dimethylocta-2,6-dien-1-yl]-2,4-dihydroxy-6-heptylbenzoic acid, 3-[(2E)-3,7-dimethylocta-2,6-dien-1-yl]-2,4-dihydroxy-6-octylbenzoic acid, 3-[(2E)-3,7-dimethylocta-2,6-dien-1-yl]-2,4-dihydroxy-6-nonanylbenzoic acid, 3-[(2E)-3,7-dimethylocta-2,6-dien-1-yl]-2,4-dihydroxy-6-decanylbenzoic acid, 3-[(2E)-3,7-dimethylocta-2,6-dien-1-yl]-2,4-dihydroxy-6-undecanylbenzoic acid, 6-(4-chlorobutyl)-3-[(2E)-3,7-dimethylocta-2,6-dien-1-yl]-2,4-dihydroxybenzoic acid, 3-[(2E)-3,7-dimethylocta-2,6-dien-1-yl]-2,4-dihydroxy-6-[4-(methylsulfanyl) butyl]benzoic acid, and others as listed in Bow, E. W. and Rimoldi, J. M., “The Structure-Function Relationships of Classical Cannabinoids: CB1/CB2 Modulation,” Perspectives in Medicinal Chemistry 2016:8, 17-39 doi: 10.4137/PMC.S32171, incorporated herein by reference. Methods of determining the activity and properties of cannabinoids and cannabinoid derivatives are well known (see, e.g., Bow and Rimoldi, supra), and can be adapted in view of the present disclosure by the skilled artisan.

Certain non-limiting processes for producing certain compounds of formulas IA and IB are exemplified. A skilled artisan will be able to prepare other compounds of formulas IA and IB based on the disclosure provided herein and based on disclosure provided in PCT Application No.: PCT/US2021/030452, entitled “Large Scale Production Of Olivetol, Olivetolic Acid And Other Alkyl Resorcinols By Fermentation,” which is incorporated herein in its entirety by reference.

### Examples

These examples illustrate but do not limit the disclosed invention. Methods and strains useful in accordance with this invention can be adapted by a skilled artisan from U.S. Pat. No. 10,392,635 (incorporated herein by reference).

### Strain Construction Examples

Certain strains may be renumbered over time for convenience, as will be apparent to the skilled artisan.

### Example 1A: Construction of LSC3-16 and LSC3-2

LSC3-2 was iteratively constructed by transforming chemically competent JK9-3d (LSC3-1) with pAG304Gal110OSOACCSAAE1, pAG305Gal110OSOACCSAAE1 and pAG306Gal110OSOACCSAAE1 and controlling their genomic copy number at specific genomic loci. pAG304Gal110OSOACCSAAE1 and pAG306Gal110OSOACCSAAE1 were constructed by first amplifying the yeast shuttle vectors designated as pAG304 and pAG306 with the primers pAG304_fwd and pAG304_rev to amplify the Saccharomyces cerevisiae prototrophy genetic elements in addition, E. coli origins of replication and an ampicillin resistant expression cassette. The dual promoter system pGal1 and pGal10 was amplified from genomic DNA of JK9-3d with primers Gal1_10_fwd and Gal1_10_rev. The csAAE1 and OS-T2A-OAC fragments were amplified from sequences that were stored in pUC19 subcloning vectors. Amplified DNA fragments were mixed at equimolar concentrations with their respective shuttle vector sequences (pAG304 or pAG306) and preassembled by Gibson Assembly using the NEBuilder HiFi DNA Assembly Mix (NEB E5520S). The final sequences pAG304Gal110OSOACCSAAE1 and pAG306Gal110OSOACCSAAE1. To generate the DNA fragment pAG305Gal110OSOACCSAAE1, the template Gal110CBGA was amplified with Gal1_10_fwd and Gal1_10_rev to generate the yeast shuttle vector containing leucine prototrophy, dual expression promoter pGal1 and pGal10, and the OS-T2A-OAC fragment. The csAAE1 fragment was amplified from a pUC19 subcloning containing the csAAE1 gene fragment using primers CB_CSAAE1_fwd and CB_CSAAE1_rev. The amplified sequences were mixed at equimolar concentrations and assembled by Gibson Assembly using the NEBuilder HiFi DNA Assembly Mix.

First a parental strain to LSC3-2, designated as LSC3-16, was generated. LSC3-16 was generated by transforming 2 microgram (ug) of Aflll (NEB R0520S) linearized pAG305Gal110OSOACCSAAE1 into chemically competent JK9-3d mating type alpha cells that are auxotrophic to leucine, histidine, tryptophan, and uracil. Selection for pAG305Gal110OSOACCSAAE1 integration was done with leucine prototrophy rescue on yeast nitrogen base agar plates with dropout amino acid mixes deficient in leucine supplemented with 100 mg/L glucose. Genetic copy number of pAG305Gal110OSOACCSAAE1 integrated at chromosome Ill was initially quantitated by qPCR through isolation of sister clones from the transformation. The highest copy integrant was taken and designated as LSC3-16.

Chemically competent LSC3-16 were co-transformed with both pAG304Gal110OSOACCSAAE1 and pAG306Gal110OSOACCSAAE1 and selected on yeast nitrogen base agar plates with dropout amino acid mixes deficient in leucine, uracil and tryptophan supplemented with 100 mg/L glucose. Genetic copy number for pAG304Gal110OSOACCSAAE1, pAG305Gal110OSOACCSAAE1 and pAG306Gal110OSOACCSAAE1 integrated at chromosomes 4, 3 and 5 were quantitated by qPCR through isolation of genomic DNA of sister clones on selection plates. A correlation of both total polyketide and OA: O molar ratio was observed. Whole genomic sequencing was done on LSC3-16 and LSC3-2 which had achieved titers of 150 mg/L and 350 mg/L in shake flask experiments, respectively. Genetic copy numbers were determined to be 6 (LSC3-16) and 16 (LSC3-2).

LSC3-4 was generated from LSC3-2 by integrating a cassette containing PkHIS4 (HIS4 from Pichia kudriavzevii) preceded by the TEF1 promoter from Saccharomyces cerevisiae (pScTEF1) into the GAL80 locus using PCR fragments amplified from Pichia kudriavzevii genomic DNA using primers YO11334 and YO11335, and from S. cerevisiae genomic DNA using primers YO11307 and YO11308. The two PCR fragments were transformed into chemically competent LSC3-2, and colonies were selected on defined media containing Complete Supplement Mixture (CSM; Formedium, Hunstanton, UK) without histidine (CSM-His). Integration of the cassette at the GAL80 locus was confirmed by colony PCR.

### Example IC: LSC3-13 Strain

LSC3-13 was generated from LSC3-4 by integrating a cassette (HygR) containing the hph gene encoding hygromycin-B 4-O-kinase from Escherichia coli (with a GGT codon inserted immediately after the start codon) flanked by the 379 bp TEF1 promoter and 240 bp TEF1 terminator from Ashbya gossypii, into the MIG1 locus. A PCR fragment containing the HygR cassette was amplified from an in-house plasmid (pLYG-001) using primers YO11337 and YO11338. The PCR fragment was transformed into chemically competent LSC3-4, and colonies were selected on YPD medium containing 300 μg/mL hygromycin B. Integration of the cassette at the MIG1 locus was confirmed by colony PCR.

### Example ID: LSC3-18 Strain

LSC3-18 was generated from LSC3-4 by integrating the HygR cassette into the GAL1 locus. A PCR fragment containing the HygR cassette was amplified from an in-house plasmid using primers YO11436 and YO11437. The PCR fragment was transformed into chemically competent LSC3-4, and colonies were selected on YPD medium containing 300 μg/ml hygromycin. Integration of the cassette at the GAL1 locus was confirmed by colony PCR.

### Example IE: LSC3-13 Gal1\hat{ }Strain (LSC3-64, LSC3-65)

LSC3-64 and LSC3-65 were generated by transforming two PCR fragments into chemically competent LSC3-13 that together compose a split KanMX marker (with a TEF1 promoter and terminator from Ashbya gossypii flanking a kanR gene encoding an aminoglycoside phosphotransferase conferring G418 resistance) flanked by lox66 and lox71 recombination sites, replacing the GAL1 gene region. This cassette is hereafter referred to as a loxKanMX cassette. The first fragment was amplified from an internal plasmid containing the assembled KanMX cassette flanked by lox sites (pLOA-058) using primers YO11791 and YO316, binding internally in the KanMX cassette. The second PCR fragment, was generated by PCR from the same internal plasmid template using primers YO949, binding internally in the KanMX cassette, and YO11792. Colonies were selected on YPD agar plates containing 200 μg/mL G418, and two isolates (LSC3-64 and LSC3-65) containing the full length integration at the desired locus were confirmed by colony PCR.

### Example 1F: Additional Strain Construction Examples

An antibiotic marker-free version of LSC3-13 (LSC3-133 and LSC3-134) was generated by first integrating a cassette containing the HIS4 gene from S. cerevisiae CEN.PK2-1C MATa, together with its native upstream promoter and downstream terminator regions, and flanked by lox66 and lox71 recombination sites, into the GAL80 locus. This “loxHIS4” cassette was amplified in two fragments from an in-house constructed vector (pLOA-027) using primers YO11438 and YO11431, and YO11432 and YO11439. The PCR fragments were transformed into chemically competent LSC3-2, and colonies were selected on CSM-His agar plates. Integration of the cassette at the GAL80 locus was confirmed by colony PCR, and the strain was designated LSC3-46. Subsequently, the integrated functional HIS4 marker was looped out by transforming an in-house vector (pLYG-005) expressing Cre recombinase and harboring the CEN/ARS origin of replication. Transformants were selected on YPD plates containing 200 μg/mL G418 and up to 50 colonies were restruck on both G418 and YPD plates to screen for colonies that were spontaneously cured of pLYG-005 (grow on YPD but not on YPD plus G418). Cured isolates were then confirmed for loss of HIS4 by colony PCR and checking for lack of growth on CSM-His plates. One confirmed isolate was designated LSC3-103. Following this, a cassette consisting of the PkHIS4 marker with promoter and terminator as previously described, flanked with lox66 and lox71 recombination sites (hereafter referred to as a loxPkHIS4 cassette), was amplified from an in-house vector (pLOA-093) in two PCR fragments and integrated into the MIG1 locus of LSC3-103. The first fragment was amplified using primers YO12096 and YO12098, and the second fragment was amplified using primers YO12018 and YO12097. Both fragments were transformed into the chemically competent loopout strain and colonies were selected on CSM-His agar plates. Integration of the cassette into the MIG1 locus was confirmed by colony PCR.

An alternative marker-free version of LSC3-13 (LSC3-133A and LSC3-134A) was generated by integrating a cassette containing the HygR cassette previously described, flanked by the lox66 and lox71 recombination sites (hereafter referred to as a loxHygR cassette). Two PCR fragments were amplified from an in-house vector containing the loxHygR cassette (pLOA-094) using primers YO12096 and YO343, and YO189 and YO12097. The two PCR fragments were transformed into chemically competent LSC3-4 and colonies selected on YPD medium containing 300 μg/mL hygromycin B. Integration of the cassette at the MIG1 locus was confirmed by colony PCR. Subsequently, the HygR cassette was looped out by transforming the resulting strain above with pLYG-005, expressing Cre recombinase and harboring the CEN/ARS origin of replication. Transformants were selected on YPD plates containing 200 μg/mL G418 and up to 50 colonies were restruck on both YPD plus G418 and YPD plates to screen for colonies that were spontaneously cured of pLYG-005. Cured isolates were confirmed for loss of HygR by colony PCR and checking for lack of growth on YPD plus 300 μg/mL hygromycin-B plates.

LSC3-89 and LSC3-90 were generated by transforming two PCR fragments into chemically competent LSC3-13 that together comprise a split loxKanMX cassette as described above into the GPD1 locus. The first fragment was amplified as described above using primers YO11970 and YO316, and the second PCR fragment was amplified as described above using primers YO949 and YO11971. Similarly, LSC3-91 and LSC3-92 were generated in an identical way except into chemically competent LSC3-18. Colonies were selected on YPD medium containing 200 μg/mL G418 and integration in the GPD1 locus was confirmed by colony PCR.

To prevent degradation of hexanoic acid and hexanoyl-CoA through native peroxisomal β-oxidation pathways, genes were individually disrupted and tested in the LSC3-2 background. These included FAA2 (peroxisomal medium chain fatty acyl-CoA synthetase), PXA1 (part of the heterodimeric peroxisomal fatty acid and/or acyl-CoA ABC transport complex with PXA2), PEX11 (peroxisomal protein required for medium-chain fatty acid oxidation), and ANT1 (peroxisomal adenine nucleotide transporter, which exchanges AMP generated in peroxisomes by acyl-CoA synthetases for ATP, that is consumed in that reaction, from the cytosol). LSC3-48 and LSC3-49 (FAA2 knockouts) were generated by integrating a loxHIS4 cassette as 2 PCR fragments in the 3′ portion (starting at nucleotide position 412) of the FAA2 locus. The immediate 5′ portion of the gene containing the first 411 nucleotides of FAA2 and its upstream region were preserved due to overlap with the BUD25 locus transcribed from the complement strand. The two PCR fragments were amplified from pLOA-027 using primers YO11478 and YO11431, and YO11432 and YO11479, transformed into chemically competent LSC3-2, and colonies were selected CSM-His agar plates. LSC3-63 (PXA1 knockout) was generated by integrating a loxHIS4 cassette as 2 PCR fragments in the PXA1 locus. The two PCR fragments were amplified from pLOA-027 using primers YO11795 and YO11431, and YO11432 and YO11796, transformed into chemically competent LSC3-2, and colonies were selected on CSM-His agar plates. To generate the PEX11 and ANT1 knockouts, the native non-functional HIS4 locus was first knocked out in LSC3-2 by integrating a HygR cassette, generating strain LSC3-52. A PCR fragment was amplified from pLYG-001 using primers YO11709 and YO11710, transformed into chemically competent LSC3-2, and colonies were selected on YPD plus 300 μg/mL hygromycin B, with integration at the HIS4 locus confirmed by colony PCR. This strain exhibited enhanced efficiency of desired integrations using the loxHIS4 cassette, due to reduced homology with the native HIS4 locus. LSC3-74 and LSC3-75 (PEX11 knockouts) were subsequently generated by integrating a loxHIS4 cassette as 2 PCR fragments into the PEX11 locus of LSC3-52. The two PCR fragments were amplified from pLOA-027 using primers YO11498 and YO11431, and YO11432 and YO11499, transformed into chemically competent LSC3-52, and colonies were selected on CSM-His agar plates. LSC3-76 and LSC3-77 (ANT1 knockouts) were generated by integrating a loxHIS4 cassette as 2 PCR fragments into the ANT1 locus of LSC3-52. The two PCR fragments were amplified from pLOA-027 using primers YO11680 and YO11431, and YO11432 and YO11681, transformed into chemically competent LSC3-52, and colonies were selected on CSM-His agar plates. Integrations of cassettes into the desired loci were all confirmed by colony PCR for all strains.

To reduce proteolysis of the heterologously expressed pathway proteins, common proteases were additionally deleted in the LSC3-2 background. LSC3-47 (harboring a knockout of PRB1, encoding vacuolar proteinase B) was generated by integrating a loxHIS4 cassette in the PRB1 locus using 2 PCR fragments. The two PCR fragments were amplified from pLOA-027 using primers YO11488 and YO11431, and YO11432 and YO11489, transformed into chemically competent LSC3-2, and colonies were selected on CSM-His agar plates. LSC3-87 and LSC3-88 (harboring knockouts of PEP4, encoding vacuolar aspartyl protease/proteinase A) were generated by integrating a loxHIS4 cassette at the PEP4 locus in LSC3-52 using two PCR fragments. The two PCR fragments were amplified from pLOA-027 using primers YO11687 and YO11431, and YO11432 and YO11688, transformed into chemically competent LSC3-52, and colonies were selected on CSM-His agar plates. Integrations of cassettes into both desired loci were confirmed by colony PCR.

Additional knockouts can subsequently be combined from any combination of integrated lox66/lox71 flanked cassettes by transforming into strains where the previous marker was looped out by transforming pLYG-005, isolating colonies spontaneously cured for pLYG-005 with confirmed loopout by colony PCR and phenotypic checks, and integrating the next lox site flanked marker into a new locus. For example, a strain can harbor knockouts in modifications that allow production from glucose (GAL80 knockout in combination with either MIG1 or GAL1 knockouts), knockouts in genes involved in hexanoic acid or hexanoyl-CoA degradation (e.g. FAA2 and ANT1 knockouts), and/or knockouts in one or multiple proteases involved in degradation of expressed heterologous pathway proteins (e.g. PRB1 and PEP4 knockouts).

### Example 1G: Small-Scale Strain Screening Examples

Strains were tested either in shake flasks or an adapted protocol scaling down to 96 well plates. For shake flask testing, precultures were grown overnight (approximately 16-24 hours) in 15 or 30 mL of YP+2% (w/v) glucose in 250 mL baffled shake flasks at 30° C. with 200 rpm shaking and 80% humidity. Main cultures were inoculated using between 1 to 3 mL of preculture in 250 ml baffled shake flasks containing 30 ml of YP+0.02-0.04% (w/v) hexanoic acid+2% (w/v) galactose or 2% (w/v) glucose+5 ml of isopropyl myristate (IPM).

In some experiments, the percentage of galactose or glucose was altered, the percent hexanoic acid added was modified, or the overlay was intentionally not added or was replaced with alternative overlay candidates, such as diethyl sebacate, di-tert-butyl malonate, or methyl soyate. Sampling time was between 24 to 50 hours as indicated.

The shake flask experiments were scaled down to 96 well deepwell plate format. Precultures from colonies of each strain were grown in 300 μL YP+2% (w/v) glucose. Main cultures containing 300 μL YP+2% (w/v) galactose (or glucose or combinations of galactose and glucose)+0.04% (w/v) hexanoic acid+20% (v/v) IPM (60 μL) or alternative overlay candidates, were grown at 30° C. with 950 rpm shaking and 80% humidity in an Infors Multitron plate shaker, and the IPM or diethyl sebacate overlay was sampled at different elapsed times between 18 and 48 hours post-inoculation, following acidification of the media with 10 μL of 5 M phosphoric acid. Overlay from the cultures was diluted 2:1 with methanol prior to HPLC analysis.

Additional defined media optimization and production experiments were conducted with YNB or Delft medium base. YNB medium was initially optimized and consisted of 100 mL/L of a 10×YNB stock solution (containing 68 g/L yeast nitrogen base without amino acids from Sigma-Aldrich, product number Y0626), optionally 1 mL/L of 10% Bacto™ casamino acids (BD Biosciences), 300 mL/L of 1 M MES buffer (pH 6.5), optionally 3.6 mL/L of a trace element solution (containing 130 g/L citric acid monohydrate, 0.574 g/L copper (II) sulfate pentahydrate, 8.07 g/L iron (III) chloride hexahydrate, 0.5 g/L boric acid, 0.333 g/L manganese (II) chloride, 0.2 g/L sodium molybdate, and 4.67 g/L zinc sulfate heptahydrate), and optionally 1 mL/L of a vitamin solution (containing 0.008 g/L biotin, 1.6 g/L calcium pantothenate, 0.008 g/L folic acid, 8 g/L myo-inositol, 1.6 g/L nicotinic acid, 0.8 g/L p-aminobenzoic acid, 1.6 g/L pyridoxal hydrochloride, 0.8 g/L riboflavin, 1.6 g/L thiamine hydrochloride, adjusted to pH 10.5 with sodium hydroxide.

Delft CSM medium, consisted of (per liter solution) 7.5 g ammonium sulfate, 14.4 g potassium phosphate monobasic, 0.5 g magnesium sulfate heptahydrate (with these first three components prepared as an 0.9× solution and adjusted to pH 6.5 with sodium hydroxide prior to autoclaving), 3.6 mL of a trace metal solution (consisting of 130 g/L citric acid monohydrate, 0.574 g/L copper (II) sulfate pentahydrate, 8.07 g/L iron (III) chloride hexahydrate, 0.5 g/L boric acid, 0.333 g/L manganese (II) chloride, 0.2 g/L sodium molybdate, and 4.67 g/L zinc sulfate heptahydrate), 1.0 ml of a vitamin solution (0.008 g/L biotin, 1.6 g/L calcium pantothenate, 0.008 g/L folic acid, 8 g/L myo-inositol, 1.6 g/L nicotinic acid, 0.8 g/L p-aminobenzoic acid, 1.6 g/L pyridoxal hydrochloride, 0.8 g/L riboflavin, 1.6 g/L thiamine hydrochloride, adjusted to pH 10.5 with sodium hydroxide), 0.79 g of Complete Supplement Mixture (Formedium, Norfolk, UK), and 2% (w/v) of either galactose or glucose where specified. The final media was filter-sterilized, and hexanoic acid was added to 0.04% (w/v) for production.

Titers for both screening methods are presented as mg/L values on the basis of the entire volume of broth and overlay (total volume of 35 mL for shake flasks and 0.36 mL for 96 well deep well plates). In some plots, “olivetol equivalents” are depicted, which is the titer of olivetol, plus the titer of olivetolic acid multiplied by the molecular weight of olivetol divided by the molecular weight of olivetolic acid. In some plots, “olivetolic acid equivalents” are depicted, which is the titer of olivetolic acid, plus the titer of olivetol multiplied by molecular weight of olivetolic acid divided by the molecular weight of olivetol.

50 hour shake flask production of olivetolic acid and olivetol from LSC3-2 (“3×”), LSC3-4 (“3× gal80\hat{ }:: HIS4”), and LSC3-13 (“3× gal80\hat{ }:: HIS4 mig1\hat{ }”) from YP+2% (w/v) galactose+0.02% (w/v) hexanoic acid and YP+2% (w/v) glucose+0.02% (w/v) hexanoic acid for LSC3-2 were determined.

### Example 1H

50 hour 96 well deepwell plate production of olivetolic acid equivalents (total olivetolates) and corresponding measured optical density (600 nm) values from the aqueous phase of the culture for LSC3-2 (pink) and LSC3-4 (blue) cultivated in YP+2% or 4% (w/v) galactose+varying concentrations of hexanoic acid were determined. A tradeoff between hexanoic acid toxicity and conversion efficiency of hexanoic acid to end product could be observed, with an optimum between 0.08 to 0.1% (w/v) for 50 hour sampling. Lower concentrations of hexanoic acid can be employed to minimize cellular toxicity with earlier sampling points.

### Example 11

50 hour 96 well deepwell plate production of olivetolic acid equivalents (total olivetolates) and corresponding measured optical density (600 nm) values from the aqueous phase of the culture for, from left to right in each column, LSC3-13 (pink), LSC3-18 (green), LSC3-2 (blue), and LSC3-4 (purple) cultivated in YNB base medium plus 2% (w/v) galactose (“gal”) or 2% (w/v) glucose (“glu”), with or without casamino acids (“a.a”) or casamino acids plus trace element and vitamin solutions (“a.a_v.t”)+0.04% (w/v) hexanoic acid. Optimal production levels were observed in YNB medium supplemented with casamino acids and vitamin plus trace element solutions.

### Example 1J

Further defined media optimization with alternative defined amino acid compositions and vitamin/trace solutions for LSC3-4 and LSC3-18, with total olivetolate equivalents after 24 hours and optical density (600 nm), were determined. Glucose was added to 2% (w/v), galactose to 0.05% (w/v), and hexanoic acid to 0.04% (w/v). For these fully amino acid prototrophic strains, optimal growth and production were observed in Delft medium base containing CSM supplement and the trace and vitamin solution (T05 and V01) for which the composition is described above.

### Example 1K

18 hour and 48 hour 96 well deepwell plate titers of olivetolic acid and olivetol (and byproducts PDAL and HTAL) for strains LSC3-2, LSC3-48 and LSC3-49 (FAA2 knockouts in LSC3-2), LSC3-63 (PXA1 knockout in LSC3-2), LSC3-74 and LSC3-75 (PEX11 knockouts in LSC3-2), LSC3-76 and LSC3-77 (ANT1 knockouts in LSC3-2), and LSC3-47 (PRB1 knockout in LSC3-2) in YP medium+2% (w/v) galactose+0.04% (w/v) hexanoic acid+20% (v/v) IPM. The sampling time at 18 hours is indicative of productivity/rate of product formation due to hexanoic acid not yet being depleted. The sampling time at 48 hours represents a total conversion of hexanoic acid after hexanoic acid is fully utilized. For LSC3-48, LSC3-74, LSC3-76, and LSC3-77 in particular, both improved 18 and 48 hour titers were observed, indicating more efficient incorporation of hexanoic acid into olivetolic acid and olivetol. LSC3-48 and LSC3-76/77 had the highest overall conversions of hexanoic acid to olivetolic acid and olivetol, therefore these FAA2 and ANT1 were selected for further combinatorial knockouts and introduction into galactose independent strains. LSC3-47 had a higher 48 hour titer of olivetolic acid, indicating a potential role in proteolysis of CsOAC.

### Example 1L

24 hour and 48 hour 96 well deepwell plate titers of olivetolic acid and olivetol (and byproducts PDAL and HTAL) for strains LSC3-2, LSC3-50 and LSC3-51 (LSC3-2 his4::loxHIS4 as HIS4 prototrophic controls), LSC3-48 and LSC3-49 (FAA2 knockouts in LSC3-2), LSC3-77 (ANT1 knockout in LSC3-2), LSC3-47 (PRB1 knockout in LSC3-2), and LSC3-87 and LSC3-88 (PEP4 knockouts in LSC3-2) in YP medium+2% (w/v) galactose+0.04% (w/v) hexanoic acid +20% (v/v) IPM. The sampling time at 24 hours is indicative of productivity/rate of product formation due to hexanoic acid not yet being fully depleted. Higher 24 hour productivities were again observed for LSC3-48 and LSC3-77, indicating more efficient incorporation of hexanoic acid into olivetolic acid and olivetol. LSC3-87 and LSC3-88 also had higher 24 hour titers, than LSC3-2 or LSC3-50/51, indicating a higher pathway flux to olivetolic acid and olivetol from the PEP4 knockout. PEP4 and PRB1 are additionally selected for further combinatorial knockouts and introduction into galactose-independent strain.

### Example 1M

24 and 48 hour total olivetolate titers for strains LSC3-2, LSC3-4, LSC3-46, LSC3-18, and LSC3-64 and LSC3-65 (GAL80, MIG1, GAL1 triple knockout strains) tested in YP medium +2% (w/v) glucose+different indicated galactose concentrations (0, 0.05, 0.25, or 1.0% (w/v)). LSC3-64 and LSC3-65 combine the features of LSC3-13 and LSC3-18, with greatly enhanced productivities up to at least 24 hours compared to LSC3-13 in YP+2% glucose that are more similar to productivities from LSC3-18, as well as reducing the galactose-dependent inhibition of production of LSC3-13 after 48 hours.

### Example 1P

(Left) 24 and 48 hour total olivetolate titers for strains LSC3-13, LSC3-18, LSC3-89 and LSC3-90 (GPD1 knockouts in LSC3-13), and LSC3-91 and LSC3-92 (GPD1 knockouts in LSC3-18) in YP+2% (w/v) glucose+0.04% (w/v) hexanoic acid+20% (v/v) IPM (left side), or the same but with an additional 0.05% (w/v) galactose (right side). In the presence of 0.05% galactose, LSC3-89 and LSC3-90 have slightly increased final titers compared to LSC3-13. (Right) glycerol titers after 48 hours indicate greatly reduced glycerol formation in all GPD1 knockout strains.

### Example 1N

48 hour 96 well deepwell plate production of LSC3-2 in YP+2% (w/v) galactose+0.04% (w/v) hexanoic acid+20% (v/v) of different overlays (IPM, di-tert-butyl malonate, diethyl sebacate, and methyl soyate). Enhanced production was observed with the diethyl sebacate overlay compared to IPM.

### Example 2A: Generation of a Two Plasmid System for Screening Acyl Activating Enzyme (AAE) Activity

In order to perform in vivo screening in Saccharomyces cerevisiae for acyl activating enzyme (AAE) variants with enhanced activity and/or expression, a two plasmid screening system was employed to generate an artificial bottleneck in AAE activity. This bottleneck was generated by natural copy number variation of the two plasmids, with a multi-copy 2 micron episomal plasmid, pLOA-049, harboring an olivetol synthase homologue from C. sativa (CsTKS) fused with a T2A self-cleaving peptide (Kearsey et al., FEBS J. 287:1511-1524, 2020) to a gene encoding olivetol acid cyclase from C. sativa (CsOAC), and a lower copy centromeric plasmid with an autonomous replicating sequence (CEN/ARS) harboring different acyl activating enzymes/acyl-CoA synthetases from different organisms. Markers employed on the 2 micron plasmid were orotidine-5′-phosphate decarboxylase from S. cerevisiae (URA3) and on the CEN/ARS plasmid, the multifunctional histidine biosynthesis gene HIS4 from S. cerevisiae. The cassette architecture in the plasmids was based on integrated cassettes in production strain LSC3-2, with a pGAL1-10 bidirectional promoter driving expression of CsTKS and CsOAC from pGAL1 with a S. cerevisiae CYC1 terminator, and insulated downstream of pGAL10 by a S. cerevisiae GRE3 terminator. Likewise on the CEN/ARS plasmid, a bidirectional pGAL1-10 promoter drives expression of CsAAE1 from pGAL10 with a S. cerevisiae GRE3 terminator, and is insulated downstream of pGAL1 by a S. cerevisiae CYC1 terminator.

Plasmid pLOA-049 was constructed from four PCR fragments amplified using Q5 DNA polymerase (New England Biolabs) and a touchdown annealing thermal cycler protocol. One 184 base pair fragment containing the S. cerevisiae GRE3 terminator sequence was amplified from internal plasmid s991 using primers YO11578 and YO6760. A 3685 base pair and 2259 base pair fragment containing the 2 micron replication origin and URA3 marker were amplified from internal plasmid pLOA-020 using primers YO11497 and YO11575, and YO11580 and YO11393, respectively. The remainder of the pathway cassette minus CsAAE1 was amplified as a 2591 base pair fragment from pLOA-006 using primers YO11576 and YO11581. All of these fragments were assembled into the final plasmids by sequence- and ligation-independent cloning (SLIC) by mixing normalized quantities of fragments in a 10 μL reaction with 0.25 μL T4 DNA polymerase (New England Biolabs) and 1 μL Buffer 2.1 (New England Biolabs) on ice, incubating at room temperature for 5 minutes, returning to ice, and transforming the entire mixture into E. coli DH10B chemically competent cells. The pathway cassette in pLOA-049 was fully sequence validated by Sanger sequencing.

Plasmid pLOA-059, containing AAE1 from C. sativa codon-optimized for S. cerevisiae (CsAAE1), CEN/ARS, and a HIS4 marker, was used as a control in activity screens for other homologues. It was constructed from two PCR fragments amplified as described above, assembling a 6851 base pair fragment from pLOA-054 containing the HIS4 marker and CEN/ARS using primers YO11669 and YO12052, and a 3082 base pair fragment from pLOA-046 containing the pathway cassette minus CsTKS and CsOAC using primers YO12051 and YO12050. Plasmid pLOA-054 (empty vector with CEN/ARS and HIS4 marker) was constructed by assembling two PCR fragments: one 3020 base pair fragment containing the HIS4 marker amplified from S. cerevisiae CEN.PK2-1C MATa genomic DNA with primers YO11657 and YO11659, and one 4561 base pair fragment containing CEN/ARS and an empty expression cassette with an S. cerevisiae ADH2 promoter and S. cerevisiae CYC1 terminator, from internal plasmid pLOA-016 using primers YO11660 and YO11658. Plasmid pLOA-046 (yeast integrating plasmid where a full pathway cassette with the S. cerevisiae GRE3 terminator was first cloned) was constructed from three PCR fragments as described above, with a 219 base pair fragment containing the S. cerevisiae GRE3 terminator sequence amplified from internal plasmid s991 using primers YO11655 and YO11578, a 4083 base pair fragment containing part of the pathway cassette between the T2A sequence following CsTKS and the end of CsAAE1 amplified from pLOA-006 using primers YO11654 and YO9947, and a 4939 base pair fragment containing CsOAC, the CYC1 terminator, E. coli origin and ampicillin resistance marker, and a URA3 gene cassette from S. cerevisiae from plasmid pLOA-007 using primers YO9942 and YO11653. The pathway cassette in pLOA-059 was fully sequence validated by Sanger sequencing.

Plasmid pLOA-327 contains acyl activating enzyme 7 (AAE7) from Arabidopsis thaliana (Uniprot entry Q8VZF1) codon-optimized for S. cerevisiae and with the C-terminal peroxisomal targeting sequence (PTS1) removed to enable cytosolic expression (AtAAE7). The gene was originally synthesized and cloned into an insulated low copy (p15A origin) E. coli vector by Twist Bioscience, tw949. Plasmid pLOA-327 was constructed from two PCR fragments amplified as described previously, assembling a 1778 base pair fragment containing the AtAAE7 gene from tw949 using primers YO12300 and YO12280, and a 7741 base pair fragment containing the remaining CEN/ARS HIS4 expression plasmid backbone, including the pGAL1-10 promoter, CYC1 terminator, and GRE3 terminator from pLOA-059 using primers YO12279 and YO12297. The pathway cassette was fully validated by Sanger sequencing.

Plasmid pLOA-333 contains 4-coumarate--CoA ligase-like 6 (4CLL6) from Arabidopsis thaliana (Uniprot entry Q84P24) codon-optimized for S. cerevisiae (At4CLL6). The gene was originally synthesized and cloned as described for AAE7 into a vector, tw956. Plasmid pLOA-333 was constructed from two PCR fragments amplified as described previously, assembling a 1762 base pair fragment containing At4CLL6 using primers YO12311 and YO12312, and the same 7741 base pair backbone fragment described for pLOA-327. The pathway cassette was fully validated by Sanger sequencing.

A background strain to accommodate transformation of the two plasmid system (with HIS4 and URA3 markers), and enabling galactose-independent induction of the pGAL1-10 promoter via knockouts in the GAL80 and MIG1 loci, LSC3-297, was generated. This strain possesses only a histidine auxotrophy via a defective HIS4 and uracil auxotrophy via a defective URA3.

### Example 2B: Use of the Two Plasmid Screening System to Assess Relative AAE Activity

To screen activity of AAE variants, first a CEN/ARS containing plasmid expressing AAEs co-transformed with the 2 micron plasmid expressing CsTKS translationally fused to CsOAC via the T2A self-cleaving peptide into strain LSC3-297 using the transformation method described by Gietz and Schiestl (Nat. Protocols 2:31-34, 2007). Double plasmid transformants were selected on YNB agar with 2% (w/v) glucose and CSM minus histidine and minus uracil (CSM-His-Ura, Sunrise Science Products) and grown in a 30° C. incubator for approximately 48 hours or at room temperature for approximately 72 hours. Individual colonies were picked into wells of 96 deepwell plates containing 0.3 mL per well of Delft medium with 2% (w/v) glucose and 0.75 g/L CSM-His-Ura. Delft medium base contains (per liter solution) 7.5 g ammonium sulfate, 14.4 g potassium phosphate monobasic (added from a 1 M stock solution adjusted to pH 6.5 with sodium hydroxide), 0.5 g magnesium sulfate heptahydrate, 3.6 mL of a trace metal solution (consisting of 130 g/L citric acid monohydrate, 0.574 g/L copper (II) sulfate pentahydrate, 8.07 g/L iron (III) chloride hexahydrate, 0.5 g/L boric acid, 0.333 g/L manganese (II) chloride, 0.2 g/L sodium molybdate, and 4.67 g/L zinc sulfate heptahydrate), and 1.0 ml of a vitamin solution (0.008 g/L biotin, 1.6 g/L calcium pantothenate, 0.008 g/L folic acid, 8 g/L myo-inositol, 1.6 g/L nicotinic acid, 0.8 g/L p-aminobenzoic acid, 1.6 g/L pyridoxal hydrochloride, 0.8 g/L riboflavin, 1.6 g/L thiamine hydrochloride, adjusted to pH 10.5 with sodium hydroxide). Plates were grown for 16-24 hours at 30° C. in a plate shaker incubator maintained at 80% relative humidity and 900 rpm shaking (3 mm diameter throw). Ten microliters of preculture was then used to inoculate wells of 96 deepwell plates containing 0.3 mL per well of Delft medium with 2% (w/v) glucose, 0.75 g/L CSM-His-Ura, either 9.08 mM butyric acid or 3.44 mM hexanoic acid, and for divarin/divarinic acid, additionally 150 mM 2-(N-morpholino) ethanesulfonic acid (MES) at pH 5.5 and with the additional modification of the 1 M potassium phosphate monobasic stock in the Delft medium base also adjusted to pH 5.5 (for olivetol/olivetolic acid production from hexanoic acid, no MES was added and potassium phosphate monobasic solution used in the Delft medium base was at pH 6.5). Plates were grown for 48 hours at 30° C. in a plate shaker incubator maintained at 80% relative humidity and 900 rpm shaking (3 mm diameter throw). To quantify the total amount of products (divarinic acid and divarin, or olivetolic acid and olivetol) formed in each culture, cells were spun down via centrifugation and aqueous supernatants were passed through an 0.22 micron filter plate into an HPLC collection plate (Waters). Plates were sealed with aluminum foil on a PlateLoc plate sealer (Agilent Technologies) and analyzed by a reverse-phase HPLC method with UV detection at 235 nm absorption for divarin, divarinic acid, olivetol, and olivetolic acid. Concentrations were calculated using a standard curve generated from purchased divarin, divarinic acid, olivetol, and olivetolic acid standards.

### Example 2C: Comparison of Activity of AAE Homologues

LSC3-297/pLOA-049/pLOA-059, LSC3-297/pLOA-049/pLOA-327, and LSC3-297/pLOA-049/pLOA-333, expressing CsAAE1, AtAAE7, and At4CLL6, respectively, were tested for production of divarin and divarinic acid, and olivetol and olivetolic acid by the method described in Example 2B. The results are shown in Table 2 for production of both olivetol and olivetolic acid with hexanoic acid feeding, and divarin and divarinic acid from butyric acid feeding.

Table 2: Average olivetol and olivetolic acid production, and divarin and divarinic acid production from two plasmid screening system strains expressing different AAEs in Delft medium (pH 6.5 for olivetol/olivetolic acid, pH 5.5 with 150 mM MES for divarin/divarinic acid)+2% (w/v) glucose+CSM-His-Ura+3.44 mM hexanoic acid (for olivetol/olivetolic acid) or 9.08 mM butyric acid (for divarin/divarinic acid).

| | | | Olivetolic | | Divarinic |
|---|---|---|---|---|---|
| | | Olivetol | acid | Divarin | acid |
| Strain | AAE | (mg/L) | (mg/L) | (mg/L) | (mg/L) |
| LSC3-297/pLOA- | CsAAE1 | 2.61 | 2.99 | 1.58 | 1.51 |
| 049/pLOA-059 | | | | | |
| LSC3-297/pLOA- | AtAAE7 | 0.74 | 1.60 | 7.18 | 5.63 |
| 049/pLOA-327 | | | | | |
| LSC3-297/pLOA- | At4CLL6 | 0.86 | 0.91 | 5.96 | 5.24 |
| 049/pLOA-333 | | | | | |

Out of the tested AAE sequences, CsAAE1 has the highest activity as a hexanoyl-CoA synthetase for olivetol and olivetolic acid production, while AtAAE7 and At4CLL6 exhibit several-fold activity improvements as butyryl-CoA synthetases for divarin and divarinic acid production over CsAAE1.

### Fermentation Examples

### Example 3: Divarinic Acid/Divarin Production in LSC3-2 and Derived Strains in Defined Media with Varying pH

A.
Strain: LSC3-134
Genotype: JK9-3d MATa gal80Δ::lox72 mig1Δ::lox66-PkHIS4-lox71 TRP1::pLOA-006 URA3::pLOA-007 LEU2::pLOA-019

### Fermentation Process Summary:

### Seed Train:

A shake flask containing 50 mL YPD with 20 g/L glucose was inoculated with a seed vial containing LSC3-134. Strain grew at 30 C for 24 hours to an OD600 of 3-4. 15 ml of this culture was used to inoculate the fermentation tank.

### Media:

Seed Media: YP with 20 g/L glucose
Batch media:
10 g/L yeast extract+20 g/L peptones+20 g/L glucose+500 mg/L histidine+12 mg/L myo-inositol+12 mg/L thiamin hydrochloride+12 mg/L pyridoxal hydrochloride+12 mg/L nicotinic acid+12 mg/L calcium pantothenate+0.6 mg/L biotin+12 mg/L p-aminobenzoic acid+0.15 g/L EDTA+7.8 mg/L CuSO4-5H2O+0.0512 g/L FeSO4-7H2O+0.0032 g/L MnCl2+4.77 mg/L Na2MoO4+0.102 g/L ZnSO4-7H2O+0.0086 g/L CoCl2-6H2O+0.0384 g/L CaCl₂)-2H2O+5.5 g/L KH2PO4+2.9 g/L MgSO4-7H2O+45.1 g/L (NH4)2SO₄

### Production Media:

650 g/L glucose+438 mg/L citric acid monohydrate+2 mg/L H3BO3+1.3 mg/L CuSO4-5H2O+22.4 mg/L FeCl3-6H2O+1.33 mg/L MnCl2+0.8 mg/L Na2MoO4+10.8 mg/L ZnSO4-7H2O+12 mg/L myo-inositol+12 mg/L thiamin hydrochloride+12 mg/L pyridoxal hydrochloride+12 mg/L nicotinic acid+12 mg/L calcium pantothenate+12 mg/L biotin+12 mg/L p-aminobenzoic acid+12 mg/L folic acid+12 mg/L riboflavin+2.5 g/L KH2PO4+1 g/L MgSO4-7H2O+20 g/L (NH4)2SO4+20 g/L sodium butyrate

### Base (for pH Control)

5M NH₄OH

### Overlay

170 mL isopropyl myristate (40% V/VO) was added to tank at 24 hours.
Antifoam Struktol SB2121 (0.1 mL/L) at the beginning of the run

### Fermentation Run Condition:

We used pulse feeding during the run. Fermentation batch was inoculated with 15 ml of inoculum. Feeding was triggered at the end of batch phase when batch glucose was completely exhausted and pO2 was increased by 10% (or more). Feed media (growth or production media) was delivered in pulses. Each pulse delivered 1.7 g glucose/starting batch volume with maximum feed rate not exceeding 10 g glucose/L/hr. pH was maintained at 5.5 throughout the run. Temperature of fermenter was maintained at 30C. Air flow rate was maintained at 1.25 L/min. Agitation was 800 rpm.

Summary of metrics:
Maximum Divarinic acid titer: 2.5 g/L
Maximum Divarin titer: 2.5 g/L
Total product titer: 5 g/L (D+DA)
Titer/time: 0.74 g Divarin equivalent/L/day

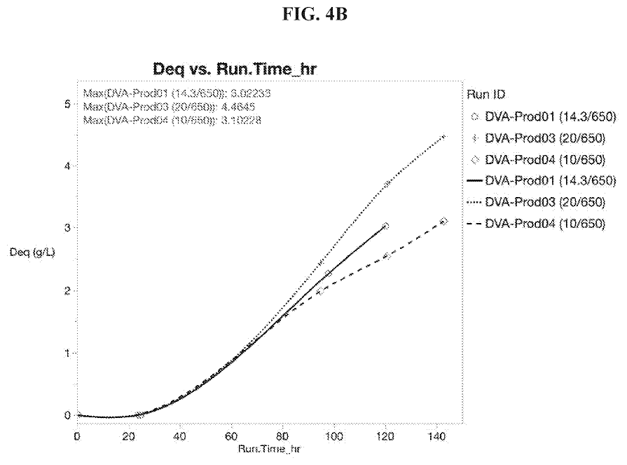

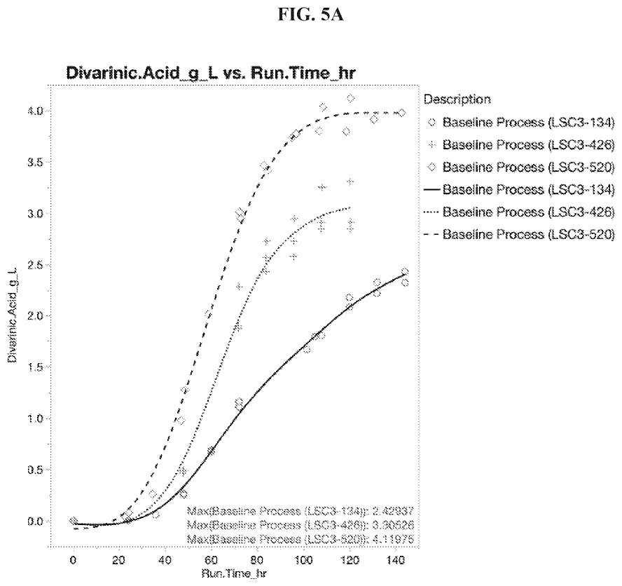

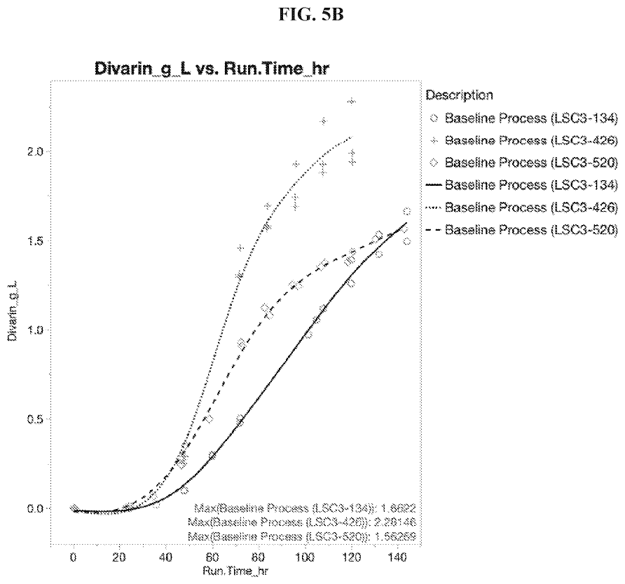

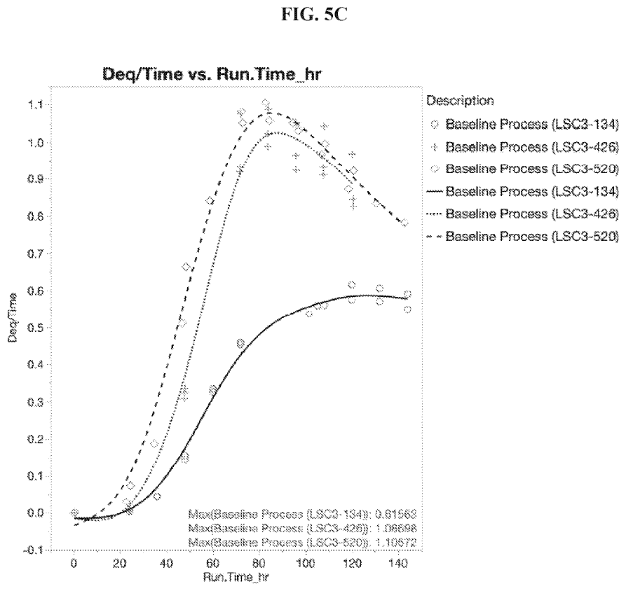

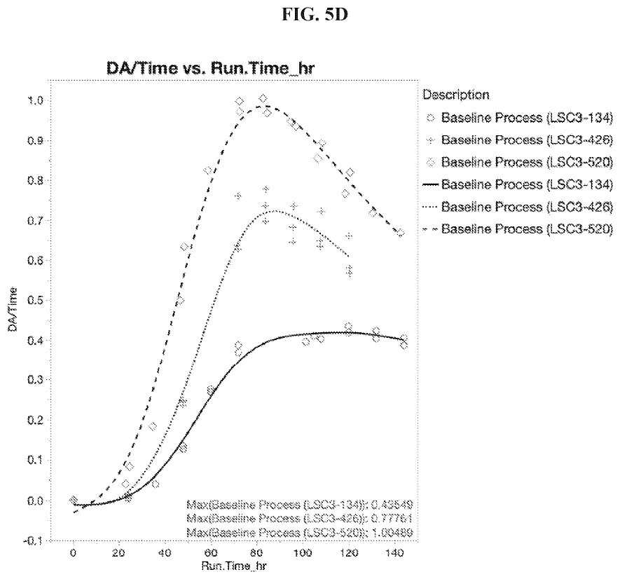

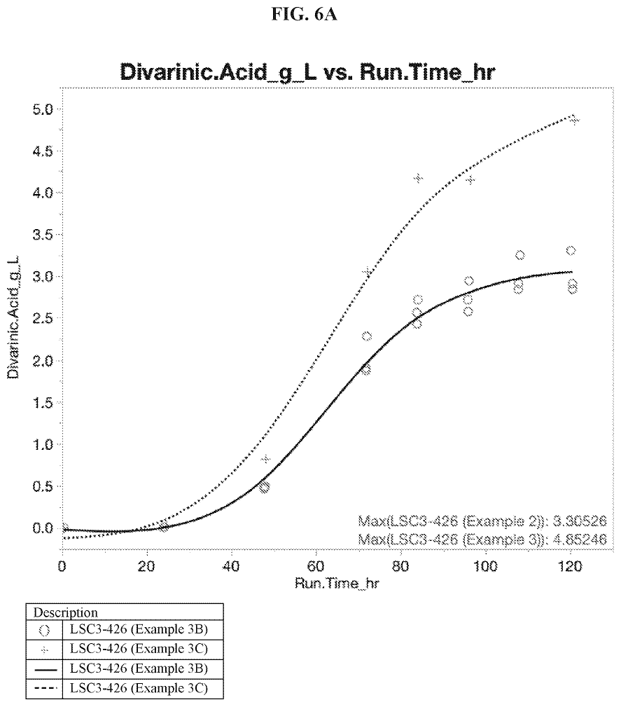

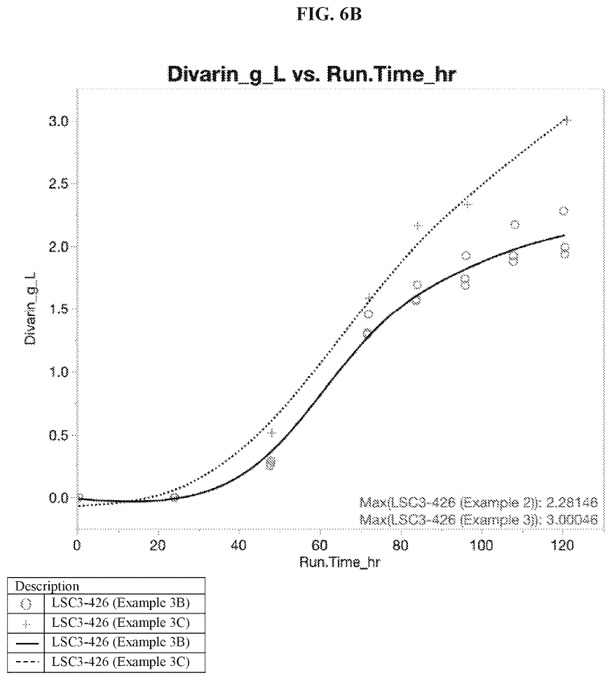

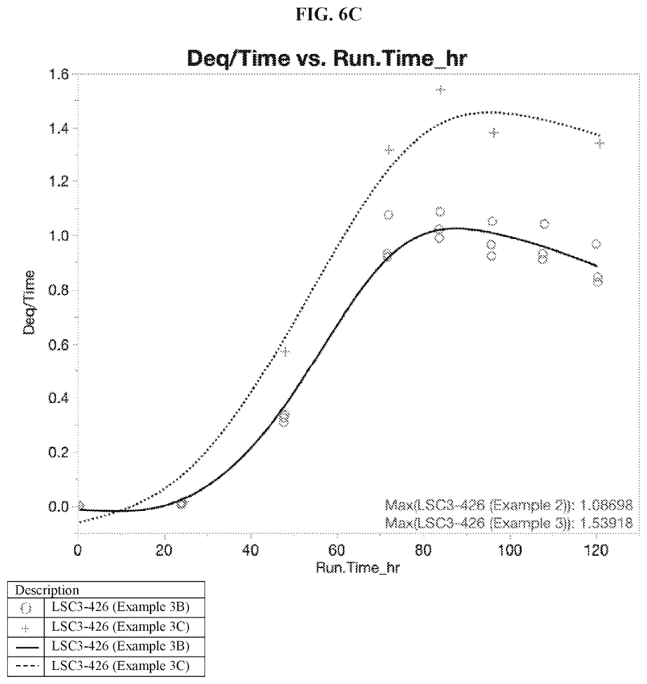

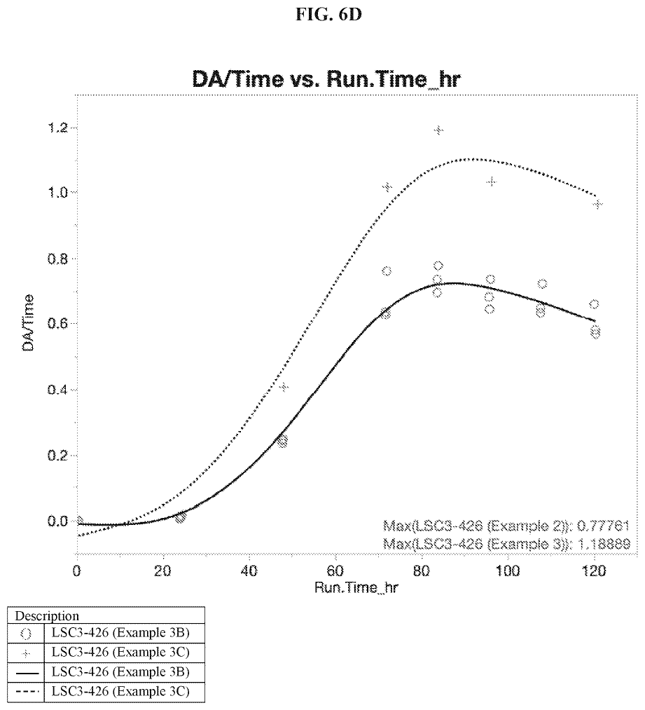

We also evaluated the effect of sodium butyrate concentration in feed in a set of three runs (10, 14.3 and 20 g/L sodium butyrate in feed, all other nutrients remained the same as stated above) and we observed that titer and productivity increased as we increased sodium butyrate concentration in feed. The highest titer (and productivity) was observed in the run with 20 g/L sodium butyrate in feed. The results are graphically illustrated in FIGS. 4A-4B.

B.
Strain: LSC3-134, LSC3-426, LCS3-520
Genotype (LSC3-134): JK9-3d MATa gal80Δ::lox72 mig1Δ::lox66-PkHIS4-lox71 TRP1::pLOA-006 URA3::pLOA-007 LEU2::pLOA-019
Genotype (LSC3-426): JK9-3d MATa gal80Δ::lox72 mig1Δ::lox72 TRP1::pLOA-006 URA3::pLOA-007 LEU2::pLOA-019 HIS4::pLOA-419
Genotype (LCS3-520): JK9-3d MATa gal80Δ::lox72 mig1Δ::lox72 trp1Δ:: TRP1 (S288c) leu2Δ::LEU2 (S288c) his4Δ:: HIS4 (CEN.PK2-1Ca) ura3Δ::lox66-HygR-lox71-(KIURA3-K28*-deg) KIURA3-K28*-deg::pLOA-516

### Fermentation Process Summary:

### Seed Train:

A shake flask containing 50 mL YPD with 20 g/L glucose was inoculated with a seed vial containing LSC3-134, LSC3-426 or LSC3-520. Strains grew at 30 C for 24 hours to an OD600 of 10-12. 3.5 mL of this culture was used to inoculate the fermentation tank.

### Media:

Seed Media: YP with 20 g/L Glucose
Batch media:
10 g/L yeast extract+20 g/L peptones+20 g/L glucose+500 mg/L histidine+12 mg/L myo-inositol+12 mg/L thiamin hydrochloride+12 mg/L pyridoxal hydrochloride+12 mg/L nicotinic acid+12 mg/L calcium pantothenate+0.6 mg/L biotin+12 mg/L p-aminobenzoic acid+0.15 g/L EDTA+7.8 mg/L CuSO4-5H2O+0.0512 g/L FeSO4-7H2O+0.0032 g/L MnCl2+4.77 mg/L Na2MoO4+0.102 g/L ZnSO4-7H2O+0.0086 g/L CoCl2-6H2O+0.0384 g/L CaCl2-2H2O+5.5 g/L KH2PO4+2.9 g/L MgSO4-7H2O+45.1 g/L (NH4)2SO4

### Production Media:

500.5 g/L glucose+337.26 mg/L citric acid monohydrate+1.54 mg/L H3BO3+1 mg/L CuSO4-5H2O+17.25 mg/L FeCl3-6H2O+1 mg/L MnCl2+0.62 mg/L Na2MoO4+8.32 mg/L ZnSO4-7H2O+9.24 mg/L myo-inositol+9.24 mg/L thiamin hydrochloride+9.24 mg/L pyridoxal hydrochloride+9.24 mg/L nicotinic acid+9.24 mg/L calcium pantothenate+9.24 mg/L biotin+9.24 mg/L p-aminobenzoic acid+9.24 mg/L folic acid+9.24 mg/L riboflavin+1.93 g/L KH2PO4+0.77 g/L MgSO4-7H2O+15.4 g/L (NH4)2SO4+11.03 g/L sodium butyrate

### Base (for pH Control)

5M NH₄OH

### Overlay

38 mL isopropyl myristate (38% V/VO) was added to tank at 24 hours.
Antifoam Struktol SB2121 (0.1 mL/L) at the beginning of the run

### Fermentation Run Condition:

We used pulse feeding during the run. Fermentation batch was inoculated with 3.5 ml of inoculum (3.5% V/VO). Feeding was triggered at the end of batch phase when batch glucose was completely exhausted and pO2 was increased by 10% (or more). Feed media (production media) was delivered in pulses. Each pulse delivered 1.7 g glucose/starting batch volume with maximum feed rate not exceeding 10 g glucose/L/hr. pH was maintained at 5.5 throughout the run. Temperature of fermenter was maintained at 30C. Air flow rate was maintained at 100 mL/min. Agitation was 2000 rpm.

### Summary of Metrics:

LSC3-134:
   Maximum Divarinic acid: 2.4 g/L
   Maximum Divarin: 1.7 g/L
   Titer/time: 0.7 g Divarin equivalent/L/day
   (Divarinic acid) Titer/time: 0.47 g/L/day
LSC3-426:
   Maximum Divarinic acid: 3.3 g/L
   Maximum Divarin: 2.3 g/L
   Titer/time: 1.09 g Divarin equivalent/L/day
   (Divarinic acid) Titer/time: 0.78 g/L/day
LSC3-520:
   Maximum Divarinic acid: 4.1 g/L
   Maximum Divarin: 1.6 g/L
   Titer/time: 1.1 g Divarin equivalent/L/day
   (Divarinic acid) Titer/time: 1 g/L/day

The results are graphically illustrated in FIGS. 5A-5D.

C.
Strain: LSC3-426
Genotype (LSC3-426): JK9-3d MATa gal80Δ::lox72 mig1Δ::lox72 TRP1::pLOA-006 URA3::pLOA-007 LEU2::pLOA-019 HIS4::pLOA-419

### Fermentation Process Summary:

### Seed Train:

A shake flask containing 50 mL YPD with 20 g/L glucose was inoculated with a seed vial containing LSC3-426. Strains grew at 30 C for 24 hours to an OD600 of 10-12. 3.5 ml of this culture was used to inoculate the fermentation tank.

### Media:

Seed Media: YP with 20 g/L glucose
Batch media:
10 g/L yeast extract+20 g/L peptones+20 g/L glucose+500 mg/L histidine+12 mg/L myo-inositol+12 mg/L thiamin hydrochloride+12 mg/L pyridoxal hydrochloride+12 mg/L nicotinic acid+12 mg/L calcium pantothenate+0.6 mg/L biotin+12 mg/L p-aminobenzoic acid+0.15 g/L EDTA+7.8 mg/L CuSO4-5H2O+0.0512 g/L FeSO4-7H2O+0.0032 g/L MnCl2+4.77 mg/L Na2MoO4+0.102 g/L ZnSO4-7H2O+0.0086 g/L CoCl2-6H2O+0.0384 g/L CaCl2-2H2O+5.5 g/L KH2PO4+2.9 g/L MgSO4-7H2O+45.1 g/L (NH4)2SO4

### Production Media:

500.5 g/L glucose+337.26 mg/L citric acid monohydrate+1.54 mg/L H3BO3+1 mg/L CuSO4-5H2O+17.25 mg/L FeCl3-6H2O+1 mg/L MnCl2+0.62 mg/L Na2MoO4+8.32 mg/L ZnSO4-7H2O+9.24 mg/L myo-inositol+9.24 mg/L thiamin hydrochloride+9.24 mg/L pyridoxal hydrochloride+9.24 mg/L nicotinic acid+9.24 mg/L calcium pantothenate+9.24 mg/L biotin+9.24 mg/L p-aminobenzoic acid+9.24 mg/L folic acid+9.24 mg/L riboflavin+1.93 g/L KH2PO4+0.77 g/L MgSO4-7H2O+15.4 g/L (NH4)2SO4+17.33 g/L sodium butyrate

### Base (for pH Control)

5M NH₄OH

### Overlay

38 mL isopropyl myristate (38% V/VO) was added to tank at 24 hours.
Antifoam Struktol SB2121 (0.1 mL/L) at the beginning of the run

### Fermentation Run Condition:

We used pulse feeding during the run. Fermentation batch was inoculated with 3.5 ml of inoculum (3.5% V/VO). Feeding was triggered at the end of batch phase when batch glucose was completely exhausted and pO2 was increased by 10% (or more). Feed media (production media) was delivered in pulses. Each pulse delivered 1.07 g glucose/starting batch volume with maximum feed rate not exceeding 6.4 g glucose/L/hr. PH was maintained at 5.5 throughout the run. Temperature of fermenter was maintained at 30C. Air flow rate was maintained at 100 mL/min. Agitation was 2000 rpm.

### Summary of Metrics:

LSC3-426:
   Maximum Divarinic acid: 4.85 g/L
   Maximum Divarin: 3.0 g/L
   Titer/time: 1.54 g Divarin equivalent/L/day
   (Divarinic acid) Titer/time: 1.2 g/L/day

The results are graphically illustrated in FIGS. 6A-6D

### Example 4A: Olivetolic Acid and Olivetol Production with Organic Solvent Overlays

Precultures of LSC3-2 and LSC3-18 were inoculated from YPD streak plates and grown in 30 ml of YP+2% (w/v) glucose in baffled 250 ml shake flasks for approximately 18 hours overnight at 30° C. with 200 rpm shaking. Baffled 250 ml shake flasks containing 30 mL of YP+2% (w/v) galactose, 5 mL of IPM or diethyl sebacate, and 0.04% (w/v) hexanoic acid, were inoculated with 1 mL of preculture and grown for at 30° C. with 200 rpm shaking. After 24 and 48 hours, samples of cell culture broth plus overlay were sampled into microcentrifuge tubes and stored at −20° C. at least overnight. Sample tubes were thawed and aqueous sample and overlay sample were pipetted into plates for HPLC analysis. Overlay samples were diluted 1:1 v/v with methanol in the HPLC plate prior to analysis.

Total olivetol equivalents are defined as the concentration of olivetol in mg/L, plus the concentration of olivetolic acid in mg/L multiplied by the ratio of the molecular weight of olivetol to olivetolic acid. Lower total olivetol equivalents were observed with diethyl sebacate overlayer as compared to IPM. With IPM overlay, OA partitioned between the IPM and aqueous phases, with a substantial amount of OA remaining in the aqueous phase in these culturing conditions. By contrast, OA entirely partitioned into diethyl sebacate with none present in the aqueous phase. The reduction in total production levels with a diethyl sebacate overlay may occur due to a reduction in OD600 due to moderately growth inhibitory properties of diethyl sebacate.

In another experiment, LSC3-2 precultures were inoculated from YPD streak plates and grown in 300 μL YP+2% (w/v) glucose in round-bottom square well 96 well deepwell plates for approximately 18 hours overnight at 30° C. with 950 rpm shaking in an Infors plate shaker. 96 well deepwell plate wells containing 300 μL of YP+2% (w/v) galactose, 60 μL of IPM, diethyl sebacate, di-tert-butyl malonate, or methyl soyate, and 0.04% (w/v) hexanoic acid, were inoculated with 10 μL of preculture and grown for 30° C. with 950 rpm shaking. After 48 hours, cultures were acidified with 10 μL of 5 M phosphoric acid to enhance partitioning of olivetolic acid into the organic phase (as the free acid), and overlays were sampled on a Bravo automated liquid handling platform (Agilent) by first adding 120 μL of IPM, mixing on a shaking platform for several minutes, centrifuging the plate at 3000 rpm for 5 minutes to separate phases, and pipetting 100 μL of overlay from each well into an HPLC plate. Overlay samples were diluted 1:1 v/v with methanol in the HPLC plate, sealed and analyzed by HPLC.

Under these conditions, higher production levels were observed with a diethyl sebacate overlayer as compared with IPM. No product was observed in the overlayer (aqueous samples were not measured) with a di-tert-butyl malonate overlayer. Substantial production was observed in methyl soyate, however at slightly lower levels than with IPM.

In another experiment, several monoester overlay candidates and one diester candidate were compared to IPM. LSC3-13 precultures were inoculated from YPD streak plates and grown in 300 μL Delft medium+0.79 g/L complete supplement mixture (CSM) (ForMedium, Norfolk, UK)+2% (w/v) glucose in round-bottom square well 96 well deepwell plates for approximately 18 hours overnight at 30° C. with 950 rpm shaking in an Infors plate shaker. 96 well deepwell plate wells containing 300 μL of Delft medium+CSM+2% (w/v) glucose, 60 μL of IPM, diethyl sebacate, di-tert-butyl malonate, or methyl soyate, and 0.04% (w/v) hexanoic acid, were inoculated with 10 μL of preculture and grown for 30° C. with 950 rpm shaking. Delft medium contains (per liter solution) 7.5 g ammonium sulfate, 14.4 g potassium phosphate monobasic (added from a 1 M stock solution adjusted to pH 6.5 with sodium hydroxide), 0.5 g magnesium sulfate heptahydrate, 3.6 mL of a trace metal solution (consisting of 130 g/L citric acid monohydrate, 0.574 g/L copper (II) sulfate pentahydrate, 8.07 g/L iron (III) chloride hexahydrate, 0.5 g/L boric acid, 0.333 g/L manganese (II) chloride, 0.2 g/L sodium molybdate, and 4.67 g/L zinc sulfate heptahydrate), and 1.0 ml of a vitamin solution (0.008 g/L biotin, 1.6 g/L calcium pantothenate, 0.008 g/L folic acid, 8 g/L myo-inositol, 1.6 g/L nicotinic acid, 0.8 g/L p-aminobenzoic acid, 1.6 g/L pyridoxal hydrochloride, 0.8 g/L riboflavin, 1.6 g/L thiamine hydrochloride, adjusted to pH 10.5 with sodium hydroxide). After 48 hours, the aqueous layer and overlay were sampled on a Bravo automated liquid handling platform (Agilent) by first removing 200 μl of aqueous sample into a 96 well filter plate, adding 180 μL of IPM to each well, mixing on a shaking platform for several minutes, centrifuging the plate at 3000 rpm for 5 minutes to separate phases, and pipetting 100 μL of overlay from each well into an HPLC plate. Overlay samples were diluted 1:1 v/v with methanol in the HPLC plate, sealed and analyzed by HPLC. Aqueous samples were centrifuged in the 96 well filter plate at 3000 rpm for 5 minutes into an HPLC plate, sealed, and analyzed by HPLC.

Titers were calculated in each phase on the basis of the volume of the full broth plus overlay, thus concentrations reported correspond to actual concentrations in the full liquid volume of each production well. Ethyl myristate exhibited approximately equal aqueous phase concentrations of olivetolic acid and olivetol product as IPM, but with slightly higher overlay concentrations. Other monoesters also supported robust production slightly lower than that of IPM, including methyl decanoate and hexyl hexanoate. The results demonstrate that monoester overlays that are not inhibitory to growth support robust production of olivetolic acid and olivetol.

### Example 4B: Divarinic Acid and Divarin Production with Organic Solvent Overlays

Several monoester and one diester overlay candidate were compared to IPM for production of divarinic acid and divarin. LSC3-13 precultures were inoculated from YPD streak plates and grown in 300 μL Delft medium+0.79 g/L complete supplement mixture (CSM) (ForMedium, Norfolk, UK)+2% (w/v) glucose in round-bottom square well 96 well deepwell plates for approximately 18 hours overnight at 30° C. with 950 rpm shaking in an Infors plate shaker. 96 well deepwell plate wells containing 300 μL of Delft medium+CSM+2% (w/v) glucose, 60 μL of different overlay solvents, and 0.08% (w/v) butyric acid, were inoculated with 10 μL of preculture and grown for 30° C. with 950 rpm shaking. After 48 hours, the aqueous layer and overlay were sampled on a Bravo automated liquid handling platform (Agilent) and samples from the aqueous and organic overlay phases were subjected to HPLC analysis to measure divarinic acid and divarin production.

Multiple overlays support production of divarinic acid and divarin. Under the test conditions, divarinic acid and divarin partition less effectively into monoester overlays than olivetolic acid and olivetol. IPM allowed for higher production levels than other tested monesters without branched chain substituents.

### Example 5: Surprising Efficacy of Saccharomyces Cerevisiae JK9-3d for Cannabinoid Production

To generate Saccharomyces cerevisiae strains for production of the cannabinoid compounds olivetolic acid and divarinic acid, the strain JK9-3d was employed. In comparison to the S. cerevisiae strain CEN.PK2-1C, integrations into S. cerevisiae JK9-3d were surprisingly more efficient and resulted in higher titers of olivetolic acid and divarinic acid.

### Genetic Construct Design

Genetic constructs for olivetolic acid (OA) and divarinic acid (DA) production were built containing the genes Cannabis sativa acyl activating enzyme 1 (CsAAE1), Cannabis sativa tetraketide synthase (CsTKS), and Cannabis sativa olivetolic acid cyclase (CsOAC) for OA (pLOA-519) and Arabidopsis thaliana acyl activating enzyme 7 (AtAAE7), CsTKS, and CsOAC for DA (pLOA-520). Genes in pLOA-519 are expressed behind the pGAL1,10 bidirectional promoter with pGAL1 driving bicistronic expression of CsTKS and CsOAC fused with a 2A self cleaving peptide from thosea asigna virus (T2A) and pGAL10 driving expression of CsAAE1. Expression of CsOAC and CsAAE1 are insulated by the CYC1 and GRE terminators respectively. Genes in pLOA-520 are similarly expressed behind the pGAL1,10 bidirectional promoter with pGAL1 driving bicistronic expression of CsTKS and CsOAC fused with a 2A self cleaving peptide from thosea asigna virus (T2A) and pGAL10 driving expression of AtAAE7. Expression of CsOAC and AtAAE7 are insulated by the CYC1 and GRE terminators respectively.

pLOA-519 and pLOA-520 both contain the marker gene orotidine-5′-phosphate decarboxylase from Kluyveromyces lactis (KIURA3) fused to a degradation tag for selection on uracil dropout media. KIURA3 is expressed behind its native promoter pKIURA3 and insulated by its native terminator tKIURA3. All genes described above are flanked by retrotransposon TY4 homologous regions to facilitate multi-integrations into the S. cerevisiae genome. Plasmids pLOA-519 and pLOA-520 both contain a CEN/ARS origin and ScTRP1 marker for maintenance and selection in S. cerevisiae and a pUC origin and Ampicillin resistance marker for maintenance and selection in E. coli.

### Building Genetic Constructs

pLOA-519 was built using transformation associated recombination (TAR) cloning in S. cerevisiae CEN.PK-1c. Fragments for assembly were amplified from various sources using Q5 DNA polymerase. Fragments from plasmid sources were digested for 1 h with FastDigest DpnI (ThermoFisher Scientific) and column purified. Equimolar concentrations of each fragment were mixed, transformed into CEN.PK2-1C MATa, his3D1, leu2-3,112, ura3-52, trp1-289 using a lithium acetate transformation method (Nat. Protocols 2:31-34, 2007), and selected on yeast nitrogen base agar with 2% (w/v) glucose and CSM without tryptophan (CSM-TRP) media (Sunrise Science Products). Colonies were screened for correct assembly using Phire Plant Direct polymerase master mix (ThermoFisher Scientific). Plasmids from correct assemblies were isolated using a yeast plasmid miniprep kit (Zymo Research) and transformed into E. coli DH5-alpha using heat shock transformation with selection on LB agar with 100 μg/ml ampicillin. Plasmid was isolated from E. coli cultures and sequence confirmed using sanger sequencing.

pLOA-520 was built using TAR cloning in S. cerevisiae CEN.PK-1c. Fragments for assembly were amplified from various sources using Q5 DNA polymerase. The remainder of the process was performed in the same manner as for pLOA-519.

### Integration Methods

Fragments for integration were amplified between the TY4 sites of pLOA-519 and pLOA-520 using specific primers and Q5 DNA polymerase. Proper size was verified on an agarose gel and fragments were purified using DNA column purification. Fragments were transformed into S. cerevisiae strains using a lithium acetate transformation method (Nat. Protocols 2:31-34, 2007), and selected on yeast nitrogen base agar with 2% (w/v) glucose and CSM without uracil (CSM-URA) media (Sunrise Science Products). For transformations D556, D693, D717, and D719, 2 μg fragment DNA was used. For transformations D873 and D874, 3 μg fragment DNA was used. Plates were grown for 72 h at 30° C. prior to colony counting and downstream testing.

### Integration Efficiency

Several integrations of fragments derived from pLOA-519 and pLOA-520 were performed into various JK9-3d and CEN.PK2-1C backgrounds. To allow for constitutive expression of the pGAL1-10 promoter, knockouts in the GAL80 and MIG1 loci were generated and used for some integrations (gal80, mig1 strains). Integrations of the fragment generated from pLOA-519 into the JK9-3d and CEN.PK2-1C backgrounds resulted in >100 and 11 colonies respectively. Integrations of the same fragment into gal80, mig1 strains resulted in >100 colonies for JK9-3d and 2 colonies for CEN.PK. Integrations of the fragment generated from pLOA-520 into gal80, mig1 strains resulted in >100 colonies for JK9-3d and 8 colonies for CEN.PK2-1C.

### Production Methods

To screen for activity from integrated strains, individual colonies were picked into wells of a 96-well deepwell plate containing growth media. For testing of D556, precultures were grown in 0.3 ml per well of Delft medium (pH 6.5, 7.5 g/l ammonium sulfate, 14.4 g/l potassium phosphate monobasic, 0.5 g/l magnesium sulfate heptahydrate, 3.6 ml/l of a trace metal solution (130 g/l citric acid monohydrate, 0.574 g/l copper (II) sulfate pentahydrate, 8.07 g/l iron (III) chloride hexahydrate, 0.5 g/l boric acid, 0.333 g/l manganese (II) chloride, 0.2 g/l sodium molybdate, and 4.67 g/l zinc sulfate heptahydrate), and 1.0 ml/l of a vitamin solution (0.008 g/l biotin, 1.6 g/l calcium pantothenate, 0.008 g/l folic acid, 8 g/l myo-inositol, 1.6 g/l nicotinic acid, 0.8 g/l p-aminobenzoic acid, 1.6 g/l pyridoxal hydrochloride, 0.8 g/l riboflavin, 1.6 g/l thiamine hydrochloride, pH 10.5)) with 2% (w/v) glucose and 0.75 g/l CSM. For testing of D693, D717, D719, D873, and D874 precultures were grown in 0.3 ml per well of Delft medium with 2% (w/v) glucose, 10 g/l yeast extract, and 20 g/l peptone. Precultures were grown at 30° C. and 80% relative humidity with shaking at 900 rpm (3 mm diameter throw).

After 24 h of growth, 10 ul preculture was transferred to wells of 96 well deepwell plates containing 0.3 ml of media to start production cultures. For production, D556 was grown in Delft medium with 2% (w/v) glucose, 0.75 g/l CSM, and 3.44 mM hexanoic acid (HA). D693 was grown in Delft medium with 2% (w/v) glucose, 10 g/l yeast extract, 20 g/l peptone, and 3.44 mM HA. D717 and D719 were grown in Delft medium (pH 5.5) with 150 mM 2-ethanesulfonic acid (MES), 2% (w/v) glucose, 10 g/l yeast extract, 20 g/l peptone, and 9.08 mM butyric acid (BA). D873 and D874 were grown in Delft medium with 2% (w/v) galactose, 10 g/l yeast extract, 20 g/l peptone, and 3.44 mM HA. Sixty μl of isopropyl myristate (IPM) was added as an overlay to all wells. Cultures were grown at 30° C. and 80% relative humidity with shaking at 900 rpm (3 mm diameter throw) for 48 h (D556), 24 h (D693), or 18 h (D717, D719, D873, D874) prior to sample harvest. To quantify the production of OA and DA, the IPM layer was harvested and passed through a 0.22 micron filter plate into an HPLC collection plate (Waters). Plates were analyzed using reverse phase high-performance liquid chromatography with UV absorbance at 235 nm. Concentrations of OA and DA were calculated using a standard curve generated by synthetic OA and DA standards. An OA/DA production strain that shows consistent production characteristics that was developed internally (LSC3-134) was included as a control on all plates. This was included as a control to account for media and timing differences between D556 and D693 and possible plate-dependent differences between strains.

### Production

Integrations of the fragment generated from pLOA-519 into the JK9-3d gal80 mig1 background (D556) resulted in a maximum titer of 246 mg/L OA compared to an average of 211 mg/L OA in the control. Maximum titer from the same fragment integrated into the CEN.PK2-1C gal80 mig1 background (D693) resulted in a maximum titer of 0.37 mg/L OA compared to an average of 207 mg/L OA in the control. Integrations of the fragment generated from pLOA-520 into the JK9-3d gal80 mig1 background (D717) resulted in a maximum titer of 46 mg/L DA compared to an average of 32 mg/L for the control. None of the colonies screened from the same integration into CEN.PK2-1C gal80 mig1 (D719) produced any detectable DA while the control strain produced 33 mg/L.

### TAR Cloning Efficiency in CEN.PK and JK9-3d

pLOA-515 contains the genes Cannabis sativa acyl activating enzyme 1 (CsAAE1), Cannabis sativa tetraketide synthase (CsTKS), and Cannabis sativa olivetolic acid cyclase (sequence sourced from uniprot ID: 16WU39, 1 AA added to CsOAC from pLOA-519/pLOA-520) (CsOAC-uniprot). Genes in pLOA-515 are expressed behind the pGAL1,10 bidirectional promoter with pGAL1 driving bicistronic expression of CsTKS and CsOAC-uniprot fused with a 2A self cleaving peptide from thosea asigna virus (T2A) and pGAL10 driving expression of CsAAE1. Expression of CsOAC-uniprot and CsAAE1 are insulated by the CYC1 and GRE terminators respectively.

pLOA-515 contains a split marker gene orotidine-5′-phosphate decarboxylase from Kluyveromyces lactis (KIURA3) fused to a degradation tag for selection on uracil dropout media and yeast integrating plasmid integration into a nonfunctional KIURA3 genomic locus. KIURA3 is expressed behind its native promoter pKIURA3 and insulated by its native terminator tKIURA3. Plasmid pLOA-515 contains a CEN/ARS origin and ScTRP1 marker for maintenance and selection in S. cerevisiae and a pUC origin and Ampicillin resistance marker for maintenance and selection in E. coli.

pLOA-515 was attempted to be built using transformation associated recombination (TAR) cloning in S. cerevisiae CEN.PK-1c and S. cerevisiae JK9-3d. Fragments for assembly were amplified from various sources using Q5 DNA polymerase. Fragments from plasmid sources were digested for 1 h with FastDigest DpnI (ThermoFisher Scientific) and column purified. Equimolar concentrations of each fragment were mixed, transformed into CEN.PK2-1C MATa, his3D1, leu2-3,112, ura3-52, trp1-289 or JK9-3d MATa, leu2-3,112, ura3-52, trp1, his4 using a lithium acetate transformation method (Nat. Protocols 2:31-34, 2007), and selected on yeast nitrogen base agar with 2% (w/v) glucose and CSM without tryptophan (CSM-TRP) media (Sunrise Science Products).

Colonies were screened for correct assembly using Phire Plant Direct polymerase master mix (ThermoFisher Scientific). The overlap junction between fragments pLOA-515_1 and pLOA-515_3 was used to screen for correct assembly using gene specific primers (YO12093-ggtggtggtccagaacaattg/YO12332-gcaaatcacgtgatatagatccacg). Out of 12 colonies screened for assembly in CEN.PK, zero showed correct amplification of the overlapping region. Out of 14 colonies screened for assembly in JK9-3d, 13 showed correct amplification of the overlapping region.

Two colonies from each transformation were selected, grown overnight in CSM-Trp media, and had plasmid isolated using a yeast plasmid miniprep kit (Zymo Research). These plasmids were transformed into E. coli DH5-alpha using heat shock transformation with selection on LB agar with 100 μg/ml ampicillin. Plasmid was then amplified and isolated from E. coli cultures and sequenced using sanger sequencing (Genewiz). The two colonies sequenced from TAR cloning in CEN.PK (C4 and C12) showed large fragments that had been removed from the expected gene cassette. C4 is missing CsAAE1 and C12 is missing CsAAE1 and a portion of CsTKS. Neither portion of the cassette that is missing from these clones aligns with the overlapping regions in TAR cloning and the missing regions differ from one another, suggesting that these portions are being excised post fragment assembly. The missing regions include the binding site for one of the primers used during colony screening, explaining why these did not amplify. Both clones assembled in JK9-3d that were sequenced (C₁ and C₂) contained full gene cassettes without mutations.

### Example 6A

Strain: LSC300004
Genotype: gal80\hat{ }:: pScTEF1>PkHIS4<tScGAL80
Parent strain: LSC300002
Genotype of parent strain: pGal10-CsAEE1-tCyc1-pGal1-TKST2AOAC-tCyc1::leu2-3, 112_pGal10-CsAEE1-tCyc1-pGal1-TKST2AOAC-tCyc1:: ura3-52_pGal10-CsAEE1-tCyc1-pGal1-TKST2AOAC-tCyc1:: trp1_his4_pGal10-HMGK2R-tADH1-pGal1-IDI1-tCyc1-Kan MX::YORWΔ22

### Fermentation Process Summary:

### Seed Train:

A shake flask containing 50 mL YPD (10 g/L yeast extract, 20 g/L peptones and 20 g/L glucose) was inoculated with freshly streaked LSC300004. Strain grew at 30 C for 24 hours to an OD600 of 8. 40 ml of this culture was used to inoculate the fermentation tank.

### Media:

Seed Media: YP with 20 g/L glucose
Batch media:
1× YP+55 g/L glucose+500 mg/L histidine+12 mg/L myo-inositol+12 mg/L thiamin hydrochloride+12 mg/L pyridoxal hydrochloride+12 mg/L nicotinic acid+12 mg/L calcium pantothenate+0.6 mg/L biotin+12 mg/L p-aminobenzoic acid+0.15 g/L EDTA+7.8 mg/L CuSO4-5H2O+0.0512 g/L FeSO4-7H2O+0.0032 g/L MnCl2+4.77 mg/L Na2MoO4+0.102 g/L ZnSO4-7H2O+0.0086 g/L CoCl2-6H2O+0.0384 g/L CaCl2-2H2O+5.5 g/L KH2PO4+2.9 g/L MgSO4-7H2O+45.1 g/L (NH4)2SO4

### Growth Media:

600 g/L glucose+500 mg/L histidine

### Production Media:

650 g/L glucose+10 g/L hexanoic acid

### Base (for pH Control)

5M NH₄OH

### Galactose Addition

4 g galactose was added to tank at 24, 48 and 120 hours, respectively.

### Overlay

100 mL isopropyl myristate (25% V/VO) was added to tank at 24 hours. Additionally, 10 mL (2.5% V/Vo) isopropyl myristate was added to tank at 48 and 120 hours, respectively.

### Antifoam

Struktol SB2121 (0.1 mL/L) at the beginning of the run
Struktol SB509 (0.5 mL/day)

### Fermentation Run Condition:

We used pulse feeding for both growth phase and production phase during the run. Fermentation batch was inoculated with 40 ml of inoculum. Feeding was triggered at the end of batch phase when batch glucose was completely exhausted and pO2 was increased by 20% (or more). Feed media (growth or production media) was delivered in pulses. Each pulse delivered 2 g glucose/starting batch volume with maximum feed rate not exceeding 20 g glucose/L/hr. pH was maintained at 6 throughout the run. Temperature of fermenter was maintained at 30C. Air flow rate was maintained at 1.25 L/min. Agitation was 800 rpm.

### Summary of Metrics:

Maximum Olivetolic acid titer: 3.48 g/L
Maximum Olivetol: 0.9 g/L
Total product titer: 4.36 g/L
Titer/time at 119 hours: 0.86 g/L/day
Cumulative yield of olivetolic acid/HA consumed: 0.49 mol/mol.
Cumulative yield of olivetol/HA consumed: 0.15 mol/mol

### Example 6B

Compared to example 6A, we used a different strain and did not add any galactose in this run.
Strain: LSC300013
Genotype: mig1\hat{ }:: HygR
Parent strain: LSC300005 (sister clone of LSC300004)
Genotype of parent strain: See example 6A

### Fermentation Process Summary:

### Seed Train:

A shake flask containing 50 mL YPD with 20 g/L glucose was inoculated with freshly streaked LSC300013. Strain grew at 30 C for 24 hours to an OD600 of 8. 40 mL of this culture was used to inoculate the fermentation tank.

### Media:

Seed Media: YP with 20 g/L glucose

### Batch Media:

1× YP+55 g/L glucose+500 mg/L histidine+12 mg/L myo-inositol+12 mg/L thiamin hydrochloride+12 mg/L pyridoxal hydrochloride+12 mg/L nicotinic acid+12 mg/L calcium pantothenate+0.6 mg/L biotin+12 mg/L p-aminobenzoic acid+0.15 g/L EDTA+7.8 mg/L CuSO4-5H2O+0.0512 g/L FeSO4-7H2O+0.0032 g/L MnCl2+4.77 mg/L Na2MoO4+0.102 g/L ZnSO4-7H2O+0.0086 g/L CoCl2-6H2O+0.0384 g/L CaCl2-2H2O+5.5 g/L KH2PO4+2.9 g/L MgSO4-7H2O+45.1 g/L (NH4)2SO4

### Growth Media:

600 g/L glucose+500 mg/L histidine

### Production Media:

650 g/L glucose+10 g/L hexanoic acid

### Base (for pH Control)

5M NH₄OH

### Overlay:

100 mL isopropyl myristate (25% V/VO) was added to tank at 24 hours. Additionally, 10 mL (2.5% V/Vo) isopropyl myristate was added to tank at 48 and 120 hours, respectively.

### Antifoam

Struktol SB2121 (0.1 mL/L) at the beginning of the run
Struktol SB509 (0.5 mL/day)

### Fermentation Run Condition:

We used pulse feeding for both growth phase and production phase during the run. Fermentation batch was inoculated with 40 ml of inoculum. Feeding was triggered at the end of batch phase when batch glucose was completely exhausted and p02 was increased by 20% (or more). Feed media (growth or production media) was delivered in pulses. Each pulse delivered 2 g glucose/starting batch volume with maximum feed rate not exceeding 20 g glucose/L/hr. pH was maintained at 6 throughout the run. Temperature of fermenter was maintained at 30C. Air flow rate was maintained at 1.25 L/min. Agitation was 800 rpm.

### Summary of Metrics:

Maximum Olivetolic acid titer: 2.92 g/L
Maximum Olivetol: 0.65 g/L
Total product titer: 3.53 g/L
Titer/time at 119 hours: 0.71 g/L/day
Cumulative yield of olivetolic acid/HA consumed: 0.49 mol/mol
Cumulative yield of olivetol/HA consumed: 0.10 mol/mol

### Example 6C

Compared to example 6B, we used different media and maximum feed rate was higher.
Strain: LSC300013
Genotype: mig1\hat{ }:: HygR
Parent strain: LSC300005 (sister clone of LSC300004)
Genotype of parent strain: See example 6A

### Fermentation Process Summary:

### Seed Train:

A shake flask containing 50 mL YPD with 20 g/L glucose was inoculated with freshly streaked LSC300013. Strain grew at 30 C for 24 hours to an OD600 of 4. 40 ml of this culture was used to inoculate the fermentation tank.

### Media:

Seed Media: YP with 20 g/L Glucose
Batch media:
1× YP+55 g/L glucose+500 mg/L histidine+12 mg/L myo-inositol+12 mg/L thiamin hydrochloride+12 mg/L pyridoxal hydrochloride+12 mg/L nicotinic acid+12 mg/L calcium pantothenate+0.6 mg/L biotin+12 mg/L p-aminobenzoic acid+0.15 g/L EDTA+7.8 mg/L CuSO4-5H2O+0.0512 g/L FeSO4-7H2O+0.0032 g/L MnCl2+4.77 mg/L Na2MoO4+0.102 g/L ZnSO4-7H2O+0.0086 g/L CoCl2-6H2O+0.0384 g/L CaCl2-2H2O+5.5 g/L KH2PO4+2.9 g/L MgSO4-7H2O+45.1 g/L (NH4)2SO4

### Production Media:

650 g/L glucose+438 mg/L citric acid monohydrate+2 mg/L H3BO3+1.3 mg/L CuSO4-5H2O+22.4 mg/L FeCl3-6H2O+1.33 mg/L MnCl2+0.8 mg/L Na2MoO4+10.8 mg/L ZnSO4-7H2O+12 mg/L myo-inositol+12 mg/L thiamin hydrochloride+12 mg/L pyridoxal hydrochloride+12 mg/L nicotinic acid+12 mg/L calcium pantothenate+12 mg/L biotin+12 mg/L p-aminobenzoic acid+12 mg/L folic acid+12 mg/L riboflavin+2.5 g/L KH2PO4+1 g/L MgSO4-7H2O+20 g/L (NH4)2SO4+17.8 g/L sodium hexanoate
Base (for pH Control): 5M NH₄OH

### Overlay:

100 mL isopropyl myristate (25% V/VO) was added to tank at 24 hours. Additionally, 10 mL (2.5% V/Vo) isopropyl myristate was added to tank at 96, 120 and 144 hours, respectively.

### Antifoam

Struktol SB2121 (0.1 mL/L) at the beginning of the run
Struktol SB509 (0.5 mL/day)

### Fermentation Run Condition:

We used pulse feeding for both growth phase and production phase during the run. Fermentation batch was inoculated with 40 ml of inoculum. Feeding was triggered at the end of batch phase when batch glucose was completely exhausted and pO2 was increased by 20% (or more). Feed media (growth or production media) was delivered in pulses. Each pulse delivered 2 g glucose/starting batch volume with maximum feed rate not exceeding 40 g glucose/L/hr. pH was maintained at 6 throughout the run. Temperature of fermenter was maintained at 30C. Air flow rate was maintained at 1.25 L/min. Agitation was 800 rpm. This fermentation works at pH range of 5-6. A pH 5.0 may work better. Pulse rate of 1.7 g/L/pulse, with maximum feed rate of 10 g/L/hr may work better.

### Summary of Metrics:

Maximum Olivetolic acid titer: 6.07 g/L
Maximum Olivetol: 2.03 g/L
Total product titer: 8 g/L
Titer/time at 119 hours: 1.5 g/L/day
Cumulative yield of olivetolic acid/HA consumed: 0.93 g/g
Cumulative yield of olivetol/HA consumed: 0.27 g/g

### Example 6D

Strain: LSC3-134
   Genotype: gal80\hat{ }:: (loxHIS4)/his4\hat{ }/mig1\hat{ }:: (loxPkHIS4)
Parent strain: LSC300002
Genotype of parent strain: pGal10-CsAEE1-tCyc1-pGal1-TKST2AOAC-tCyc1::leu2-3, 112 pGal10-CsAEE1-tCyc1-pGal1-TKST2AOAC-tCyc1:: ura3-52_pGal10-CsAEE1-tCyc1-pGal1-TKST2AOAC-tCyc1:: trp1_his4_pGal10-HMGK2R-tADH1-pGal1-IDI1-tCyc1-Kan MX::YORWΔ22

### Fermentation Process Summary:

### Seed Train:

A shake flask containing 50 mL YPD (10 g/L yeast extract, 20 g/L peptones and 20 g/L glucose) was inoculated with freshly streaked LSC3-134. Strain grew at 30 C for 24 hours to an OD600 of 4. 17 mL of this culture was used to inoculate the fermentation tank (3.5% of initial tank volume).

### Media:

### Seed Media: YPD (10 g/L Yeast Extract+20 g/L Peptones+20 g/L Glucose)

### Batch Media:

10 g/L yeast extract+20 g/L peptones+20 g/L glucose+500 mg/L histidine+12 mg/L myo-inositol+12 mg/L thiamin hydrochloride+12 mg/L pyridoxal hydrochloride+12 mg/L nicotinic acid+12 mg/L calcium pantothenate+0.6 mg/L biotin+12 mg/L p-aminobenzoic acid+0.15 g/L EDTA+7.8 mg/L CuSO4-5H2O+0.0512 g/L FeSO4-7H2O+0.0032 g/L MnCl2+4.77 mg/L Na2MoO4+0.102 g/L ZnSO4-7H2O+0.0086 g/L CoCl2-6H2O+0.0384 g/L CaCl2-2H2O+5.5 g/L KH2PO4+2.9 g/L MgSO4-7H2O+45.1 g/L (NH4)2SO4

### Production Media:

650 g/L glucose+438 mg/L citric acid monohydrate+2 mg/L H3BO3+1.3 mg/L CuSO4-5H2O+22.4 mg/L FeCl3-6H2O+1.33 mg/L MnCl2+0.8 mg/L Na2MoO4+10.8 mg/L ZnSO4-7H2O+12 mg/L myo-inositol+12 mg/L thiamin hydrochloride+12 mg/L pyridoxal hydrochloride+12 mg/L nicotinic acid+12 mg/L calcium pantothenate+12 mg/L biotin+12 mg/L p-aminobenzoic acid+12 mg/L folic acid+12 mg/L riboflavin+2.5 g/L KH2PO4+1 g/L MgSO4-7H2O+20 g/L (NH4)2SO4+36 g/L sodium hexanoate
Base (for pH Control): 5M NH₄OH

### Overlay:

182 mL isopropyl myristate (40% V/VO) was added to tank at 24 hours. Stir rate was reduced to 500 rpm before addition of IPM at 24 hours and was increased to 800 rpm at around 48 hours. This step was performed to eliminate the risk of foaming after IPM addition. 30% to 50% positive pO2 was maintained between 24 and 48 hours runs time. 1.6%) V/V) antifoam was added to IPM before addition to tank.
Antifoam: Struktol SB2121 (0.1 mL/L) at the beginning of the run

### Fermentation Run Condition:

We used pulse feeding for both growth phase and production phase during the run. Fermentation batch was inoculated with 17 mL of inoculum. Feeding was triggered at the end of batch phase when batch glucose was completely exhausted and p02 was increased by 10% (or more). Feed media (production media) was delivered in pulses. Each pulse delivered 1.7 g glucose/starting batch volume with maximum feed rate not exceeding 10 g glucose/L/hr. pH was maintained at 5.5 throughout the run. Temperature of fermenter was maintained at 30C. Air flow rate was maintained at 1.25 L/min. Agitation was 800 rpm. Median oxygen uptake rate (OUR) was 60-80 mmoles/L/hr. A maximum OUR of 100-110 moles/L/hr was achieved during the process.

### Summary of Metrics:

Maximum Olivetolic acid titer: 6.9510.22
Maximum Olivetol: 2.68+0.24
Titer/time at 96 hours: 2.2 g/L/day (OA+O)
Cumulative yield of olivetolic acid/HA consumed: 1.4 g/g
Cumulative yield of olivetol/HA consumed: 0.56 g/g

### Effect of pH on Process Metrics

Under the test conditions, optimum pH for the process was 5.5±0.3.

### Effect of Temperature on Process Metrics

Under the test conditions, optimum temperature for the process was 30±2

Optimum time to add IPM: Under the test conditions, optimum time to add IPM was between 12 and 36 hours post inoculation.

Effect of sodium hexanoate/glucose ratio in feed: In a series of experiments, we tested sensitivity of metrics (titer and productivity) to the ratio of sodium hexanoate to glucose in feed. Under the test conditions, maximum olivetol equivalent titer was achieved when sodium hexanoate/glucose in feed ratio was in the range of 20 to 28 g sodium hexanoate/500 g glucose. Under the test conditions, maximum productivity was achieved at a range of 23 to 28 g sodium hexanoate/500 g glucose in feed.

Effect of Oxygen transfer rate on metrics: Under the test conditions, the optimum median OTR for the process is 60-80 mmoles/L/hr and a maximum OUR of 100-110 mmoles/L/hr is achieved in the process.

Pulse metric parameters: Under the test conditions, optimum pulse parameters for the process was 1.7 g glucose/L initial tank volume/pulse with a maximum feed rate of 10 g/L of initial tank volume/hr.

Effect of overlay: Isopropyl myristate is used as an overlay in our process. Under the test conditions, the optimum IPM loading for our process at pH 5.5 is 26% of total tank volume or 40% of initial tank volume. There was a clear negative effect when no IPM was used.

Effect of batch glucose concentration: Under the test conditions, the optimum batch glucose concentration for our process was 10-20 g/L.

Seed train condition: Under the test conditions, the optimum seed train condition was to inoculate an initial flask containing YPD (10 g/L yeast extract, 20 g/L peptones and 20 g/L glucose) with 1 mL seed vial. In certain instances, tanks are contemplated to be inoculated with 2% inoculum and will run as batch tanks with pH control (pH set at 5.5). In certain instances, the production tank will be inoculated with 3.5% inoculum from the last seed train stage.
Batch media composition for seed tanks: 1× YP+55 g/L glucose+500 mg/L histidine+12 mg/L myo-inositol+12 mg/L thiamin hydrochloride+12 mg/L pyridoxal hydrochloride+12 mg/L nicotinic acid+12 mg/L calcium pantothenate+0.6 mg/L biotin+12 mg/L p-aminobenzoic acid+0.15 g/L EDTA+7.8 mg/L CuSO4-5H2O+0.0512 g/L FeSO4-7H2O+0.0032 g/L MnCl2+4.77 mg/L Na2MoO4+0.102 g/L ZnSO4-7H2O+0.0086 g/L CoCl2-6H2O+0.0384 g/L CaCl₂)-2H2O+5.5 g/L KH2PO4+2.9 g/L MgSO4-7H2O+45.1 g/L (NH4)2SO4
Batch media composition for production tanks: 1× YP+17-20 g/L glucose+500 mg/L histidine+12 mg/L myo-inositol+12 mg/L thiamin hydrochloride+12 mg/L pyridoxal hydrochloride+12 mg/L nicotinic acid+12 mg/L calcium pantothenate+0.6 mg/L biotin+12 mg/L p-aminobenzoic acid+0.15 g/L EDTA+7.8 mg/L CuSO4-5H2O+0.0512 g/L FeSO4-7H2O+0.0032 g/L MnCl2+4.77 mg/L Na2MoO4+0.102 g/L ZnSO4-7H2O+0.0086 g/L CoCl2-6H2O+0.0384 g/L CaCl2-2H2O+5.5 g/L KH2PO4+2.9 g/L MgSO4-7H2O+45.1 g/L (NH4)2SO4
Production Media (for production tank): 650 g/L glucose+438 mg/L citric acid monohydrate+2 mg/L H3BO3+1.3 mg/L CuSO4-5H2O+22.4 mg/L FeCl3-6H2O+1.33 mg/L MnCl2+0.8 mg/L Na2MoO4+10.8 mg/L ZnSO4-7H2O+12 mg/L myo-inositol+12 mg/L thiamin hydrochloride+12 mg/L pyridoxal hydrochloride+12 mg/L nicotinic acid+12 mg/L calcium pantothenate+12 mg/L biotin+12 mg/L p-aminobenzoic acid+12 mg/L folic acid+12 mg/L riboflavin+2.5 g/L KH2PO4+1 g/L MgSO4-7H2O+20 g/L (NH4)2SO4+36 g/L sodium hexanoate
Base (for pH Control): 5-10 M NH₄OH

### Example 7A Polypropylene Glycol (PPG) as Immiscible Overlay

Strain: LSC3-134
   Genotype: gal80\hat{ }:: (loxHIS4)/his4\hat{ }/mig1\hat{ }:: (loxPkHIS4)
Parent strain: LSC300002
Genotype of parent strain: pGal10-CsAEE1-tCyc1-pGal1-TKST2AOAC-tCyc1::leu2-3, 112_pGal10-CsAEE1-tCyc1-pGal1-TKST2AOAC-tCyc1:: ura3-52_pGal10-CsAEE1-tCyc1-pGal1-TKST2AOAC-tCyc1:: trp1_his4_pGal10-HMGK2R-tADH1-pGal1-IDI1-tCyc1-Kan MX::YORWΔ22

### Fermentation Process Summary:

### Seed Train:

A shake flask containing 50 mL YPD (10 g/L yeast extract, 20 g/L peptones and 20 g/L glucose) was inoculated with freshly streaked LSC3-134. Strain grew at 30 C for 24 hours to an OD600 of 8-10. This culture was used to inoculate the fermentation tank (inoculum: 3.5% of initial tank volume).

### Media:

### Seed Media: YPD (10 g/L Yeast Extract+20 g/L Peptones+20 g/L Glucose)

### Batch Media:

10 g/L yeast extract+20 g/L peptones+20 g/L glucose+500 mg/L histidine+12 mg/L myo-inositol+12 mg/L thiamin hydrochloride+12 mg/L pyridoxal hydrochloride+12 mg/L nicotinic acid+12 mg/L calcium pantothenate+0.6 mg/L biotin+12 mg/L p-aminobenzoic acid+0.15 g/L EDTA+7.8 mg/L CuSO4-5H2O+0.0512 g/L FeSO4-7H2O+0.0032 g/L MnCl2+4.77 mg/L Na2MoO4+0.102 g/L ZnSO4-7H2O+0.0086 g/L CoCl2-6H2O+0.0384 g/L CaCl2-2H2O+5.5 g/L KH2PO4+2.9 g/L MgSO4-7H2O+45.1 g/L (NH4)2SO4

### Production Media:

650 g/L glucose+438 mg/L citric acid monohydrate+2 mg/L H3BO3+1.3 mg/L CuSO4-5H2O+22.4 mg/L FeCl3-6H2O+1.33 mg/L MnCl2+0.8 mg/L Na2MoO4+10.8 mg/L ZnSO4-7H2O+12 mg/L myo-inositol+12 mg/L thiamin hydrochloride+12 mg/L pyridoxal hydrochloride+12 mg/L nicotinic acid+12 mg/L calcium pantothenate+12 mg/L biotin+12 mg/L p-aminobenzoic acid+12 mg/L folic acid+12 mg/L riboflavin+2.5 g/L KH2PO4+1 g/L MgSO4-7H2O+20 g/L (NH4)2SO4+36 g/L sodium hexanoate
Base (for pH Control): 5M NH₄OH

### Overlay:

170 mL polypropylene glycol with an average molecular weight of 1200 was added to 400 mL batch media (42.5% V/VO) and autoclaved in tank.

### Fermentation Run Condition:

We used pulse feeding during the run. Fermentation batch was inoculated with 3.5% (V/Vo) inoculum. Feeding was triggered at the end of batch phase when batch glucose was completely exhausted and p02 was increased by 10% (or more). Feed media (production media) was delivered in pulses. Each pulse delivered 1.7 g glucose/starting batch volume with maximum feed rate not exceeding 10 g glucose/L/hr. pH was maintained at 5.5 throughout the run. Temperature of fermenter was maintained at 30C. Air flow rate was maintained at 1.25 L/min. Agitation was set at 1200 rpm.

### Summary of Metrics:

Maximum olivetolic acid (OA) titer with PPG as overlay was 13.3 g/L
OA fermentation productivity (titer/time) at 136 hour was 2.4 g/L/day.
Olivetolic Acid/Olivetol ratio in PPG was approximately 1.5 times higher than the ratio of OA/O in IPM.

### Example 7B Fermentation Using Polypropylene Glycol of Various Molecular Weights

### Strain: LSC3-433

Genotype: JK9-3d MATa gal80Δ::lox72 mig14::lox72 TRP1::pLOA-006 URA3::pLOA-007 LEU2::pLOA-019 HIS4: pLOA-425

### Fermentation Process Summary:

### Seed Train:

A shake flask containing 50 mL YPD (10 g/L yeast extract, 20 g/L peptones and 20 g/L glucose) was inoculated with freshly streaked LSC3-433. Strain grew at 30 C for 24 hours to an OD600 of 8-10. This culture was used to inoculate the fermentation tank (inoculum: 3.5% of initial tank volume).

### Media:

Seed Media: YPD (10 g/L yeast extract+20 g/L peptones+20 g/L glucose) Batch media:
10 g/L yeast extract+20 g/L peptones+20 g/L glucose+500 mg/L histidine+12 mg/L myo-inositol+12 mg/L thiamin hydrochloride+12 mg/L pyridoxal hydrochloride+12 mg/L nicotinic acid+12 mg/L calcium pantothenate+0.6 mg/L biotin+12 mg/L p-aminobenzoic acid+0.15 g/L EDTA+7.8 mg/L CuSO4-5H2O+0.0512 g/L FeSO4-7H2O+0.0032 g/L MnCl2+4.77 mg/L Na2MoO4+0.102 g/L ZnSO4-7H2O+0.0086 g/L CoCl2-6H2O+0.0384 g/L CaCl2-2H2O+5.5 g/L KH2PO4+2.9 g/L MgSO4-7H2O+45.1 g/L (NH4)2SO4

### Production Media:

650 g/L glucose+438 mg/L citric acid monohydrate+2 mg/L H3BO3+1.3 mg/L CuSO4-5H2O+22.4 mg/L FeCl3-6H2O+1.33 mg/L MnCl2+0.8 mg/L Na2MoO4+10.8 mg/L ZnSO4-7H2O+12 mg/L myo-inositol+12 mg/L thiamin hydrochloride+12 mg/L pyridoxal hydrochloride+12 mg/L nicotinic acid+12 mg/L calcium pantothenate+12 mg/L biotin+12 mg/L p-aminobenzoic acid+12 mg/L folic acid+12 mg/L riboflavin+2.5 g/L KH2PO4+1 g/L MgSO4-7H2O+20 g/L (NH4)2SO4+36 g/L sodium hexanoate
Base (for pH Control): 5M NH₄OH

### Overlay:

100 mL polypropylene glycol with an average molecular weight of 1200 or 1500 or 4000 (PPG1200, PPG1500, or PPG4000) was added to 400 mL batch media (25% V/V0) and autoclaved in tank.

### Fermentation Run Condition:

We used pulse feeding during the run. Fermentation batch was inoculated with 3.5% (V/Vo) inoculum. Feeding was triggered at the end of batch phase when batch glucose was completely exhausted and p02 was increased by 10% (or more). Feed media (production media) was delivered in pulses. Each pulse delivered 1.7 g glucose/starting batch volume with maximum feed rate not exceeding 10 g glucose/L/hr. pH was maintained at 5.5 throughout the run. Temperature of fermenter was maintained at 30C. Air flow rate was maintained at 1.25 L/min. Agitation was set at 1200 rpm.

### Summary of Metrics:

Observed maximum olivetolic acid (OA) titer was in the range of 11-13.5 g/L at 150 hours and all three PPG types of different average molecular weights worked equally well as immiscible overlays in this example.

### Example 7C Effect of the Polypropylene Glycol Loading Amount on Fermentation Performance

Strain: LSC3-433

### Fermentation Process Summary:

Fermentation run conditions and media are the same as in example 7B.

### Overlay:

Polypropylene glycol with an average molecular weight of 1200 was added to tanks at the beginning of the runs. The percentage of PPG was calculated relative to the full tank volume and varied between 2.5% and 17.5%.
   Summary of results:
Under the test condition, the optimum range for our process was with PPG loading in the range of 5-10% relative to tank full volume. Under the test condition, the optimum oxygen transfer rate for our process ranged between 60 and 100 mmoles/L/hr.

### Example 7D: Feeding Optimization

Strain: LSC3-433

### Fermentation Process Summary:

Fermentation run conditions and seed and batch media are the same as in example 7C. The ratio of sodium hexanoate to glucose in the feed media varied between different runs. All runs delivered the same amount of sodium hexanoate/L/pulse (0.094 g/L initial tank volume/pulse), but the glucose feed rates varied between different runs. Polypropylene glycol with an average molecular weight of 1200 was added to tanks at the beginning of the runs. The percentage of PPG relative to the full tank volume was 5%.

Summary of results:
Under the test condition, an increase in sodium hexanoate/glucose ratio from baseline value of 0.055 to 0.08 while maintaining sodium hexanoate/L/pulse at 0.094 g/L (based on initial tank volume)/pulse, resulted in the highest productivity and OA titer in these experiments.
Maximum OA productivity observed (at 120 hours): 2.7 g/L/day.
Maximum OA titer observed (at 120 hours): 13.5 g/L.

### Example 8A

Strain: LSC3-134
Genotype: JK9-3d MATa gal80Δ::lox72 mig1Δ::lox66-PkHIS4-lox71 TRP1::pLOA-006 URA3::pLOA-007 LEU2::pLOA-019

### Fermentation Process Summary:

### Seed Train:

A shake flask containing 50 mL YPD with 20 g/L glucose was inoculated with a seed vial containing LSC3-134. Strain grew at 30 C for 24 hours to an OD600 of 3-4. 15 mL of this culture was used to inoculate the fermentation tank.

### Media:

Seed Media: YP with 20 g/L Glucose

### Batch Media:

10 g/L yeast extract+20 g/L peptones+20 g/L glucose+500 mg/L histidine+12 mg/L myo-inositol+12 mg/L thiamin hydrochloride+12 mg/L pyridoxal hydrochloride+12 mg/L nicotinic acid+12 mg/L calcium pantothenate+0.6 mg/L biotin+12 mg/L p-aminobenzoic acid+0.15 g/L EDTA+7.8 mg/L CuSO4-5H2O+0.0512 g/L FeSO4-7H2O+0.0032 g/L MnCl2+4.77 mg/L Na2MoO4+0.102 g/L ZnSO4-7H2O+0.0086 g/L CoCl2-6H2O+0.0384 g/L CaCl2-2H2O+5.5 g/L KH2PO4+2.9 g/L MgSO4-7H2O+45.1 g/L (NH4)2SO4

### Production Media:

650 g/L glucose+438 mg/L citric acid monohydrate+2 mg/L H3BO3+1.3 mg/L CuSO4-5H2O+22.4 mg/L FeCl3-6H2O+1.33 mg/L MnCl2+0.8 mg/L Na2MoO4+10.8 mg/L ZnSO4-7H2O+12 mg/L myo-inositol+12 mg/L thiamin hydrochloride+12 mg/L pyridoxal hydrochloride+12 mg/L nicotinic acid+12 mg/L calcium pantothenate+12 mg/L biotin+12 mg/L p-aminobenzoic acid+12 mg/L folic acid+12 mg/L riboflavin+2.5 g/L KH2PO4+1 g/L MgSO4-7H2O+20 g/L (NH4)2SO4+20 g/L sodium butyrate

### Base (for pH Control)

5M NH₄OH

### Overlay

170 mL isopropyl myristate (40% V/VO) was added to tank at 24 hours.
Antifoam Struktol SB2121 (0.1 mL/L) at the beginning of the run

### Fermentation Run Condition:

We used pulse feeding during the run. Fermentation batch was inoculated with 15 mL of inoculum. Feeding was triggered at the end of batch phase when batch glucose was completely exhausted and pO2 was increased by 10% (or more). Feed media (growth or production media) was delivered in pulses. Each pulse delivered 1.7 g glucose/starting batch volume with maximum feed rate not exceeding 10 g glucose/L/hr. pH was maintained at 5.5 throughout the run. Temperature of fermenter was maintained at 30C. Air flow rate was maintained at 1.25 L/min. Agitation was 800 rpm.

### Summary of Metrics:

Maximum Divarinic acid titer: 2.5 g/L
Maximum Divarin: 2.5 g/L
Total product titer: 5.0 g/L (D+DA)
Titer/time: 0.74 g Divarin equivalent/L/day

We also evaluated the effect of sodium butyrate concentration in feed in a set of three runs (10, 14.3 and 20 g/L sodium butyrate in feed, all other nutrients remained the same as stated above) and we observed that titer and productivity increased as we increased sodium butyrate concentration in feed. Under the test condition, the highest titer (and productivity) was observed in the run with 20 g/L sodium butyrate in feed.

### Example 8B

Strain: LSC3-134, LSC3-426, LCS3-520
Genotype (LSC3-134): JK9-3d MATa gal80Δ::lox72 mig14::lox66-PkHIS4-lox71 TRP1::pLOA-006 URA3::pLOA-007 LEU2::pLOA-019
Genotype (LSC3-426): JK9-3d MATa gal80Δ::lox72 mig14::lox72 TRP1::pLOA-006 URA3::pLOA-007 LEU2::pLOA-019 HIS4::pLOA-419
Genotype (LCS3-520): JK9-3d MATa gal80Δ::lox72 mig14::lox72 trp14: TRP1 (S288c) leu2Δ::LEU2 (S288c) his4Δ::HIS4 (CEN.PK2-1Ca) ura3Δ::lox66-HygR-lox71-(KIURA3-K28*-deg) KIURA3-K28*-deg::pLOA-516

### Fermentation Process Summary:

### Seed Train:

A shake flask containing 50 mL YPD with 20 g/L glucose was inoculated with a seed vial containing LSC3-134, LSC3-426 or LSC3-520. Strains grew at 30 C for 24 hours to an OD600 of 10-12. 3.5 mL of this culture was used to inoculate the fermentation tank.

### Media:

Seed Media: YP with 20 g/L Glucose

### Batch Media:

10 g/L yeast extract+20 g/L peptones+20 g/L glucose+500 mg/L histidine+12 mg/L myo-inositol+12 mg/L thiamin hydrochloride+12 mg/L pyridoxal hydrochloride+12 mg/L nicotinic acid+12 mg/L calcium pantothenate+0.6 mg/L biotin+12 mg/L p-aminobenzoic acid+0.15 g/L EDTA+7.8 mg/L CuSO4-5H2O+0.0512 g/L FeSO4-7H2O+0.0032 g/L MnCl2+4.77 mg/L Na2MoO4+0.102 g/L ZnSO4-7H2O+0.0086 g/L CoCl2-6H2O+0.0384 g/L CaCl2-2H2O+5.5 g/L KH2PO4+2.9 g/L MgSO4-7H2O+45.1 g/L (NH4)2SO4

### Production Media:

500.5 g/L glucose+337.26 mg/L citric acid monohydrate+1.54 mg/L H3BO3+1 mg/L CuSO4-5H2O+17.25 mg/L FeCl3-6H2O+1 mg/L MnCl2+0.62 mg/L Na2MoO4+8.32 mg/L ZnSO4-7H2O+9.24 mg/L myo-inositol+9.24 mg/L thiamin hydrochloride+9.24 mg/L pyridoxal hydrochloride+9.24 mg/L nicotinic acid+9.24 mg/L calcium pantothenate+9.24 mg/L biotin+9.24 mg/L p-aminobenzoic acid+9.24 mg/L folic acid+9.24 mg/L riboflavin+1.93 g/L KH2PO4+0.77 g/L MgSO4-7H2O+15.4 g/L (NH4)2SO4+11.03 g/L sodium butyrate

### Base (for pH Control)

5M NH₄OH

### Overlay

38 mL isopropyl myristate (38% V/VO) was added to tank at 24 hours.
Antifoam Struktol SB2121 (0.1 mL/L) at the beginning of the run

### Fermentation Run Condition:

We used pulse feeding during the run. Fermentation batch was inoculated with 3.5 ml of inoculum (3.5% V/VO). Feeding was triggered at the end of batch phase when batch glucose was completely exhausted and pO2 was increased by 10% (or more). Feed media (production media) was delivered in pulses. Each pulse delivered 1.7 g glucose/starting batch volume with maximum feed rate not exceeding 10 g glucose/L/hr. PH was maintained at 5.5 throughout the run. Temperature of fermenter was maintained at 30C. Air flow rate was maintained at 100 mL/min. Agitation was 2000 rpm.

Summary of metrics:

### LSC3-134:

Maximum Divarinic acid: 2.4 g/L
Maximum Divarin: 1.7 g/L
Titer/time: 0.7 g Divarin equivalent/L/day
(Divarinic acid) Titer/time: 0.47 g/L/day

### LSC3-426:

Maximum Divarinic acid: 3.3 g/L
Maximum Divarin: 2.3 g/L
Titer/time: 1.09 g Divarin equivalent/L/day
(Divarinic acid) Titer/time: 0.78 g/L/day

### LSC3-520:

Maximum Divarinic acid: 4.1 g/L
Maximum Divarin: 1.6 g/L
Titer/time: 1.1 g Divarin equivalent/L/day
(Divarinic acid) Titer/time: 1 g/L/day

### Example 8C

Strain: LSC3-426
Genotype (LSC3-426): JK9-3d MATa gal80Δ::lox72 mig14::lox72 TRP1::pLOA-006 URA3::pLOA-007 LEU2::pLOA-019 HIS4::pLOA-419

### Fermentation Process Summary:

### Seed Train:

A shake flask containing 50 mL YPD with 20 g/L glucose was inoculated with a seed vial containing LSC3-426. Strains grew at 30 C for 24 hours to an OD600 of 10-12. 3.5 ml of this culture was used to inoculate the fermentation tank.

### Media:

Seed Media: YP with 20 g/L Glucose

### Batch Media:

10 g/L yeast extract+20 g/L peptones+20 g/L glucose+500 mg/L histidine+12 mg/L myo-inositol+12 mg/L thiamin hydrochloride+12 mg/L pyridoxal hydrochloride+12 mg/L nicotinic acid+12 mg/L calcium pantothenate+0.6 mg/L biotin+12 mg/L p-aminobenzoic acid+0.15 g/L EDTA+7.8 mg/L CuSO4-5H2O+0.0512 g/L FeSO4-7H2O+0.0032 g/L MnCl2+4.77 mg/L Na2MoO4+0.102 g/L ZnSO4-7H2O+0.0086 g/L CoCl2-6H2O+0.0384 g/L CaCl₂)-2H2O+5.5 g/L KH2PO4+2.9 g/L MgSO4-7H2O+45.1 g/L (NH4)2SO4

### Production Media:

500.5 g/L glucose+337.26 mg/L citric acid monohydrate+1.54 mg/L H3BO3+1 mg/L CuSO4-5H2O+17.25 mg/L FeCl3-6H2O+1 mg/L MnCl2+0.62 mg/L Na2MoO4+8.32 mg/L ZnSO4-7H2O+9.24 mg/L myo-inositol+9.24 mg/L thiamin hydrochloride+9.24 mg/L pyridoxal hydrochloride+9.24 mg/L nicotinic acid+9.24 mg/L calcium pantothenate+9.24 mg/L biotin+9.24 mg/L p-aminobenzoic acid+9.24 mg/L folic acid+9.24 mg/L riboflavin+1.93 g/L KH2PO4+0.77 g/L MgSO4-7H2O+15.4 g/L (NH4)2SO4+17.33 g/L sodium butyrate

### Base (for pH Control)

5M NH₄OH

### Overlay

38 mL isopropyl myristate (38% V/VO) was added to tank at 24 hours.
Antifoam Struktol SB2121 (0.1 mL/L) at the beginning of the run

### Fermentation Run Condition:

We used pulse feeding during the run. Fermentation batch was inoculated with 3.5 mL of inoculum (3.5% V/VO). Feeding was triggered at the end of batch phase when batch glucose was completely exhausted and pO2 was increased by 10% (or more). Feed media (production media) was delivered in pulses. Each pulse delivered 1.07 g glucose/starting batch volume with maximum feed rate not exceeding 6.4 g glucose/L/hr. PH was maintained at 5.5 throughout the run. Temperature of fermenter was maintained at 30C. Air flow rate was maintained at 100 mL/min. Agitation was 2000 rpm.

### Summary of Metrics:

### LSC3-426:

Maximum Divarinic acid: 4.85 g/L
Maximum Divarin: 3.0 g/L
Titer/time: 1.54 g Divarin equivalent/L/day
(Divarinic acid) Titer/time: 1.2 g/L/day

### Example 8D

Strain: LSC3-632
Genotype: JK9-3d MATa trp1:: TRP1_S288c leu2::LEU2_S288c gal80Δ::lox72 mig14::lox72 URA3:: (loxHygR)/KIURA3-degtag KIURA3-degtag::pLOA-516 HIS4::pLOA-610

### Fermentation Process Summary:

### Seed Train:

A shake flask containing 50 mL YPD with 20 g/L glucose was inoculated with a seed vial containing LSC3-632. Strains grew at 30 C for 24 hours to an OD600 of 10-12. 3.5 ml of this culture was used to inoculate the fermentation tank.

### Media:

Seed Media: YP with 20 g/L Glucose
Batch media:
10 g/L yeast extract+20 g/L peptones+20 g/L glucose+500 mg/L histidine+12 mg/L myo-inositol+12 mg/L thiamin hydrochloride+12 mg/L pyridoxal hydrochloride+12 mg/L nicotinic acid+12 mg/L calcium pantothenate+0.6 mg/L biotin+12 mg/L p-aminobenzoic acid+0.15 g/L EDTA+7.8 mg/L CuSO4-5H2O+0.0512 g/L FeSO4-7H2O+0.0032 g/L MnCl2+4.77 mg/L Na2MoO4+0.102 g/L ZnSO4-7H2O+0.0086 g/L CoCl2-6H2O+0.0384 g/L CaCl2-2H2O+5.5 g/L KH2PO4+2.9 g/L MgSO4-7H2O+45.1 g/L (NH4)2SO4

### Production Media:

500.5 g/L glucose+337.26 mg/L citric acid monohydrate+1.54 mg/L H3BO3+1 mg/L CuSO4-5H2O+17.25 mg/L FeCl3-6H2O+1 mg/L MnCl2+0.62 mg/L Na2MoO4+8.32 mg/L ZnSO4-7H2O+9.24 mg/L myo-inositol+9.24 mg/L thiamin hydrochloride+9.24 mg/L pyridoxal hydrochloride+9.24 mg/L nicotinic acid+9.24 mg/L calcium pantothenate+9.24 mg/L biotin+9.24 mg/L p-aminobenzoic acid+9.24 mg/L folic acid+9.24 mg/L riboflavin+1.93 g/L KH2PO4+0.77 g/L MgSO4-7H2O+15.4 g/L (NH4)2SO4+17.33 g/L sodium butyrate

### Base (for pH Control)

5M NH₄OH

### Overlay

100 mL autoclaved polypropylene glycol 1200 (PPG1200) (25% V/VO) was added to tank together with 400 mL batch media.

### Fermentation Run Condition:

We used pulse feeding during the run. Fermentation batch was inoculated with 3.5 mL of inoculum (3.5% V/VO). Feeding was triggered at the end of batch phase when batch glucose was completely exhausted and pO2 was increased by 10% (or more). Feed media (production media) was delivered in pulses. Each pulse delivered 1.07 g glucose/starting batch volume with maximum feed rate not exceeding 6.4 g glucose/L/hr. PH was maintained at 5.5 throughout the run. Temperature of fermenter was maintained at 30C. Air flow rate was maintained at 100 mL/min.

### Summary of Metrics:

Maximum divarinic acid: 14 g/L
Maximum divarin: 5.2 g/L
Titer/time: 1.57 g divarinic Acid/L/day

### Example 8E

Strain: LSC3-632

### Fermentation Process Summary:

### Seed Train:

A shake flask containing 50 mL YPD with 20 g/L glucose was inoculated with a seed vial containing LSC3-632. Strains grew at 30 C for 24 hours to an OD600 of 10-12. 3.5 ml of this culture was used to inoculate the fermentation tank.

### Media:

Seed Media: YP with 20 g/L Glucose

### Batch Media:

10 g/L yeast extract+20 g/L peptones+20 g/L glucose+500 mg/L histidine+12 mg/L myo-inositol+12 mg/L thiamin hydrochloride+12 mg/L pyridoxal hydrochloride+12 mg/L nicotinic acid+12 mg/L calcium pantothenate+0.6 mg/L biotin+12 mg/L p-aminobenzoic acid+0.15 g/L EDTA+7.8 mg/L CuSO4-5H2O+0.0512 g/L FeSO4-7H2O+0.0032 g/L MnCl2+4.77 mg/L Na2MoO4+0.102 g/L ZnSO4-7H2O+0.0086 g/L CoCl2-6H2O+0.0384 g/L CaCl2-2H2O+5.5 g/L KH2PO4+2.9 g/L MgSO4-7H2O+45.1 g/L (NH4)2SO4

### Production Media:

500.5 g/L glucose+337.26 mg/L citric acid monohydrate+1.54 mg/L H3BO3+1 mg/L CuSO4-5H2O+17.25 mg/L FeCl3-6H2O+1 mg/L MnCl2+0.62 mg/L Na2MoO4+8.32 mg/L ZnSO4-7H2O+9.24 mg/L myo-inositol+9.24 mg/L thiamin hydrochloride+9.24 mg/L pyridoxal hydrochloride+9.24 mg/L nicotinic acid+9.24 mg/L calcium pantothenate+9.24 mg/L biotin+9.24 mg/L p-aminobenzoic acid+9.24 mg/L folic acid+9.24 mg/L riboflavin+1.93 g/L KH2PO4+0.77 g/L MgSO4-7H2O+15.4 g/L (NH4)2SO4+17.33 g/L sodium butyrate

### Base (for pH Control)

5M NH₄OH

### Overlay

100 ml or 50 ml of autoclaved polypropylene glycol 1200 (PPG1200) (25 or 12.5% V/VO) was added to tanks together with 400 mL batch media.

### Fermentation Run Condition:

We used pulse feeding during the run. Fermentation batch was inoculated with 3.5 ml of inoculum (3.5% V/VO). Feeding was triggered at the end of batch phase when batch glucose was completely exhausted and pO2 was increased by 10% (or more). Feed media (production media) was delivered in pulses. Each pulse delivered 1.07 g glucose/starting batch volume with maximum feed rate not exceeding 6.4 g glucose/L/hr. pH was maintained at 5.5 throughout the run. Temperature of fermenter was maintained at 30C. Air flow rate was maintained at 100 mL/min.

### Summary of Results:

Observed no difference in metrics between 5% and 10% PPG loading.
Maximum divarinic acid (at 120 hours): 7.7-8.2 g/L
Maximum divarin (at 120 hours): 2 g/L
Titer/time: 1.7-1.8 g divarinic acid/L/day

### Example 9: Additional Production Strain Constructions

To allow for additional modifications using the HIS4 marker, strain LSC3-136 was generated by transforming strain LSC3-134 with pLYG-005, which possesses a CEN/ARS origin, G418 resistance marker, and a constitutively expressed Cre recombinase. Plasmid pLYG-005 was cured by identifying spontaneous loss of plasmid via loss of G418 resistance from restruck colonies on YPD agar plates.

To generate strains with higher acyl activating enzyme activity for the butyric acid substrate, a yeast-integrating plasmid, pLOA-419, was generated containing a single gene expression cassette consisting of a pGAL1-10 bidirectional promoter driving AtAAE7 from pGAL10 (with a S. cerevisiae GRE3 terminator; tScGRE3) and no gene from pGAL1 (with tScCYC1), a HIS4 marker from S. cerevisiae, and E. coli pUC origin and ampicillin resistance marker. To construct pLOA-419, two fragments were PCR amplified from plasmid pLOA-327 with primers YO056 and YO11658 (5719 bp), and YO11426 and YO12782 (3124 bp) and assembled in E. coli.

Plasmid pLOA-419 was next linearized by PCR amplification to an 8669 bp fragment using primers YO12913 and YO12914. Alternatively, pLOA-419 was linearized by treatment with ZraI (New England Biolabs) restriction enzyme per the manufacturer's directions, which has a single cut site in the HIS4 marker. Fragments were individually transformed into strain LSC3-136 and plated on YNB agar containing CSM-His dropout supplement. Individual colonies were screened with the Delft medium containing 1× YP instead of CSM and containing 150 mM MES pH 5.5 and the potassium phosphate stock also adjusted to pH 5.5 for divarinic acid production, with sampling of the production cultures after 18 hours. Strain LSC3-426 was isolated as one of the screened colonies with better performance metrics (higher total divarin equivalents).

To generate strains with higher olivetolic/divarinic acid cyclase activity, a YIP, pLOA-425, was generated containing a single gene expression cassette consisting of a pGAL1-10 bidirectional promoter driving a non-functional CsTKS-C₁₅₇S mutant fused to CsOAC2 via a T2A self-cleaving peptide from pGAL1 (with tScCYC1), and no gene from pGAL10 (with tScGRE3), a HIS4 marker from S. cerevisiae, and E. coli pUC origin and ampicillin resistance marker. To construct pLOA-425, two fragments were PCR amplified, one from plasmid pLOA-164 (described below) with primers YO056 and YO11658 (5719 bp), and one from pLOA-327 with primers YO11426 and YO12782 (3124 bp). These fragments were assembled in E. coli.

Plasmid pLOA-425 was next linearized by PCR amplification to an 8408 bp fragment using primers YO12913 and YO12914. Alternatively, pLOA-425 was linearized by treatment with ZraI (New England Biolabs) restriction enzyme per the manufacturer's directions, which has a single cut site in the HIS4 marker. Fragments were transformed into strain LSC3-136 and plated on YNB agar containing CSM-His dropout supplement. Individual colonies were screened with the Delft medium containing 1× YP instead of CSM, with sampling of the production culture after 18 hours. Strain LSC3-433 was isolated as one of the screened colonies with better production metrics (higher olivetolic acid titer).

To integrate full pathway cassettes at high copy number via a single transformation in the S. cerevisiae genome, a high copy number multi-integration system was developed based on first introducing a “landing pad” into the genome for the yeast integrating plasmid (YIP) integration. The landing pad was integrated in place of the non-functional native URA3 genetic marker encoding orotidine-5′-phosphate decarboxylase (URA3) from Kluyveromyces lactis (KIURA3) with a 3′ protein degradation tag (Maury et al., PLOS ONE 11 (3): e0150394, 2016), with a premature stop codon introduced in the KIURA3 gene (KIURA3*-degtag) and an adjacent upstream hygromycin resistance marker (HygR, consisting of hph encoding hygromycin-B 4-O-kinase from Escherichia coli flanked by a TEF1 promoter and TEF1 terminator from Ashbya gossypii) flanked by lox sites (herein referred to as loxHygR). The KIURA3 gene is flanked by a KIURA3 promoter and terminator from K. lactis. Plasmid pLOA-400 was first cloned from three fragments to generate this integration cassette. The KIURA3 gene cassette was amplified as two fragments using primers YO12273 and YO12272, and YO12271 and YO12274, from template G-12 ordered as a gBlock from Integrated DNA technologies (Coralville, IA), with the premature stop codon introduced via primers at the location of the internal split. The backbone vector fragment contained an ampicillin resistance marker, E. coli pUC origin, and loxHygR, and was amplified from the internal plasmid pLOA-094 using primers YO12275 and YO1106. Strain LSC3-522 was generated by introducing the loxHygR/KIURA3-defective cassette as two split linear PCR fragments (1213 bp and 2177 bp) at the non-functional URA3 locus in strain LSC3-297 using template pLOA-400 and primers YO12281 and YO343, and YO189 and YO12282. Sanger sequencing was performed on colony PCR products from resulting transformants to validate successful and error-free insertion of the loxHygR/KIURA3-defective cassette at the URA3 locus. Strain LSC3-434 was generated by transforming the same fragments as described for LSC3-522 into parent strain LSC3-357 (S. cerevisiae JK9-3d MATa with deletions of the GAL80 and MIG1 loci and repaired auxotrophies of LEU2 and TRP1 using alleles amplified from S. cerevisiae S288c, and repaired auxotrophy of HIS4 using the allele amplified from S. cerevisiae CEN.PK2-1C MATa).

An artificial YIP, pLOA-516 (containing a yeast CEN/ARS origin and selection marker but linearized by PCR prior to yeast transformation with these elements excluded), was generated that split a functional KIURA3 marker into 5′ and 3′ pieces flanking a full pathway cassette for production of divarinic acid and divarin containing AtAAE7, CsTKS, and CsOAC2, as described above. The remainder of the plasmid contains a CEN/ARS origin, a TRP1 selection marker including native promoter and terminator from S. cerevisiae S288c, and an ampicillin resistance marker and pUC origin for plasmid maintenance in E. coli. pLOA-516 was assembled by recombinational cloning of 4 PCR fragments in S. cerevisiae CEN.PK2-1C MATa (as described for yeast transformation, with 100 ng of the largest PCR fragment and 1:1 molar normalized amounts of all other fragments). These fragments were 6089 bp amplified from pLOA-445 using primers YO12715 and YO12799, 700 bp amplified from pLOA-164 using primers YO12798 and YO12790, 1223 bp amplified from pLOA-057 using primers YO12156 and YO12190, and 2608 bp amplified from pLOA-327 using primers YO12714 and YO12716.

Several plasmid constructions are here described that were templates used in the construction of pLOA-516. Plasmid pLOA-057 was constructed from three PCR fragments amplified as described previously, assembling a 6875 base pair fragment containing the CEN/ARS HIS4 expression plasmid backbone and CYC1 terminator from pLOA-054 using primers YO11589 and YO12052, a 219 base pair fragment containing the GRE3 terminator from internal plasmid s991 using primers YO12051 and YO11582, and a 1924 base pair fragment containing pGAL1-10 and CsTKS1 using primers YO11581 and YO11444. Plasmid pLOA-164 contained an alternative OAC from C. sativa matching the deposited protein sequence on the UniProt database (CsOAC2). This CsOAC differs from CsOAC1 by the addition of an asparagine inserted at position 50 of the protein sequence (addition of an AAT codon from positions 148-150 in the nucleotide sequence). It was constructed from a 4725 base pair fragment amplified from pLOA-060 using primers YO11393 and YO12161, and a 5010 base pair fragment amplified from pLOA-060 using primers YO12162 and YO11497, with assembly as described previously. The pathway cassette in pLOA-164 was fully sequence validated by Sanger sequencing. Plasmid pLOA-060, containing an OAC from a strain of C. sativa codon-optimized for S. cerevisiae (CsOAC1), CEN/ARS, and a HIS4 marker, was used as a control in activity screens for other mutants and homologous. It was constructed from three PCR fragments amplified as described above, assembling a 6549 base pair fragment from pLOA-054 containing the HIS4 marker and CEN/ARS using primers YO11447 and YO12052, a 219 base pair fragment from internal plasmid s991 containing the S. cerevisiae GRE3 terminator, and a 2473 base pair fragment containing the pathway cassette minus CsAAE1 from pLOA-035 using primers YO11581 and YO11667. The pathway cassette in pLOA-060 was fully sequence validated by Sanger sequencing. Plasmid pLOA-035 (yeast integrating plasmid where the pathway cassette containing CsTKS-C157S was first generated) was constructed from two PCR fragments as described above, with one 5620 base pair fragment amplified from the yeast integrating plasmid pLOA-007 using primers YO11448 and YO11447, and a 3766 base pair fragment amplified from yeast integrating plasmid pLOA-006 using primers YO11446 and YO11449. pLOA-006 is the same plasmid as pAG304Gal110OSOACCSAAE1 and pLOA-007 is the same plasmid as pAG306Gal110OSOACCSAAE1. Plasmid pLOA-445 is the first artificial KIURA3 YIP constructed, containing a full pathway cassette for olivetolic acid and olivetol production with CsAAE1 and CsOAC1 in place of AtAAE7 and CsOAC2, respectively, and it served as a template for amplification of pre-assembled backbone fragments. Plasmid pLOA-445 was assembled by yeast recombinational cloning of 6 fragments: a 4366 bp fragment amplified from pLOA-417 using primers YO12802 and YO12797, a 670 bp fragment amplified from gBlock G-12 using primers YO12796 and YO12799, a 1858 bp fragment amplified from pLOA-049 using primers YO12798 and YO12190, a 3070 bp fragment amplified from pLOA-051 using primers YO12714 and YO12716, a 937 bp fragment amplified from gBlock G-12 using primers YO12715 and YO12801, and a 305 bp fragment amplified from pLOA-094 using primers YO12800 and YO12803. Plasmid pLOA-417 was used as a template for additional assembled backbone parts and was assembled by yeast recombinational cloning of 7 fragments: a 2625 bp fragment from pLOA-073 using primers YO12739 and YO12713, a 1858 bp fragment from pLOA-049 using primers YO12712 and YO12190, a 3070 bp fragment from pLOA-051 using primers YO12714 and YO12716, a 1531 bp fragment from gBlock G-12 using primers YO12715 and YO12711, a 235 bp fragment from gBlock G-10 using primers YO12710 and YO12736, a 1186 bp fragment from S. cerevisiae S288c genomic DNA using primers YO12735 and YO12738, and a 886 bp fragment from pLOA-054 using primers YO12737 and YO12740. The presence of correct cloning junctions was determined by colony PCR, and individual plasmids from yeast were purified using either a Zymoprep™ Yeast Plasmid Miniprep II kit (Zymo Research, Orange, CA) or a Quick-DNA fungal/bacterial miniprep kit (Zymo Research, Orange, CA). 5 uL of purified plasmid product from yeast was transformed into chemically competent E. coli (NEB 5-alpha). Higher concentration purified plasmid was mini-prepped per a standard column protocol and the full pathway cassette and KIURA3 marker sequence was verified by Sanger sequencing. Plasmid pLOA-051 was generated by assembling a 4756 bp fragment from template pLOA-035 using primers YO11576 and YO11577, and a 5797 bp fragment from pLOA-046 using primers YO11578 and YO11575.

To generate strains with both higher tetraketide synthase activity, plasmid pLOA-610 was generated containing a single gene expression cassette consisting of a pGAL1-10 bidirectional promoter driving expression of CsTKS (with tScCYC1), and no gene following pGAL10 (with tScGRE3). Plasmids additionally contained a HIS4 marker from S. cerevisiae, and E. coli pUC origin and ampicillin resistance marker. To construct pLOA-610, two fragments were PCR amplified from plasmid pLOA-057 with primers YO056 and YO11658 (5180 bp), and YO11426 and YO12782 (3124 bp) and assembled and transformed into E. coli as described above. These fragments were assembled and transformed into E. coli as described previously. Plasmid pLOA-460 contained CsAAE1 with the native DNA sequence from C. sativa (not present in the fragments used to construct pLOA-620) under control of pGAL10, with CEN/ARS, HIS4 from S. cerevisiae CEN.PK2-1C, and an E. coli pUC origin and ampicillin resistance marker, and was constructed from a 7741 bp fragment amplified from pLOA-059 with primers YO12279 and YO12297, a 1093 bp fragment amplified from a synthetic DNA fragment G-136 (ordered from Twist Bioscience, South San Francisco, CA) with primers YO12758 and YO12833, and a 1200 bp fragment amplified from a synthetic DNA fragment G-135 (ordered from Twist Bioscience) with primers YO12834 and YO12759. Assembly and transformation into E. coli was performed as previously described.

A 5793 bp integration fragment from the artificial YIP pLOA-516 was linearized by PCR amplification using primers YO12853 and YO12854. This generated split KIURA3 marker homologous regions flanking the expression cassette for AtAAE7, CsTKS, and CsOAC2 that can integrate and tandem duplicate many times in the defective KIURA3 landing pad site. Additionally, plasmid pLOA-610 was linearized by PCR amplification using primers YO12913 and YO12914 to generate an 8130 bp fragment. The two YIP fragments derived from pLOA-516 and pLOA-610 were simultaneously transformed into LSC3-522 and cells were plated on YNB agar plates containing CSM-His-Ura dropout supplement. Individual colonies were screened as described above at pH 5.5 for divarinic acid/divarin production, and strain LSC3-632 was isolated.

Other strains such as LSC3-520 were generated by only transforming the 5793 bp integration fragment from artificial YIP pLOA-516 as described above into strain LSC3-434, with transformants plated on YNB agar plates containing CSM-Ura dropout supplement. LSC3-520 was isolated from screening individual colonies as described above at pH 5.5 for divarinic acid/divarin production.

1. A process comprising:
contacting an aqueous phase comprising glucose and hexanoic acid or a salt thereof and
an organic phase immiscible with the aqueous phase
with a recombinant, heterologous microorganism comprising one or more of a polypeptide having: at least 95% sequence identity with Cannabis sativa olivetol synthase (which is a tetraketide synthase, csOLS), at least 95% sequence identity with Cannabis sativa olivetolic acid cyclase (csOAC), and at least 95% sequence identity with an acyl activating enzyme (AAE) selected from SEQ ID NO:8, SEQ ID NO:9, SEQ ID NO: 10, or SEQ ID NO:11,
to produce olivetol and olivetolic acid or a salt thereof,
wherein the olivetol and olivetolic acid or the salt thereof are produced in a combined amount of at least about 2 g per liter of total liquid broth (comprising both aqueous and immiscible liquid phases) after 1-7 days of operation.

2. The process of claim 1, wherein the fermenting is performed in the absence of galactose.

3. The process of claim 1, wherein the aqueous phase comprises galactose.

4. The process of claim 1, wherein the organic phase comprises an alkane, an alcohol with carbon number greater than 4, an ester (such as isopropyl myristate), a triglyceride (including commercially available vegetable oils such as sunflower oil, soybean oil, or olive oil), a diester, a ketone, or a polyether (such as a polyglyme).

5. The process of claim 1, wherein the aqueous phase further comprises histidine.

6. The process of claim 1, wherein the pH of the aqueous phase is at a pH of about 4 to about 8.

7. The process of claim 1, wherein the microorganism is Saccharomyces cerevisiae.

8. The process of claim 1, wherein the fermentation is performed in a semi-continuous mode (“fill-and-draw”), or a continuous mode, for a prolonged duration, and the overall combined productivity of olivetol and olivetolate is >0.3 g per L of total volume (including aqueous and immiscible liquid phases) per day of operation.

9. A process comprising:
contacting an aqueous phase comprising glucose and butyric acid (CH₃(CH₂)₂CO₂H) or a salt thereof and
an organic phase immiscible with the aqueous phase
with a recombinant, heterologous microorganism comprising a polypeptide having: at least 95% sequence identity with a one or more of a Cannabis sativa olivetol synthase (which is a tetraketide synthase, csOLS), at least 95% sequence identity with a Cannabis sativa olivetolic acid cyclase (csOAC), and at least 95% sequence identity with an acyl activating enzyme (AAE) selected from SEQ ID NO:8, SEQ ID NO:9, SEQ ID NO:10, or SEQ ID NO: 11,
to produce divarin and/or divarinic acid or a salt thereof.

10. The process of claim 9, wherein the fermenting is performed in the absence of galactose.

11. The process of claim 9, wherein the aqueous phase comprises galactose.

12. The process of claim 9, wherein the organic phase comprises an alkane, an alcohol with carbon number greater than 4, an ester (such as isopropyl myristate), a triglyceride (including commercially available vegetable oils such as sunflower oil, soybean oil, or olive oil), a diester, or a ketone.

13. The process of claim 9, wherein the aqueous phase further comprises histidine.

14. The process of claim 9, wherein the pH of the aqueous phase is at a pH of about 4 to about 8.

15. The process of claim 9, wherein the microorganism is Saccharomyces cerevisiae.

16. The process of claim 9, wherein the fermentation is performed in a semi-continuous mode (“fill-and draw”), or a continuous mode, for a prolonged duration.

17. A process comprising:
contacting an aqueous phase comprising glucose and a carboxylic acid of formula RCO₂H or a salt thereof, wherein R is optionally substituted C₁-C₈ alkyl, optionally substituted C₂-C₆ alkenyl, or optionally substituted C₂-C₈ alkynyl and
an organic phase immiscible with the aqueous phase
with a recombinant, heterologous microorganism comprising one or more of a polypeptide having: at least 95% sequence identity with a Cannabis sativa olivetol synthase (which is a tetraketide synthase, csOLS), at least 95% sequence identity with a Cannabis sativa olivetolic acid cyclase (csOAC), and at least 95% sequence identity with an acyl activating enzyme (csAAE) selected from SEQ ID NO:8, SEQ ID NO:9, SEQ ID NO:10, or SEQ ID NO: 11,
to produce a compound of formula (IA) and/or (IB): 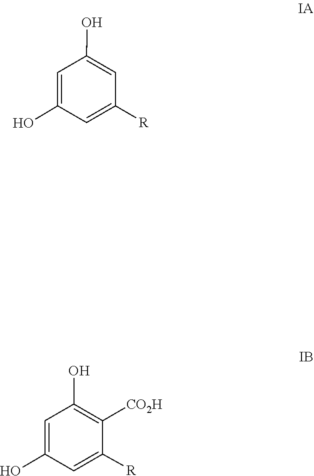
or a salt thereof wherein R is defined as above.

18. The process of claim 17, wherein the fermenting is performed in the absence of galactose.

19. The process of claim 17, wherein the aqueous phase comprises galactose.

20. The process of claim 17, wherein the organic phase comprises an alkane, an alcohol with carbon number greater than 4, an ester (such as isopropyl myristate), a triglyceride (including commercially available vegetable oils such as sunflower oil, soybean oil, or olive oil), a diester, a ketone, or a polyether (such as a polyglyme).

21. The process of any one of claim 1, 9, or 17, wherein the organic phase immiscible with the aqueous phase comprises polypropylene glycol (PPG).

22. The process of claim 21, wherein the PPG has an average molecular weight of greater that about 1,200.

23. The process of claim 21, wherein the PPG has an average molecular weight of about 1,200, about 1,500, or about 4,000.

24. A process comprising fermenting a recombinant, heterologous fungus, to produce a compound of formula (IA) and/or (IB): 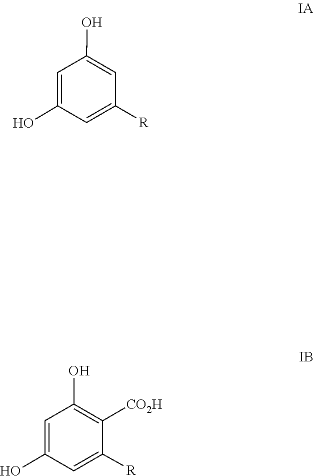
or a salt thereof wherein R is optionally substituted C₁-C₈ alkyl or optionally substituted C₂-C₆ alkenyl.

25. The process of claim 1, wherein HRPKS, NRPKS and (pseudoacyl carrier protein)-thioesterase enzymes are co-expressed in the fungus.

26. The process of claim 1, wherein the fungus is Aspergillus nidulans.
